SEQUENCE LISTING (64 sequences)

SEQ ID NO: 1 - protein, 475 aa, Homo sapiens
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 2 - protein, 448 aa, Homo sapiens
ADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 3 - protein, 446 aa, Homo sapiens
ADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKK

SEQ ID NO: 4 - protein, 298 aa, Homo sapiens
IESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRS

SEQ ID NO: 5 - protein, 184 aa, Homo sapiens
MKALGAVLLALLLCGRPGRGQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGKGAGGPRGSSETVGAALLLNLLAGLGAMGARRP

SEQ ID NO: 6 - protein, 164 aa, Homo sapiens
QTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGKGAGGPRGSSETVGAALLLNLLAGLGAMGARRP

SEQ ID NO: 7 - protein, 131 aa, Homo sapiens
QTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTG

SEQ ID NO: 8 - protein, 151 aa, Homo sapiens
MKALGAVLLALLLCGRPGRGQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTG

SEQ ID NO: 9 - protein, 86 aa, Homo sapiens
LRCYTCKSLPRDERCNLTQNCSHGQTCTTLIAHGNTESGLLTTHSTWCTDSCQPITKTVEGTQVTMTCCQSSLCNVPPWQSSRVQD

SEQ ID NO: 10 - protein, 140 aa, Homo sapiens
QTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGKGAGGPRGS

SEQ ID NO: 11 - protein, 5 aa, Artificial Sequence
  source: /note=“Description of Artificial Sequence Synthetic peptide”
GSGGG

SEQ ID NO: 12 - protein, 5 aa, Artificial Sequence
  source: /note=“Description of Artificial Sequence Synthetic peptide”
GSGGG

SEQ ID NO: 13 - protein, 4 aa, Artificial Sequence
  source: /note=“Description of Artificial Sequence Synthetic peptide”
GSGG

SEQ ID NO: 14 - protein, 4 aa, Artificial Sequence
  source: /note=“Description of Artificial Sequence Synthetic peptide”
SGGG

SEQ ID NO: 15 - protein, 100 aa, Artificial Sequence
  source: /note=“Description of Artificial Sequence Synthetic polypeptide”
  SITE (1)..(100): /note=“This sequence may encompass 1-20 ”Gly Gly Gly Gly Ser“ repeating units”
  source: /note=“See specification as filed for detailed description of substitutions and preferred embodiments”
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 16 - protein, 20 aa, Artificial Sequence
  source: /note=“Description of Artificial Sequence Synthetic peptide”
GGGGSGGGGSGGGGSGGGGS

SEQ ID NO: 17 - protein, 15 aa, Artificial Sequence
  source: /note=“Description of Artificial Sequence Synthetic peptide”
GGGGSGGGGSGGGGS

SEQ ID NO: 18 - protein, 5 aa, Artificial Sequence
  source: /note=“Description of Artificial Sequence Synthetic peptide”
GPPGS

SEQ ID NO: 19 - protein, 4 aa, Artificial Sequence
  source: /note=“Description of Artificial Sequence Synthetic peptide”
GGGS

SEQ ID NO: 20 - protein, 3 aa, Artificial Sequence
  source: /note=“Description of Artificial Sequence Synthetic peptide”
GYS

SEQ ID NO: 21 - protein, 2 aa, Artificial Sequence
  source: /note=“Description of Artificial Sequence Synthetic peptide”
GS

SEQ ID NO: 22 - protein, 5 aa, Artificial Sequence
  source: /note=“Description of Artificial Sequence Synthetic peptide”
SGGGG

SEQ ID NO: 23 - protein, 3 aa, Artificial Sequence
  source: /note=“Description of Artificial Sequence Synthetic peptide”
SGG

SEQ ID NO: 24 - protein, 2 aa, Artificial Sequence
  source: /note=“Description of Artificial Sequence Synthetic peptide”
SG

SEQ ID NO: 25 - protein, 5 aa, Artificial Sequence
  source: /note=“Description of Artificial Sequence Synthetic peptide”
GGGGA

SEQ ID NO: 26 - protein, 4 aa, Artificial Sequence
  source: /note=“Description of Artificial Sequence Synthetic peptide”
GGGA

SEQ ID NO: 27 - protein, 5 aa, Artificial Sequence
  source: /note=“Description of Artificial Sequence Synthetic peptide”
EAAAK

SEQ ID NO: 28 - protein, 6 aa, Artificial Sequence
  source: /note=“Description of Artificial Sequence Synthetic 6xHis tag”
HHHHHH

SEQ ID NO: 29 - protein, 8 aa, Artificial Sequence
  source: /note=“Description of Artificial Sequence Synthetic peptide”
DYKDDDDK

SEQ ID NO: 30 - protein, 15 aa, Artificial Sequence
  source: /note=“Description of Artificial Sequence Synthetic peptide”
GLNDIFEAQKIEWHE

SEQ ID NO: 31 - protein, 31 aa, Artificial Sequence
  source: /note=“Description of Artificial Sequence Synthetic polypeptide”
GLNDIFEAQKIEWHEGGHHHHHHDYKDDDDK

SEQ ID NO: 32 - protein, 31 aa, Artificial Sequence
  source: /note=“Description of Artificial Sequence Synthetic polypeptide”
DYKDDDDKHHHHHHGGGLNDIFEAQKIEWHE

SEQ ID NO: 33 - protein, 650 aa, Artificial Sequence
  source: /note=“Description of Artificial Sequence Synthetic polypeptide”
METDTLLLWVLLLWVPGSTGGLNDIFEAQKIEWHEGGHHHHHHDYKDDDDKADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTG

SEQ ID NO: 34 - protein, 630 aa, Artificial Sequence
  source: /note=“Description of Artificial Sequence Synthetic polypeptide”
GLNDIFEAQKIEWHEGGHHHHHHDYKDDDDKADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTG

SEQ ID NO: 35 - protein, 880 aa, Artificial Sequence
  source: /note=“Description of Artificial Sequence Synthetic polypeptide”
METDTLLLWVLLLWVPGSTGGLNDIFEAQKIEWHEGGHHHHHHDYKDDDDKADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKASSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGGGGGSGGGGSGGGGSSSAAASSSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAAHHHHHH

SEQ ID NO: 36 - protein, 1280 aa, Artificial Sequence
  source: /note=“Description of Artificial Sequence Synthetic polypeptide”
METDTLLLWVLLLWVPGSTGGLNDIFEAQKIEWHEGGHHHHHHDYKDDDDKADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKASSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGGGGGSGGGGSGGGGSSSAAASSSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAAHHHHHH

SEQ ID NO: 37 - protein, 916 aa, Artificial Sequence
  source: /note=“Description of Artificial Sequence Synthetic polypeptide”
METDTLLLWVLLLWVPGSTGGLNDIFEAQKIEWHEGGHHHHHHDYKDDDDKADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKASSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKGGGGSGGGGSGGGGSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 38 - protein, 879 aa, Artificial Sequence
  source: /note=“Description of Artificial Sequence Synthetic polypeptide”
METDTLLLWVLLLWVPGSTGEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGDYKDDDDKHHHHHHGGGLNDIFEAQKIEWHE

SEQ ID NO: 39 - protein, 884 aa, Artificial Sequence
  source: /note=“Description of Artificial Sequence Synthetic polypeptide”
METDTLLLWVLLLWVPGSTGEVQLLESGGGLVQPGGSLRLSCAASGFTFSDYAMSWVRQAPGKGLEWVSAISYSGSYTYYADSVKGRFTISRDNSKNTLYLQMNSLRAEDTAVYYCARGRYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGDYKDDDDKHHHHHHGGGLNDIFEAQKIEWHE

SEQ ID NO: 40 - protein, 599 aa, Artificial Sequence
  source: /note=“Description of Artificial Sequence Synthetic polypeptide”
ADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTG

SEQ ID NO: 41 - protein, 454 aa, Artificial Sequence
  source: /note=“Description of Artificial Sequence Synthetic polypeptide”
HHHHHHADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 42 - protein, 479 aa, Artificial Sequence
  source: /note=“Description of Artificial Sequence Synthetic polypeptide”
ADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGDYKDDDDKHHHHHHGGGLNDIFEAQKIEWHE

SEQ ID NO: 43 - protein, 412 aa, Artificial Sequence
  source: /note=“Description of Artificial Sequence Synthetic polypeptide”
GPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAASDYKDDDDKHHHHHHGGGLNDIFEAQKIEWHE

SEQ ID NO: 44 - DNA, 1953 bp, Artificial Sequence
  source: /note=“Description of Artificial Sequence Synthetic polynucleotide”
atggaaaccgacaccctgctgctgtgggtgctgctgctgtgggtgccaggatctacaggcgccgaccagcggagagacttcatcgacatcgagagcaagttcgccctgcggacccctgaggataccgccgaggatacctgccacctgattccaggcgtggccgagagcgtggccacctgtcacttcaaccacagctccaagaccttcatggtcatccacggctggaccgtgaccgggatgtacgagagctgggtgccaaaactggtggccgccctgtacaagcgcgagcccgacagcaatgtgatcgtggtggactggctgagcagagcccaggaacactaccctgtgtccgccggctacaccaagctcgtgggacaggacgtggcccggttcatcaactggatggaagaagagttcaactaccccctggacaatgtgcatctgctgggctacagcctgggagcccatgccgctggaattgccggcagcctgaccaacaagaaagtgaaccggatcaccggcctggaccctgccggccctaatttcgagtatgccgaggcccccagcagactgagccccgacgatgccgatttcgtggacgtgctgcacaccttcaccagaggaagccccggcagatccatcggcatccagaaacctgtgggccacgtggacatctaccccaacggcggcacatttcagcccggctgcaatatcggcgaggccatcagagtgatcgccgagaggggcctgggagatgtggatcagctcgtgaagtgcagccacgagcggagcatccacctgttcatcgactccctgctgaacgaggaaaaccccagcaaggcctaccggtgcagcagcaaagaggccttcgagaagggcctgtgcctgagctgccggaagaaccggtgcaacaacctgggctacgagatcaacaaagtgcgggccaagcggagcagcaagatgtacctgaaaacccggtcccagatgccctacaaggtgttccattatcaagtgaagatccacttcagcggcaccgagagcgagacacacaccaaccaggcctttgagatcagcctgtacggcaccgtggccgaatccgagaacatccccttcaccctgcccgaggtgtccacaaacaagacctacagcttcctgatctacaccgaggtggacatcggcgagctgctgatgctgaagctgaaatggaagtccgacagctacttctcttggagcgactggtggtccagccccggcttcgccattcagaaaattagagtgaaggccggcgaaacccagaaaaaagtgatcttctgctcccgcgagaaggtgtcccatctgcagaagggcaaagcccctgccgtgtttgtgaagtgccacgacaagagcctgaacaagaagtctggcggcggaggcggatctgggggaggcggaagtggcgggggaggatcagggggcggaggatctcagacccagcaggaagaggaagaagaggacgaggaccacggccccgatgactacgacgaagaggatgaggatgaggtggaagaagaagagacaaaccggctgcctggcggcaggtccagagtgctgctgagatgctacacatgcaagtccctgccccgggacgagcggtgcaacctgacacagaattgctcccacggccagacctgcaccaccctgatcgcccacggcaataccgagtctggcctgctgaccacccactccacctggtgcaccgatagctgccagcccatcaccaagaccgtggaaggcacccaagtgaccatgacctgctgccagtccagcctgtgcaacgtgccaccttggcagagcagcagagtgcaggaccccaccggcgactacaaggacgacgacgacaagcaccaccaccatcaccacggcggaggactgaacgacatcttcgaggcccagaaaatcgagtggcacgaatga

SEQ ID NO: 45 - protein, 448 aa, Artificial Sequence
  source: /note=“Description of Artificial Sequence Synthetic polypeptide”
ADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVAAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 46 - protein, 650 aa, Artificial Sequence
  source: /note=“Description of Artificial Sequence Synthetic polypeptide”
METDTLLLWVLLLWVPGSTGGLNDIFEAQKIEWHEGGHHHHHHDYKDDDDKADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVAAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTG

SEQ ID NO: 47 - protein, 630 aa, Artificial Sequence
  source: /note=“Description of Artificial Sequence Synthetic polypeptide”
GLNDIFEAQKIEWHEGGHHHHHHDYKDDDDKADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVAAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTG

SEQ ID NO: 48 - protein, 599 aa, Artificial Sequence
  source: /note=“Description of Artificial Sequence Synthetic polypeptide”
ADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVAAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTG

SEQ ID NO: 49 - protein, 162 aa, Artificial Sequence
  source: /note=“Description of Artificial Sequence Synthetic polypeptide”
QTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGDYKDDDDKHHHHHHGGGLNDIFEAQKIEWHE

SEQ ID NO: 50 - protein, 475 aa, Artificial Sequence
  source: /note=“Description of Artificial Sequence Synthetic polypeptide”
SRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFEDYKDDDDKHHHHHHGGGLNDIFEAQKIEWHE

SEQ ID NO: 51 - protein, 650 aa, Artificial Sequence
  source: /note=“Description of Artificial Sequence Synthetic polypeptide”
METDTLLLWVLLLWVPGSTGADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGDYKDDDDKHHHHHHGGGLNDIFEAQKIEWHE

SEQ ID NO: 52 - protein, 20 aa, Artificial Sequence
  source: /note=“Description of Artificial Sequence Synthetic peptide”
METDTLLLWVLLLWVPGSTG

SEQ ID NO: 53 - protein, 630 aa, Artificial Sequence
  source: /note=“Description of Artificial Sequence Synthetic polypeptide”
ADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSGGGGGSGGGGSGGGGSGGGGSQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGDYKDDDDKHHHHHHGGGLNDIFEAQKIEWHE

SEQ ID NO: 54 - protein, 599 aa, Artificial Sequence; identical to SEQ ID NO: 40
  source: /note=“Description of Artificial Sequence Synthetic polypeptide”

SEQ ID NO: 55 - protein, 599 aa, Artificial Sequence; identical to SEQ ID NO: 40
  source: /note=“Description of Artificial Sequence Synthetic polypeptide”

SEQ ID NO: 56 - protein, 4 aa, Homo sapiens
RAKR

SEQ ID NO: 57 - protein, 4 aa, Artificial Sequence
  source: /note=“Description of Artificial Sequence Synthetic peptide”
GGGG

SEQ ID NO: 58 - protein, 5 aa, Artificial Sequence
  source: /note=“Description of Artificial Sequence Synthetic peptide”
GGGGG

SEQ ID NO: 59 - protein, 100 aa, Artificial Sequence
  source: /note=“Description of Artificial Sequence Synthetic polypeptide”
  SITE (1)..(100): /note=“This sequence may encompass 1-20 ”Gly Pro Pro Gly Ser“ repeating units”
  source: /note=“See specification as filed for detailed description of substitutions and preferred embodiments”
GPPGSGPPGSGPPGSGPPGSGPPGSGPPGSGPPGSGPPGSGPPGSGPPGSGPPGSGPPGSGPPGSGPPGSGPPGSGPPGSGPPGSGPPGSGPPGSGPPGS

SEQ ID NO: 60 - protein, 11 aa, Artificial Sequence
  source: /note=“Description of Artificial Sequence Synthetic peptide”
GGGSGGGGSGS

SEQ ID NO: 61 - protein, 10 aa, Artificial Sequence
  source: /note=“Description of Artificial Sequence Synthetic peptide”
  SITE (1)..(10): /note=“This sequence may encompass 5-10 residues”
HHHHHHHHHH

SEQ ID NO: 62 - protein, 33 aa, Homo sapiens
GYSLGAHAAGIAGSLTNKKVNRITGLDPAGPNF

SEQ ID NO: 63 - protein, 3 aa, Artificial Sequence
  source: /note=“Description of Artificial Sequence Synthetic peptide”
GSG

SEQ ID NO: 64 - protein, 5 aa, Artificial Sequence
  source: /note=“Description of Artificial Sequence Synthetic peptide”
GGGGS

(19) United States
(12) Patent Application Publication
(10) Pub. No.: US 2022/0025344 A1
(43) Pub. Date: Jan. 27, 2022

(54) LPL-GPIHBP1 FUSION POLYPEPTIDES

(71) Applicant: NOVARTIS AG, Basel (CH)

(72) Inventors: Jiaping GAO, Ashland, MA (US); Amitabh NIMONKAR, Kingston, MA (US); John TRAUGER, Cambridge, MA (US); Andrei Igorevich VOZNESENSKY, Arlington, MA (US); Stephen Craig WELDON, Leominster, MA (US)

(21) Appl. No.: 17/296,369

(22) PCT Filed: Nov. 25, 2019
(86) PCT No.: PCT/IB2019/060140

§ 371 Date: May 24, 2021

Related U.S. Application Data
(60) Provisional application No. 62/771,359, filed on Nov. 26, 2018.

(51) Int. Cl. C12N 9/20; C07K 14/705; A61P 3/06

(52) U.S. Cl. CPC C12N 9/20; C12Y 301/01034; C07K 14/705; A61K 38/00; C07K 2319/35; C07K 2319/21; C07K 2319/43; A61P 3/06

(57) ABSTRACT

This disclosure relates to fusion polypeptides comprising lipoprotein lipase (LPL) and glycosylphosphatidylinositol-anchored High Density Lipoprotein-binding protein 1 (GPIHBP1). The disclosure also relates to uses of such fusion polypeptides in treating disease such as familial chylomicronemia syndrome (FCS).

### TECHNICAL FIELD

This disclosure relates to fusion polypeptides comprising lipoprotein lipase (LPL) and glycosylphosphatidylinositol-anchored High Density Lipoprotein-binding protein 1 (GPIHBP1). The disclosure also relates to uses of such fusion polypeptides in treating disease such as familial chylomicronemia syndrome (FCS).

### BACKGROUND

Familial chylomicronemia syndrome (FCS) is a rare genetic disorder caused by lipoprotein lipase (LPL) deficiency and characterized by abnormally high level of plasma triglycerides (TG). FCS patients present with childhood-onset severe hypertriglyceridemia (>1,000 mg/dL), episodes of abdominal pain, recurrent acute pancreatitis (AP), eruptive cutaneous xanthomata, lipemia retinalis, and hepatosplenomegaly. AP is a frequent and severe manifestation of FCS (Davidson et al., 2018, J. Clin. Lipidol, 12(4):898-907.e2). The risk of AP progressively increases as TG levels increase (Nawaz et al., 2015, Am J Gastroenterol 110:1497-1503). Mortality rate in hyperlipidemic pancreatitis (HTAP) can be as high as 20-30% (Gubensek et al., 2014, PLoS One 9, e102748).

Lipoprotein lipase (LPL) is a member of the lipase gene family. LPL is a triglyceride lipase secreted primarily by adipocytes, skeletal muscle cells, and cardiomyocytes. LPL folding is mediated by the chaperone lipase maturation factor 1 (LMF1). LPL is secreted into the subendothelial space and then translocated to the lumen of capillaries by glycosylphosphatidylinositol HDL-binding protein 1 (GPIHBP1). After translocation, LPL is tethered to the endothelial cells by heparan sulfated proteoglycans or GPIHBP1. Tethered LPL catalyzes hydrolysis of triglycerides (TG) carried in very low density lipoproteins (VLDL) and chylomicrons (CM) (Savonen et al., 2015, J Lipid Res 56:588-598; Goulbourne et al., 2014, Cell Metab 18:389-396). Free fatty acids liberated by LPL are used as a source of energy by heart and muscle tissue or are stored in the form of TG by adipose tissue. LPL is a tightly controlled enzyme that is stimulated by agonists (ApoC2) and inhibited by antagonists (ANGPTL3, ANGPTL 4, ANGPTL 8) (He et al., 2018, Clin Chim Acta 480:126-137). Loss-of-function mutations in LPL, GPIHBP1, LMF1 lead to LPL deficiency which causes accumulation of TG-rich CM in the blood.

No specific approved pharmacological intervention has been demonstrated to improve the clinical course of hyperlipidemic pancreatitis (HTAP). Therapeutic options for acutely lowering TG to <1000 mg/dL for the treatment of HTAP are limited to switching patients to parenteral hypocaloric nutrition combined with supportive care. Plasmapheresis can be used if the equipment is available (Chaudhry et al., 2018, Expert Rev Clin Pharmacol 11:589-598; Gaudet et al., 2013, J Med Econ 16:657-666; Valaiyapathi and Ashraf, 2017, Curr. Pediatr. Rev. 13(4):225-231), but this is very costly. Prevention of HTAP is also difficult to achieve. FCS patients have few options for maintaining plasma TG below 1000 mg/dL to stave off attacks of abdominal pain and HTAP. Such patients must restrict their dietary fat to less than 20 g/day or 15% of total energy intake for their entire lives. Eighty percent (80%) of FCS patients rate this adherence as “very difficult” (Stroes et al., 2017, Atheroscler Suppl 23:1-7).

### SUMMARY OF THE DISCLOSURE

We have surprisingly found that when expressed as a fusion polypeptide with glycosylphosphatidylinositol-anchored High Density Lipoprotein-binding protein 1 (GPIHBP1), lipoprotein lipase (LPL) maintains a high specific activity, does not aggregate, is stable in PBS and is resistant to activation by ANGPTL4.

Thus, in one aspect, the disclosure provides fusion polypeptides comprising (i) a lipoprotein lipase (LPL) polypeptide, or a functional variant thereof and (ii) a glycosylphosphatidylinositol-anchored High Density Lipoprotein-binding protein 1 (GPIHBP1) polypeptide, or a functional variant thereof. The GPIHBP1 polypeptide (or functional variant thereof) may be located C-terminal or N-terminal to the LPL polypeptide (or functional variant thereof). The LPL polypeptide (or functional variant thereof) and GPIHBP1 polypeptide (or functional variant thereof) may be directly fused together, or may be joined by a linker. The fusion polypeptide may further comprise N-terminal or C-terminal amino acid sequences, for example, to assist with purification, to improve expression or to increase half-life. Such N-terminal or C-terminal amino acid sequences might be included in the expression of the polypeptide but removed later, e.g., before administration.

In one embodiment, the disclosure provides a fusion polypeptides having one of the formula (I) or (II), from N-terminus to C-terminus:

A-B₍ₙ₎-C-D₍ₘ₎-E (I), or

A-D₍ₘ₎-C-B₍ₙ₎-E (II), wherein

A=an optional N-terminal sequence

B=LPL polypeptide or functional variant thereof

C=an optional linker sequence

D=GPIHBP1 polypeptide or functional variant thereof

E=an optional C-terminal sequence, wherein

n=an integer from 1 to 3, and

m=an integer from 1 to 3.

In some embodiments, n=1. In some embodiments, m=1. In some embodiments, n=1 and m=1. In certain embodiments, the LPL and/or GPIHBP1 polypeptides are based on mammalian sequences or derivatives thereof. In specific embodiments, the LPL and/or GPIHBP1 polypeptides are based on human sequences or derivatives thereof.

In some embodiments, the functional variant of the LPL polypeptide comprises an amino acid sequence having at least about 80%, at least about 85%, at least about 90%, at least about 95%, at least about 97%, at least about 98%, or at least about 99% sequence identity to the LPL polypeptide of SEQ ID NO: 1 or SEQ ID NO:2. In some embodiments, the functional variant of the LPL polypeptide consists of an amino acid sequence having at least about 80%, at least about 85%, at least about 90%, at least about 95%, at least about 97%, at least about 98%, or at least about 99% sequence identity to the LPL polypeptide of SEQ ID NO: 1 or SEQ ID NO:2. In some embodiments, the functional variant of the LPL polypeptide comprises the amino acid sequence of (i) SEQ ID NO: 2 having one or more (e.g., 1, 2, 3, 4, 5, 6, 7, 8, 9, 10 or more) point mutations that add to, delete, or substitute any of the amino acids of SEQ ID NO: 2, or (ii) SEQ ID NO: 1 having one or more (e.g., 1, 2, 3, 4, 5, 6, 7, 8, 9, 10 or more) point mutations that add to, delete, or substitute any of amino acids 157-189 of SEQ ID NO: 1. In some embodiments, the functional variant of the LPL polypeptide consists of the amino acid sequence of (i) SEQ ID NO: 2 having one or more (e.g., 1, 2, 3, 4, 5, 6, 7, 8, 9, 10 or more) point mutations that add to, delete, or substitute any of the amino acids of SEQ ID NO: 2, or (ii) SEQ ID NO: 1 having one or more (e.g., 1, 2, 3, 4, 5, 6, 7, 8, 9, 10 or more) point mutations that add to, delete, or substitute any of amino acids 157-189 of SEQ ID NO: 1.

In certain embodiments, the functional variant of the LPL polypeptide is a truncated version of SEQ ID NO: 1. In certain embodiments, the functional variant of the LPL polypeptide is a truncated version of SEQ ID NO: 2. In some embodiments, the functional variant of the LPL polypeptide is a truncated version of (i) an amino acid sequence having at least about 80%, at least about 85%, at least about 90%, at least about 95%, at least about 97%, at least about 98%, or at least about 99% sequence identity to SEQ ID NO: 1 or SEQ ID NO:2; (ii) SEQ ID NO: 2 having one or more (e.g., 1, 2, 3, 4, 5, 6, 7, 8, 9, 10 or more) point mutations that add to, delete, or substitute any one of the amino acids of SEQ ID NO: 2; (iii) SEQ ID NO: 1 having one or more (e.g., 1, 2, 3, 4, 5, 6, 7, 8, 9, 10 or more) point mutations that add to, delete, or substitute any one of amino acids 157-189 of SEQ ID NO: 1; (iv) SEQ ID NO: 1; or (v) SEQ ID NO: 2. In some embodiments, the functional variant of the LPL polypeptide is a truncated version of (i) an amino acid sequence having at least about 80%, at least about 85%, at least about 90%, at least about 95%, at least about 97%, at least about 98%, or at least about 99% sequence identity to SEQ ID NO: 1 or SEQ ID NO:2; (ii) SEQ ID NO: 2 having one or more (e.g., 1, 2, 3, 4, 5, 6, 7, 8, 9, 10 or more) point mutations that add to, delete, or substitute any one of the amino acids of SEQ ID NO: 2; (iii) SEQ ID NO: 1 having one or more (e.g., 1, 2, 3, 4, 5, 6, 7, 8, 9, 10 or more) point mutations that add to, delete, or substitute any one of amino acids 157-189 of SEQ ID NO: 1; (iv) SEQ ID NO: 1; or (v) SEQ ID NO: 2, wherein the truncated version of the LPL polypeptide corresponds to the amino acid sequence of a polypeptide comprising or consisting of amino acids 36-335, 35-340, 34-345, 33-350, 32-355, 31-360, 30-365, 29-370, 28-375, 28-380, 28-385, 28-390, 28-395, 28-400, 28-405, 28-410, 28-415, 28-420, 28-425, 28-430, 28-435, 28-440, 28-445, 28-450, 28-455, 28-460, 28-465, or 28-470 of SEQ ID NO: 1.

In some embodiments, the truncated version of the LPL polypeptide corresponds to the amino acid sequence of a polypeptide comprising or consisting of amino acids 36-335, 35-340, 34-345, 33-350, 32-355, 31-360, 30-365, 29-370, 28-375, 28-380, 28-385, 28-390, 28-395, 28-400, 28-405, 28-410, 28-415, 28-420, 28-425, 28-430, 28-435, 28-440, 28-445, 28-450, 28-455, 28-460, 28-465, or 28-470 of SEQ ID NO: 1. In certain embodiments thereof, the truncated version of SEQ ID NO: 1 has at least about 80%, at least about 85%, at least about 90%, at least about 95%, at least about 97%, at least about 98%, or at least about 99% sequence identity to the corresponding region of SEQ ID NO: 1; optionally wherein the truncated version of SEQ ID NO: 1 has one or more (e.g., 1, 2, 3, 4, 5, 6, 7, 8, 9, 10 or more) point mutations that add to, delete, or substitute any one of amino acids 157-189 of SEQ ID NO: 1.

In some embodiments, the LPL polypeptide comprises or consists of any one of SEQ ID NOs: 1, 2, 3, 4, or 45.

In some embodiments, the functional variant of the GPIHBP1 polypeptide comprises an amino acid sequence having at least about 80%, at least about 85%, at least about 90%, at least about 95%, at least about 97%, at least about 98%, or at least about 99% sequence identity to the GPIHBP1 polypeptide of SEQ ID NO: 5, SEQ ID NO: 6, or SEQ ID NO: 7. In some embodiments, the functional variant of the GPIHBP1 polypeptide consists of an amino acid sequence having at least about 80%, at least about 85%, at least about 90%, at least about 95%, at least about 97%, at least about 98%, or at least about 99% sequence identity to the GPIHBP1 polypeptide of SEQ ID NO: 5, SEQ ID NO: 6, or SEQ ID NO: 7. In certain embodiments, the functional variant of the GPIHBP1 polypeptide comprises the amino acid sequence of SEQ ID NO: 7 having one or more (e.g., 1, 2, 3, 4, 5, 6, 7, 8, 9, 10 or more) point mutations that add to, delete, or substitute any one of the amino acids of SEQ ID NO: 7. In certain embodiments, the functional variant of the GPIHBP1 polypeptide consists of the amino acid sequence of SEQ ID NO: 7 having one or more (e.g., 1, 2, 3, 4, 5, 6, 7, 8, 9, 10 or more) point mutations that add to, delete, or substitute any one of the amino acids of SEQ ID NO: 7.

In some embodiments, the functional variant of the GPIHBP1 polypeptide is a truncated version of (i) an amino acid sequence having at least about 80%, at least about 85%, at least about 90%, at least about 95%, at least about 97%, at least about 98%, or at least about 99% sequence identity to the GPIHBP1 polypeptide of SEQ ID NO: 5; or (ii) SEQ ID NO: 5. In some embodiments, the functional variant of the GPIHBP1 polypeptide is a truncated version of (i) an amino acid sequence having at least about 80%, at least about 85%, at least about 90%, at least about 95%, at least about 97%, at least about 98%, or at least about 99% sequence identity to the GPIHBP1 polypeptide of SEQ ID NO: 5; or (ii) SEQ ID NO: 5, wherein the truncated version of the GPIHBP1 polypeptide corresponds to the amino acid sequence of a polypeptide comprising or consisting of amino acids 62-149, 61-150, 60-151, 55-152, 50-153, 45-154, 40-155, 35-156, 30-157, 25-158, or 20-159 of SEQ ID NO: 5.

In certain embodiments, the truncated version of the GPIHBP1 polypeptide corresponds to the amino acid sequence of a polypeptide comprising or consisting of amino acids 62-149, 61-150, 60-151, 55-152, 50-153, 45-154, 40-155, 35-156, 30-157, 25-158, or 20-159 of SEQ ID NO: 5. In certain embodiments, the truncated version of SEQ ID NO: 5 has at least about 80%, at least about 85%, at least about 90%, at least about 95%, at least about 97%, at least about 98%, or at least about 99% sequence identity to the corresponding region of SEQ ID NO: 5.

In some embodiments, the GPIHBP1 polypeptide comprises or consists of any one of SEQ ID NOs: 5, 6, 7, 8, 9, or 10.

In certain embodiments, the LPL polypeptide and GPIHBP1 polypeptide are joined by a linker. In certain embodiments, the LPL polypeptide and GPIHBP1 polypeptide are joined by a linker C. In some embodiments, the linker comprises or consists of the amino acid sequence recited in any one of SEQ ID NOs: 11-27. In some embodiments, the linker comprises or consists of the amino acid sequence recited in SEQ ID NO: 16 or SEQ ID NO: 17. In some embodiments, the linker comprises or consists of one or more of the amino acid sequences recited in any one of SEQ ID NOs: 11-27. In some embodiments, the linker comprises or consists of one or more of the amino acid sequences recited in SEQ ID NO: 16 or SEQ ID NO: 17.

In some embodiments, the fusion polypeptide comprises an N-terminal sequence. In some embodiments, the fusion polypeptide comprises an N-terminal sequence A. In some embodiments, the fusion polypeptide comprises a C-terminal sequence. In some embodiments, the fusion polypeptide comprises a C-terminal sequence E. In certain embodiments, the N-terminal or C-terminal sequence comprises one or more tags selected from the group consisting of a His-tag, a FLAG-tag, Arg-tag, T7-tag, Strep-tag, S-tag, an AviTag™, and an aptamer-tag. In some embodiments, the N-terminal or C-terminal sequence comprises a His-tag and an AviTag™. In some embodiments, the N-terminal or C-terminal sequence consists of a His-tag and an AviTag™. In some embodiments, the N-terminal or C-terminal sequence comprises a FLAG-tag, a His-tag, and an AviTag™. In some embodiments, the N-terminal or C-terminal sequence consists of a FLAG-tag, a His-tag, and an AviTag™. In certain embodiments, the N-terminal or C-terminal sequence comprises or consists of the amino acid sequence recited in SEQ ID NO: 31 or SEQ ID NO: 32.

In some embodiments, the N-terminal or C-terminal sequence comprises a moiety to increase the half-life of the fusion polypeptide in vivo. In certain embodiments, the N-terminal or C-terminal sequence comprises a PEG sequence, a PAS sequence, or an antibody sequence. In some embodiments, the N-terminal or C-terminal sequence comprises a moiety to increase the half-life of the fusion polypeptide in vivo, wherein the N-terminal or C-terminal sequence comprises a PEG sequence, a PAS sequence, or an antibody sequence, optionally selected from a Fab or ScFv molecule. In some embodiments, the antibody sequence is a Fab or ScFv molecule. In some embodiments, the antibody, Fab, or ScFv binds albumin. In certain embodiments, the antibody is CA645.

In some embodiments, the fusion polypeptide described herein comprises or consists of the amino acid sequence of any one of SEQ ID NOs: 33-40, 46-48, 51, 53, 54, or 55. In some embodiments, the fusion polypeptide described herein comprises or consists of any one of the amino acid sequences in Table 1, optionally without a signal peptide.

In other aspects, the disclosure relates to a nucleic acid molecule (e.g., an isolated nucleic acid molecule), including DNA and RNA molecules, that encodes a fusion polypeptide as described herein.

In some embodiments, the nucleic acid molecule comprises a nucleotide sequence having at least about 80%, at least about 85%, at least about 90%, at least about 95%, at least about 97%, or at least about 99% sequence identity with a nucleic acid encoding any one of SEQ ID NOS: 33-40, 46-48, 51, 53, 54, or 55. In some embodiments, the nucleic acid molecule comprises a nucleotide sequence having at least about 80%, at least about 85%, at least about 90%, at least about 95%, at least about 97%, or at least about 99% sequence identity with a nucleic acid encoding any one of the amino acid sequences in Table 1.

In some embodiments, the nucleic acid molecule comprises a nucleotide sequence having at least about 80%, at least about 85%, at least about 90%, at least about 95%, at least about 97% or at least about 99% sequence identity with SEQ ID NO: 44.

In some embodiments, the nucleic acid molecule comprises or consists of the nucleotide sequence of SEQ ID NO: 44.

Also disclosed are vectors, particularly expression vectors, comprising a nucleic acid molecule of the disclosure.

The disclosure also provides host cells comprising a nucleic acid and/or vector that encodes a fusion polypeptide as described herein.

The disclosure also provides a method for making a fusion polypeptide as described herein, comprising maintaining a host cell of the disclosure under conditions suitable for expression of the nucleic acid, whereby the recombinant nucleic acid is expressed and the fusion polypeptide is produced. The method may further comprise isolating and/or purifying the fusion polypeptide.

The disclosure also provides a pharmaceutical composition comprising a fusion polypeptide, nucleic acid molecule, vector or host cell of the disclosure, optionally further comprising a pharmaceutically or physiologically acceptable diluent and/or carrier. The disclosure also provides a pharmaceutical composition comprising a fusion polypeptide, nucleic acid molecule, vector, or host cell of the disclosure, and a pharmaceutically or physiologically acceptable diluent and/or carrier.

The disclosure also provides a method of treating a patient suffering from a pathological condition, comprising administering a fusion polypeptide, nucleic acid molecule, vector, host cell, or pharmaceutical composition of the disclosure to a subject in need thereof. In one embodiment, the subject may be suffering from chylomicronemia syndrome.

The disclosure also provides a fusion polypeptide, nucleic acid molecule, vector, host cell, or pharmaceutical composition according to the disclosure (i) for use in therapy and/or (ii) in the manufacture of a medicament for the treatment of a pathological disorder, disease, or condition disclosed herein. In some embodiments, provided herein is a method of treating a patient suffering from a pathological disorder, disease, or condition comprising administering a therapeutically effective amount of a fusion polypeptide, nucleic acid, vector, host cell, or pharmaceutical composition described herein to said patient. In some embodiments, the pathological disorder, disease, or condition may be selected from chylomicronemia (including Familial chylomicronemia syndrome, polygenic late-onset chylomicronemia and early-onset chylomicronemia), hyperlipidemic pancreatitis, hypertriglyceridemia, abdominal pain, recurrent acute pancreatitis, eruptive cutaneous xanthomata, lipemia retinalis, hepatosplenomegaly, diabetes, obesity, cardiovascular disease, chronic kidney disease, non-alcoholic fatty liver disease, hypertriglyceridemic pancreatitis, hepatosteatosis, metabolic syndrome, ischemic heart disease, and microvascular pathology. In one embodiment, the condition may be chylomicronemia syndrome (e.g., familial chylomicronemia syndrome).

### BRIEF DESCRIPTION OF THE FIGURES

FIG. 1 is a series of graphs showing the aggregation levels, yield, and lipase activity of various LPL constructs. FIG. 1, panels (A) and (C) demonstrate that LPL (A) was successfully expressed, but in an aggregated form (C). When LPL was co-expressed with GPIHBP1 (B) aggregation was not observed (C). Co-expression of LPL and GPIHBP1 resulted in a higher LPL activity (D) and also protected the LPL against spontaneous inactivation (E).

FIG. 2 is a series of graphs demonstrating the aggregation levels, activity, and stability of an LPL-GPIHBP1 fusion polypeptide. FIG. 1, panels (A) and (B) demonstrate that the LPL-GPIHBP1 fusion polypeptide was stably expressed (A) and was free of aggregation (B). The LPL-GPIHBP1 fusion polypeptide had comparable activity to the co-expressed construct (C) and was also stable (D).

FIG. 3 shows a schematic and results of an assay to test whether ANGPTL4 could displace LPL from the fusion polypeptide (A). ANGPTL4 was able to displace LPL from the co-expressed LPL/GPIHBP1 complex (B). Binding of ANGPTL4 to both the fusion polypeptide and co-expressed complex is demonstrated (C). Both ANGPTL4 and GPIHBP1 are able to dissociate the LPL/GPIHBP1 complex (D).

FIG. 4 is a set of graphs demonstrating resistance of the LPL/GPIHBP1 complex or LPL/GPIHBP1 fusion polypeptide to inactivation by ANGPTL4 (A) or ANGPTL3 (B).

FIG. 5 is a set of graphics that demonstrate the mapping of the ANGPTL4 binding epitope of LPL for both the LPL/GPIHBP1 fusion polypeptide (A) and co-expressed LPL/GPIHBP1 complex (B).

FIG. 6 is a set of graphs demonstrating the ability of the LPL/GPIHBP1 fusion polypeptide administered subcutaneously to lower TG levels in C57BL/6 mice.

FIG. 7 is a series of graphs that demonstrate the ability of the LPL/GPIHBP1 fusion polypeptide to lower TG levels in DBA/2 mice when administered intravenously (A, B). No increase in plasma free fatty acids was observed (C). Daily administration over 5 days also consistently decreased plasma TG (D, E).

FIG. 8 is a set of graphs that demonstrate the ability of the LPL/GPIHBP1 fusion polypeptide to lower TG levels in DBA/2 mice administered subcutaneously.

FIG. 9 is a series of graphs that demonstrate dose dependent lowering of TG levels by the LPL/GPIHBP1 fusion polypeptide administered subcutaneously in TALLYHO mice on a normal diet with a single dose (A, B) or a high fat diet with repeat dosing (C, D).

FIG. 10 is a set of graphs that demonstrate increased duration of TG lowering in LPL/GPIHBP1 fusion polypeptide linked to an albumin-binding moiety.

### DETAILED DESCRIPTION

### Lipoprotein Lipase (LPL) and Functional Variants Thereof

The LPL polypeptide used in the fusion polypeptide described herein may be mammalian such as, for example, human. Wild-type human LPL is encoded by the amino acid sequence having UniProtKB/Swiss-Prot accession no: P06858.1 (SEQ ID NO: 1). Human LPL is initially translated as a precursor protein having 475 amino acids. The signal peptide comprising amino acids 1-27 of this precursor protein are then cleaved, leaving the mature form comprising amino acids 28-475 (SEQ ID NO: 2).

In one embodiment, an LPL polypeptide used in the fusion polypeptide of the disclosure comprises an amino acid sequence having at least about 80%, at least about 85%, at least about 90%, at least about 95%, at least about 97%, at least about 98%, or at least about 99% sequence identity to the LPL polypeptide of SEQ ID NO: 1 or SEQ ID NO:2. In one embodiment, an LPL polypeptide used in the fusion polypeptide of the disclosure consists of an amino acid sequence having at least about 80%, at least about 85%, at least about 90%, at least about 95%, at least about 97%, at least about 98%, or at least about 99% sequence identity to the LPL polypeptide of SEQ ID NO: 1 or SEQ ID NO:2. In one embodiment the LPL polypeptide used in the fusion polypeptide of the disclosure comprises or consists of that of SEQ ID NO: 1. In one embodiment the LPL polypeptide used in the fusion polypeptide of the disclosure comprises or consists of that of SEQ ID NO: 2.

In another embodiment, a functional variant of an LPL polypeptide used in the fusion polypeptide of the disclosure may comprise one or more (e.g., 1, 2, 3, 4, 5, 6, 7, 8, 9, 10, or more) point mutations that add to, delete, or substitute any of the amino acids of the mature human LPL polypeptide (SEQ ID NO: 2).

As used herein, “functional variant” refers to a variant of a parent protein having substantial or significant sequence identity to the parent protein and retains at least one of the biological activities of the parent protein. A functional variant of a parent protein can be prepared by means known in the art in view of the present disclosure. A functional variant can include one or more modifications to the amino acid sequence of the parent protein. The modifications can change the physico-chemical properties of the polypeptide, for example, by improving the thermal stability of the polypeptide, altering the substrate specificity, changing the pH optimum, and the like. The modifications can also alter the biological activities of the parent protein, as long as they do not destroy or abolish all of the biological activities of the parent protein.

In some embodiments, a functional variant of a parent protein comprises a substitution, such as a conservative amino acid substitution, to the parent protein that does not significantly affect the biological activity of the parent protein. Conservative substitutions include, but are not limited to, amino acid substitutions within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Non-standard or non-natural amino acids (such as 4-hydroxyproline, 6-N-methyl lysine, 2-aminoisobutyric acid, isovaline, and alpha-methyl serine) can also be used to substitute standard amino acid residues in a parent protein.

In some embodiments, a functional variant of a parent protein comprises a deletion and/or insertion of one or more amino acids to the parent protein. For example, a functional variant of an LPL protein (e.g., a mature LPL protein) can include a deletion and/or insertion of 1, 2, 3, 4, 5, 6, 7, 8, 9, 10 or more amino acids to the LPL protein (e.g., the mature LPL protein)

In some embodiments, a functional variant of a parent protein comprises a substitution, such as a conservative amino acid substitution, and a deletion and/or insertion, such as a small deletion and/or insertion of amino acids, to the parent protein.

For example, in one embodiment, one or more point mutations may be made to the RAKR sequence found at amino acids 294-297 of SEQ ID NO: 2 (amino acids 321-324 of SEQ ID NO:1). In one embodiment the mutation in SEQ ID NO: 2 is R294A (as shown in SEQ ID NO: 45). The RAKR sequence is a cleavage site for proprotein convertase. As such, mutation of this site can make the polypeptide resistant to protein cleavage. Alternative mutations may be made to the LPL polypeptide used in the fusion polypeptide of the disclosure to resist proteolytic cleavage, and are encompassed within the scope of the disclosure.

Other point mutations may be made to improve the function of the LPL polypeptide. For example, in one embodiment, the functional variant LPL polypeptide used in the fusion polypeptide described herein may be the so-called “S447X” mutant version. This variant has a C to G change at nucleotide 1595 of the LPL nucleotide sequence. This leads to a change of Serine 447 to a stop codon, resulting in a truncated version of LPL lacking the last two C-terminal amino acids (S and G). This truncated version is shown in SEQ ID NO: 3. Thus, in one embodiment, the functional variant LPL polypeptide used in the disclosure comprises or consists of SEQ ID NO: 3.

The LPL polypeptide used in the fusion polypeptide of the disclosure may comprise one or more point mutations in the ANGPTL4 binding site. Thus, the LPL polypeptide used in the fusion polypeptide of the disclosure may comprise one or more point mutations (deletions, additions, or substitutions) in the region spanning amino acids 157-189 of SEQ ID NO: 1.

Other truncated versions of the LPL polypeptide may also be used in fusion polypeptides of the disclosure. For example, a truncated version of LPL comprising only amino acids 37-334 (SEQ ID NO: 4), referred to herein as the minimal LPL catalytic domain, may be used in the fusion polypeptides of the disclosure. Other truncated versions of LPL useful in the fusion polypeptides of the disclosure include those corresponding to the amino acid sequence of a polypeptide comprising or consisting of amino acids 36-335, 35-340, 34-345, 33-350, 32-355, 31-360, 30-365, 29-370, 28-375, 28-380, 28-385, 28-390, 28-395, 28-400, 28-405, 28-410, 28-415, 28-420, 28-425, 28-430, 28-435, 28-440, 28-445, 28-450, 28-455, 28-460, 28-465, or 28-470 (all with reference to SEQ ID NO: 1).

Other mutated versions or truncated versions of the wild type LPL polypeptide are also suitable for use in the fusion polypeptides of the disclosure. Any such mutated versions or truncated versions that are used as functional variants of LPL polypeptides in the disclosure should preferably retain at least a portion of the activity of the wild type mature polypeptide shown in SEQ ID NO: 2. In one embodiment, a functional variant of an LPL polypeptide of the disclosure retains at least 10% (for example 20%, 30%, 40%, 50%, 60%, 70%, 80%, 90%, 95%, 97%, 98%, 99% or more) of the activity of the wild type mature polypeptide shown in SEQ ID NO: 2. In a certain embodiment, the functional variant of an LPL polypeptide of the disclosure retains at least 90% or more of the activity of the wild type mature polypeptide shown in SEQ ID NO: 2. In one embodiment, the activity may be measured using a triolein lipase activity assay. See, for example, the HR Series NEFA-HR(2) assays from FUJIFILM Wako Diagnostics U.S.A. Corporation or Hoppe & Theimer, 1996, Phytochemistry, 42(4):973-978.

### Glycosylphosphatidylinositol HDL-Binding Protein 1 (GPIHBP1) and Functional Variants Thereof

The GPIHBP1 polypeptide used in the fusion polypeptide described herein may be mammalian including, for example, human. Wild-type human GPIHBP1 is encoded by the amino acid sequence having UniProtKB/Swiss-Prot accession no: Q8IV16 (SEQ ID NO: 5). Human GPIHBP1 is initially translated as a precursor protein, having 184 amino acids. The signal peptide comprising amino acids 1-20 of this precursor protein are then cleaved, resulting in a form comprising amino acids 21-184 (SEQ ID NO: 6). Human GPIHBP1 also comprises a propeptide when initially translated, which spans amino acids 152-184. This too is removed, resulting in a mature form comprising amino acids 21-151 (SEQ ID NO: 7).

In one embodiment, a GPIHBP1 polypeptide used in the fusion polypeptide of the disclosure comprises an amino acid sequence having at least about 80%, at least about 85%, at least about 90%, at least about 95%, at least about 97%, at least about 98%, or at least about 99% sequence identity to the GPIHBP1 polypeptide of SEQ ID NO: 5, SEQ ID NO: 6 or SEQ ID NO: 7. In one embodiment, a GPIHBP1 polypeptide used in the fusion polypeptide of the disclosure consists of an amino acid sequence having at least about 80%, at least about 85%, at least about 90%, at least about 95%, at least about 97%, at least about 98%, or at least about 99% sequence identity to the GPIHBP1 polypeptide of SEQ ID NO: 5, SEQ ID NO: 6 or SEQ ID NO: 7. In one embodiment the GPIHBP1 polypeptide used in the fusion polypeptide of the disclosure comprises or consists of that of SEQ ID NO: 5. In one embodiment the GPIHBP1 polypeptide used in the fusion polypeptide of the disclosure comprises or consists of that of SEQ ID NO: 6. In one embodiment the GPIHBP1 polypeptide used in the fusion polypeptide of the disclosure comprises or consists of that of SEQ ID NO: 7.

In another embodiment, a functional variant GPIHBP1 polypeptide used in the fusion polypeptide of the disclosure may comprise one or more (e.g., 1, 2, 3, 4, 5, 6, 7, 8, 9, 10 or more) point mutations that add to, delete, or substitute any of the amino acids of the mature human GPIHBP1 polypeptide (SEQ ID NO: 7).

As used herein, “functional variant” refers to a variant of a parent protein having substantial or significant sequence identity to the parent protein and retains at least one of the biological activities of the parent protein. A functional variant of a parent protein can be prepared by means known in the art in view of the present disclosure. A functional variant can include one or more modifications to the amino acid sequence of the parent protein. The modifications can change the physico-chemical properties of the polypeptide, for example, by improving the thermal stability of the polypeptide, altering the substrate specificity, changing the pH optimum, and the like. The modifications can also alter the biological activities of the parent protein, as long as they do not destroy or abolish all of the biological activities of the parent protein.

In some embodiments, a functional variant of a parent protein comprises a substitution, such as a conservative amino acid substitution, to the parent protein that does not significantly affect the biological activity of the parent protein. Conservative substitutions include, but are not limited to, amino acid substitutions within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Non-standard or non-natural amino acids (such as 4-hydroxyproline, 6-N-methyl lysine, 2-aminoisobutyric acid, isovaline, and alpha-methyl serine) can also be used to substitute standard amino acid residues in a parent protein.

In some embodiments, a functional variant of a parent protein comprises a deletion and/or insertion of one or more amino acids to the parent protein. For example, a functional variant of an GPIHBP1 protein (e.g., a mature GPIHBP1 protein) can include a deletion and/or insertion of 1, 2, 3, 4, 5, 6, 7, 8, 9, 10 or more amino acids to the GPIHBP1 protein (e.g., the mature GPIHBP1 protein)

In some embodiments, a functional variant of a parent protein comprises a substitution, such as a conservative amino acid substitution, and a deletion and/or insertion, such as a small deletion and/or insertion of amino acids, to the parent protein.

Truncated versions of the GPIHBP1 polypeptide may also be used in fusion polypeptides of the disclosure. For example, a truncated version of the GPIHBP1 polypeptide, lacking the propeptide, but retaining the signal peptide, may comprise amino acids 1-151 of SEQ ID NO: 5 (SEQ ID NO: 8). An alternative truncated version of GPIHBP1 comprising only amino acids 63-148 (SEQ ID NO: 9), referred to herein as the minimal functional domain of human GPIHBP1, may be used in the fusion polypeptides of the disclosure. Other truncated versions of GPIHBP1 useful in the fusion polypeptides of the disclosure include those comprising amino acids 62-149, 61-150, 60-151, 55-152, 50-153, 45-154, 40-155, 35-156, 30-157, 25-158, 21-160, 20-159 (all with reference to SEQ ID NO: 5). The truncated version comprising amino acids 21-160 is referred to herein as SEQ ID NO: 10.

Other mutated versions or truncated versions of the wild type GPIHBP1 polypeptide are also suitable for use in the fusion polypeptides of the disclosure. Any such mutated versions or truncated versions that are used as functional variants of GPIHBP1 polypeptides in the fusion polypeptides of the disclosure should preferably retain the activity of the wild type mature polypeptide shown in SEQ ID NO: 7. In one embodiment, a functional variant of an GPIHBP1 polypeptide of the disclosure retains at least 10% (for example 20%, 30%, 40%, 50%, 60%, 70%, 80%, 90%, 95%, 97%, 98%, 99% or more) of the activity of the wild type mature polypeptide shown in SEQ ID NO: 7. In a certain embodiment, the functional variant of an GPIHBP1 polypeptide of the disclosure retains at least 90% or more of the activity of the wild type mature polypeptide shown in SEQ ID NO: 7. The activity of functional variants of GPIHBP1 may be measured by assessing the ability of said functional variants to protect LPL from spontaneous inactivation and from inactivation by ANGPTL3 and ANGPTL4.

### Exemplary Combinations of LPL and GPIHBP1

The fusion polypeptides of the disclosure may comprise combinations of any of the LPL and GPIHBP1 moieties described in this disclosure. As a non-limiting set of examples, the LPL moiety may comprise or consist of any one of SEQ ID NOs: 1, 2, 3, 4, or 45, and the GPIHBP1 moiety may comprise or consist of any one of SEQ ID NOs: 5, 6, 7, 8, 9 or 10.

In one embodiment, the fusion protein may comprise an LPL moiety of SEQ ID NO: 1, and a GPIHBP1 moiety of SEQ ID NO: 5. In one embodiment, the fusion protein may comprise an LPL moiety of SEQ ID NO: 1, and a GPIHBP1 moiety of SEQ ID NO: 6. In one embodiment, the fusion protein may comprise an LPL moiety of SEQ ID NO: 1, and a GPIHBP1 moiety of SEQ ID NO: 7. In one embodiment, the fusion protein may comprise an LPL moiety of SEQ ID NO: 1, and a GPIHBP1 moiety of SEQ ID NO: 8. In one embodiment, the fusion protein may comprise an LPL moiety of SEQ ID NO: 1, and a GPIHBP1 moiety of SEQ ID NO: 9. In one embodiment, the fusion protein may comprise an LPL moiety of SEQ ID NO: 1, and a GPIHBP1 moiety of SEQ ID NO: 10.

In one embodiment, the fusion protein may comprise an LPL moiety of SEQ ID NO: 2, and a GPIHBP1 moiety of SEQ ID NO: 5. In one embodiment, the fusion protein may comprise an LPL moiety of SEQ ID NO: 2, and a GPIHBP1 moiety of SEQ ID NO: 6. In one embodiment, the fusion protein may comprise an LPL moiety of SEQ ID NO: 2, and a GPIHBP1 moiety of SEQ ID NO: 7. In one embodiment, the fusion protein may comprise an LPL moiety of SEQ ID NO: 2, and a GPIHBP1 moiety of SEQ ID NO: 8. In one embodiment, the fusion protein may comprise an LPL moiety of SEQ ID NO: 2, and a GPIHBP1 moiety of SEQ ID NO: 9. In one embodiment, the fusion protein may comprise an LPL moiety of SEQ ID NO: 2, and a GPIHBP1 moiety of SEQ ID NO: 10.

In one embodiment, the fusion protein may comprise an LPL moiety of SEQ ID NO: 3, and a GPIHBP1 moiety of SEQ ID NO: 5. In one embodiment, the fusion protein may comprise an LPL moiety of SEQ ID NO: 3, and a GPIHBP1 moiety of SEQ ID NO: 6. In one embodiment, the fusion protein may comprise an LPL moiety of SEQ ID NO: 3, and a GPIHBP1 moiety of SEQ ID NO: 7. In one embodiment, the fusion protein may comprise an LPL moiety of SEQ ID NO: 3, and a GPIHBP1 moiety of SEQ ID NO: 8. In one embodiment, the fusion protein may comprise an LPL moiety of SEQ ID NO: 3, and a GPIHBP1 moiety of SEQ ID NO: 9. In one embodiment, the fusion protein may comprise an LPL moiety of SEQ ID NO: 3, and a GPIHBP1 moiety of SEQ ID NO: 10.

In one embodiment, the fusion protein may comprise an LPL moiety of SEQ ID NO: 4, and a GPIHBP1 moiety of SEQ ID NO: 5. In one embodiment, the fusion protein may comprise an LPL moiety of SEQ ID NO: 4, and a GPIHBP1 moiety of SEQ ID NO: 6. In one embodiment, the fusion protein may comprise an LPL moiety of SEQ ID NO: 4, and a GPIHBP1 moiety of SEQ ID NO: 7. In one embodiment, the fusion protein may comprise an LPL moiety of SEQ ID NO: 4, and a GPIHBP1 moiety of SEQ ID NO: 8. In one embodiment, the fusion protein may comprise an LPL moiety of SEQ ID NO: 4, and a GPIHBP1 moiety of SEQ ID NO: 9. In one embodiment, the fusion protein may comprise an LPL moiety of SEQ ID NO: 4, and a GPIHBP1 moiety of SEQ ID NO: 10.

In one embodiment, the fusion protein may comprise an LPL moiety of SEQ ID NO: 45, and a GPIHBP1 moiety of SEQ ID NO: 5. In one embodiment, the fusion protein may comprise an LPL moiety of SEQ ID NO: 45, and a GPIHBP1 moiety of SEQ ID NO: 6. In one embodiment, the fusion protein may comprise an LPL moiety of SEQ ID NO: 45, and a GPIHBP1 moiety of SEQ ID NO: 7. In one embodiment, the fusion protein may comprise an LPL moiety of SEQ ID NO: 45, and a GPIHBP1 moiety of SEQ ID NO: 8. In one embodiment, the fusion protein may comprise an LPL moiety of SEQ ID NO: 45, and a GPIHBP1 moiety of SEQ ID NO: 9. In one embodiment, the fusion protein may comprise an LPL moiety of SEQ ID NO: 45, and a GPIHBP1 moiety of SEQ ID NO: 10.

### Linkers

The LPL and GPIHBP1 moieties described in this disclosure can be directly bonded to each other in a contiguous polypeptide chain, or are indirectly bonded to each other through a suitable linker. The linker may be a peptide linker. Peptide linkers are commonly used in fusion polypeptides and methods for selecting or designing linkers are well-known. (See, e.g., Chen X et al. Adv. Drug Deliv. Rev. 65(10):135701369 (2013) and Wriggers W et al., Biopolymers 80:736-746 (2005), the contents of each of which are incorporated herein for this purpose).

Peptide linkers generally are categorized as i) flexible linkers, ii) helix forming linkers, and iii) cleavable linkers, and examples of each type are known in the art. In one example, a flexible linker is included in the fusion polypeptides described herein. Flexible linkers may contain a majority of amino acids that are sterically unhindered, such as glycine and alanine. The hydrophilic amino acid Ser is also conventionally used in flexible linkers. Examples of flexible linkers include, without limitation: polyglycines (e.g., (Gly)₄ and (Gly)₅), polyalanines poly(Gly-Ala), and poly(Gly-Ser) (e.g., (Glyₙ-Serₙ)ₙ or (Serₙ-Glyₙ)ₙ, wherein each n is independently an integer equal to or greater than 1).

Peptide linkers can be of a suitable length. The peptide linker sequence may be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues in length. For example, a peptide linker can be from about 5 to about 50 amino acids in length; from about 10 to about 40 amino acids in length; from about 15 to about 30 amino acids in length; or from about 15 to about 20 amino acids in length. Variation in peptide linker length may retain or enhance activity, giving rise to superior efficacy in activity studies. The peptide linker sequence may be comprised of naturally or non-naturally occurring amino acids, or a mixture of both naturally and non-naturally occurring amino acids.

In some aspects, the amino acids glycine and serine comprise the amino acids within the linker sequence. In certain aspects, the linker region comprises sets of glycine repeats (GSG₃)ₙ (SEQ ID NO: 11), where n is a positive integer equal to or greater than 1 (for example 1 to about 20). More specifically, the linker sequence may be GSGGG (SEQ ID NO: 12). The linker sequence may be GSGG (SEQ ID NO: 13). In certain other aspects, the linker region orientation comprises sets of glycine repeats (SerGly₃)ₙ, where n is a positive integer equal to or greater than 1 (for example, 1 to about 20) (SEQ ID NO: 14).

In other embodiments, a linker may contain glycine (G) and serine (S) in a random or a repeated pattern. For example, the linker can be (GGGGS)ₙ (SEQ ID NO: 15), wherein n is an integer ranging from 1 to 20, for example 1 to 4. In a particular example, n is 4 and the linker is GGGGSGGGGSGGGGSGGGGS (SEQ ID NO: 16). In another particular example, n is 3 and the linker is GGGGSGGGGSGGGGS (SEQ ID NO: 17).

In other embodiments, a linker may contain glycine (G), serine (S) and proline (P) in a random or repeated pattern. For example, the linker can be (GPPGS)ₙ, wherein n is an integer ranging from 1 to 20, for example 1-4. In a particular example, n is 1 and the linker is GPPGS (SEQ ID NO: 18).

In general, the linker is not immunogenic when administered in a patient or subject, such as a human. Thus linkers may be chosen such that they have low immunogenicity or are thought to have low immunogenicity.

The linkers described herein are exemplary, and the linker can include other amino acids, such as Glu and Lys, if desired. The peptide linkers may include multiple repeats of, for example, (G3S) (SEQ ID NO: 19), (G45) (SEQ ID NO: 15), (GYS) (SEQ ID NO: 20), and/or (GlySer) (SEQ ID NO: 21), if desired. In certain aspects, the peptide linkers may include multiple repeats of, for example, (SG₄) (SEQ ID NO: 22), (SG₃) (SEQ ID NO: 14), (SG₂) (SEQ ID NO: 23) or (SerGly) (SEQ ID NO: 24).

In other aspects, the peptide linkers may include combinations and multiples of repeating amino acid sequence units, such as (G3S)+(G4S)+(GlySer) (SEQ ID NO: 19+SEQ ID NO: 15+SEQ ID NO: 21). In other aspects, Ser can be replaced with Ala (e.g., (G4A) (SEQ ID NO: 25) or (G3A) (SEQ ID NO: 26)). In yet other aspects, the linker comprises the motif (EAAAK)ₙ, where n is a positive integer equal to or greater than 1, for example 1 to about 20 (SEQ ID NO: 27). In certain aspects, peptide linkers may also include cleavable linkers.

### N-Terminal Sequences & C-Terminal Sequences

Various sequences may be attached to the N- or C-terminus of the fusion polypeptides of the disclosure. These may be functional, such as signal peptides, purification tags/sequences, or half-life extension moieties, or may simply comprise spacer sequences. In some embodiments, the signal peptide may be METDTLLLWVLLLWVPGSTG (SEQ ID NO: 52). Any of the fusion polypeptides described herein may be used with or without such a signal peptide.

### Purification Tags and Markers

A variety of tags or markers may be attached to the N- or C-terminus of the fusion polypeptides of the disclosure to assist with purification. Any affinity tag may be combined with the fusion polypeptides of the disclosure to assist with purification. Examples of such affinity tags are a His-tag, a FLAG-tag, Arg-tag, T7-tag, Strep-tag, S-tag, aptamer-tag, AviTag™, or any combination of these tags. In one embodiment the affinity tag is a His-tag (usually comprising 5-10 histidine residues), for example a 6His tag (i.e., HHHHHH) (SEQ ID NO: 28). In another embodiment, the affinity tag is a FLAG tag (i.e., DYKDDDDK) (SEQ ID NO: 29). In another embodiment, the affinity tag is an AviTag™ (i.e., GLNDIFEAQKIEWHE) (SEQ ID NO: 30). Various other tags for use in the disclosure are well known in the art.

Combinations of such affinity tags may also be used, either comprising one or more tags at the N-terminus, one or more tags at the C-terminus, or one or more tags at each of the N-terminus and the C-terminus. Examples of such combinations include a His tag (H) combined with an AviTag (A), or a His tag (H) combined with both an AviTag (A) and a FLAG tag (F). The tags may be in either orientation, thus the AviTag/His tag may have the orientation N-AH-C or N-HA-C, while the Avi/His/FLAG tag may have the orientation N-AHF-C, N-FHA-C, etc.

In one embodiment, a fusion polypeptide according to the disclosure comprises an “AHF” tag having the sequence “GLNDIFEAQKIEWHEGGHHHHHHDYKDDDDK” (SEQ ID NO: 31). In another embodiment, a fusion polypeptide according to the disclosure comprises an “FHA” tag having the sequence “DYKDDDDKHHHHHHGGGLNDIFEAQKIEWHE” (SEQ ID NO: 32). Any of the fusion polypeptides described herein may be used with or without such a tag (e.g., an AHF or FHA tag).

Any of the fusion polypeptides described herein may be used with or without such tags or markers.

### Half-Life Extension

In other embodiments, the fusion polypeptides of the disclosure may be modified at the N- or C-terminus to increase the half-life of the fusion polypeptides in vivo.

A variety of strategies can be used to extend the half-life of the fusion polypeptides. For example, the half-life may be extended by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; and/or by incorporation into nanocarriers, slow release formulations, or medical devices.

To prolong the serum circulation of fusion polypeptides in vivo, inert polymer molecules such as high molecular weight PEG can be attached to the fusion polypeptides described herein with or without a multifunctional linker either through site-specific conjugation of the PEG to the N- or C-terminus of the fusion polypeptides or via epsilon-amino groups present on lysine residues. To pegylate a fusion polypeptide, the fusion polypeptide is typically reacted with polyethylene glycol (PEG), such as a reactive ester or aldehyde derivative of PEG, under conditions in which one or more PEG groups become attached to the fusion polypeptide. The pegylation can be carried out by an acylation reaction or an alkylation reaction with a reactive PEG molecule (or an analogous reactive water-soluble polymer). As used herein, the term “polyethylene glycol” is intended to encompass any one of the forms of PEG that have been used to derivatize other proteins, such as mono (C1-C10)alkoxy- or aryloxy-polyethylene glycol or polyethylene glycol-maleimide. In one embodiment, the fusion polypeptide to be pegylated is aglycosylated. Linear or branched polymer derivatization that results in minimal loss of biological activity can be used. The degree of conjugation can be closely monitored by SDS-PAGE and mass spectrometry to ensure proper conjugation of PEG molecules to the fusion polypeptides. Unreacted PEG can be separated from fusion polypeptide-PEG conjugates by size-exclusion or by ion-exchange chromatography. PEG-derivatized fusion polypeptides can be tested for activity as well as for in vivo efficacy using methods well-known to those of skill in the art including, for example, by immunoassays. Methods for pegylating proteins are known in the art and can be applied to the fusion polypeptides disclosure herein, see for example, EP0154316 and EP0401384, the contents of each of which are incorporated herein for this purpose.

Other modified pegylation technologies may be used with fusion polypeptides described herein. In one embodiment, a fusion polypeptide described herein may include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a normative amino acid any place an amber codon is positioned, converting the amber from a stop codon to one that signals incorporation of the chemically specified amino acid.

In one embodiment, a fusion polypeptide described herein may comprise recombinant pegylation technology (rPEG) for serum half-life extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum half-life of the protein is greatly increased. In contrast to traditional PEGylation, which requires chemical conjugation and repurification, the manufacturing process is greatly simplified and the product is homogeneous.

An alternative to PEGylation is PASylation® (Schlapschy et al., 2013, Protein Eng Des Sel. 26(8):489-501, the contents of which are incorporated herein for this purpose). PASylation® is a polypeptide-based, random-coil domain (RCD) technology (see, e.g., WO2011/144756 and WO2008/155134, the contents of each of which are incorporated herein for this purpose). Thus, in one embodiment, a fusion polypeptide described herein may be PASylated. The polypeptides of PASylation® contain sequences of the amino acids proline, alanine, and optionally serine (PA/S, or PAS to indicate that serine is present). The polymer which is a combination of amino acid residues results in cancellation of the distinct secondary structure preferences of each amino acid residue to form a stably disordered polypeptide. Biologically active proteins attached to at least one PAS polypeptide, which contains a domain with an amino acid sequence that assumes a random coil conformation, have been observed to have increased in vivo and/or in vitro stability compared to the protein in its native state lacking this adduct.

PASylation® is said to provide certain advantages over PEGylation. For example, it maintains high target affinity; it has not elicited immunogenicity in preclinical trials to date; it is biodegradable such that it is efficiently degraded by kidney enzymes; and it is stable in the blood stream. PAS polypeptides show no polydispersity and does not require in vitro coupling steps, thereby not negatively affecting the cost of goods factor. The PAS polypeptide has lower viscosity for the comparable molecular weight of PEG and the half-life extension is tunable from 10-fold to greater than 300-fold. These advantages may render the protein modified by PASylation® more efficacious, safer, and considerably more convenient by way of lowered dosing and frequency of administration bringing about an increase in patient compliance.

The hydrodynamic volumes of the PAS200 polypeptide chain roughly corresponds to a PEG polymer of molecular weight 20 kDa, while that of the PAS600 polypeptide chain roughly corresponds to a PEG polymer of molecular weight 40 kDa. The data provide that for corresponding hydrodynamic volumes at the higher concentrations, the PAS polypeptides have viscosities that are one-third to three-fold lower than the PEG polymers.

Polysialylation uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body. In one embodiment a fusion polypeptide described herein may be polysialylated.

Another technology includes the use of hydroxyethyl starch (“HES”) derivatives linked to fusion polypeptides. In one embodiment, a fusion polypeptide disclosed herein may be hesylated. HES is a modified natural polymer derived from waxy maize starch and can be metabolized by the body's enzymes. HES solutions are usually administered to substitute deficient blood volume and to improve the rheological properties of the blood. Hesylation of a fusion polypeptide enables the prolongation of the circulation half-life by increasing the stability of the molecule, as well as by reducing renal clearance, resulting in an increased biological activity. By varying different parameters, such as the molecular weight of HES, a wide range of HES conjugates can be customized.

In one embodiment, the fusion polypeptides of the disclosure may be fused to one or more human serum albumin (HSA) polypeptides, or a portion thereof. The use of albumin as a component of an albumin fusion polypeptide as a carrier for various proteins has been described in WO93/15199, WO93/15200, and EP0413622. The use of N-terminal fragments of HSA for fusions to polypeptides has also been proposed (EP0399666). Accordingly, by genetically or chemically fusing or conjugating the fusion polypeptides of the disclosure to albumin, one can stabilize or extend the shelf-life, and/or to retain the molecule's activity for extended periods of time in solution, in vitro and/or in vivo. Additional methods pertaining to HSA fusions can be found, for example, in WO2001/077137 and WO2003/006007, the contents of each of which are incorporated herein for this purpose.

In one embodiment, the fusion polypeptides of the present disclosure can be fused to an antibody or antibody fragment thereof that binds to albumin, e.g., human serum albumin (HSA). As a non-limiting set of examples, the albumin-binding antibody or antibody fragment thereof can be a Fab, a scFv, a Fv, an scFab, a (Fab′)2, a single domain antibody, a camelid VHH domain, a VH or VL domain, or a full-length monoclonal antibody (mAb).

In one embodiment, the fusion polypeptides of the present disclosure may be fused to a fatty acid to extend their half-life. Fatty acids suitable for linking to a biomolecule have been described in the art, e.g., WO2015/200078, WO2015/191781, US2013/0040884, the contents of each of which are incorporated herein for this purpose. Suitable half-life extending fatty acids include those defined as a C6-70alkyl, a C6-70alkenyl or a C6-70alkynyl chain, each of which is substituted with at least one carboxylic acid (for example 1, 2, 3 or 4 CO₂H) and optionally further substituted with hydroxyl group. For example, the protein described herein can be linked to a fatty acid having any of the following Formulae A1, A2 or A3:

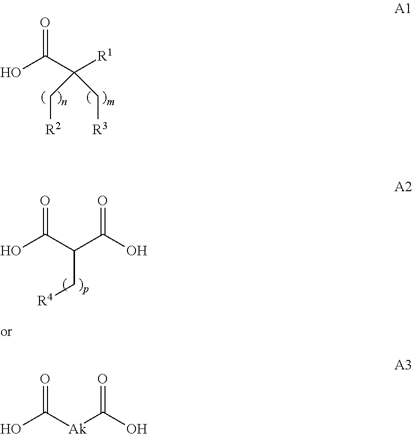

wherein R¹ is CO₂H or H;

R², R³ and R⁴ are independently of each other H, OH, CO₂H, —CH═CH₂ or —C≡CH;

Ak is a branched C₆-C₃₀ alkylene;

n, m and p are independently of each other an integer between 6 and 30; or an amide, ester or pharmaceutically acceptable salt thereof.

In some embodiments, the fatty acid is of Formula A1, e.g., a fatty acid of Formula A1 wherein n and m are independently 8 to 20, for example 10 to 16. In another embodiment, the fatty acid moiety is of Formula A1 and wherein at least one of R² and R³ is CO₂H.

In some embodiments, the fatty acid is selected from the following Formulae:

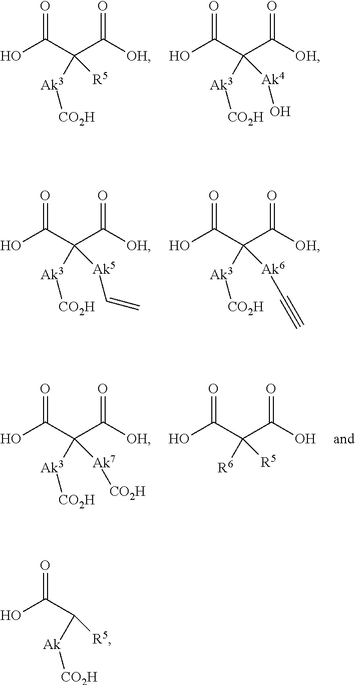

wherein Ak³, Ak⁴, Ak⁵, Ak⁶ and Ak⁷ are independently a (C₈₋₂₀)alkylene, and R⁵ and R⁶ are independently (C₈₋₂₀)alkyl.

In some embodiments, the fatty acid is selected from the following Formulae:

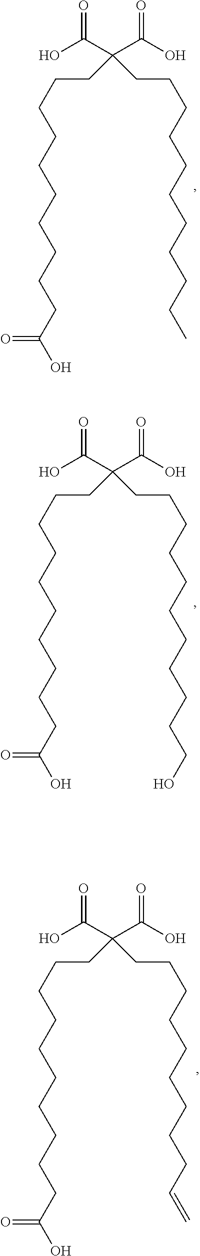 [SMILES: CCCCCCCCCCCC(CCCCCCCCCCC(=O)O)(C(=O)O)C(=O)O ; O=C(O)CCCCCCCCCCC(CCCCCCCCCCCO)(C(=O)O)C(=O)O ; C=CCCCCCCCCCC(CCCCCCCCCCC(=O)O)(C(=O)O)C(=O)O]

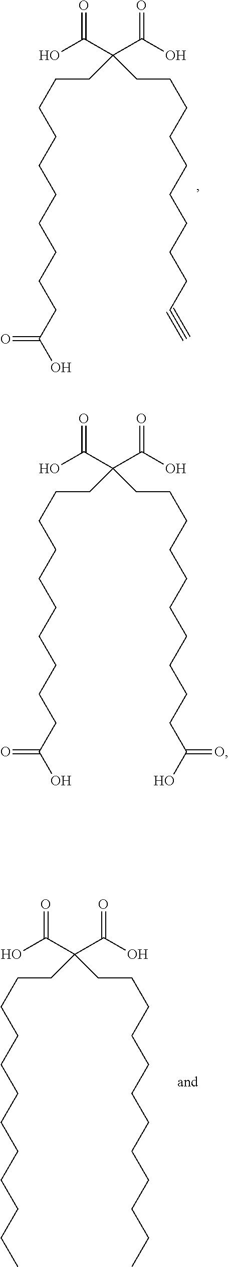 [SMILES: C#CCCCCCCCCCC(CCCCCCCCCCC(=O)O)(C(=O)O)C(=O)O ; O=C(O)CCCCCCCCCCC(CCCCCCCCCCC(=O)O)(C(=O)O)C(=O)O ; CCCCCCCCCCCCC(CCCCCCCCCCCC)(C(=O)O)C(=O)O]

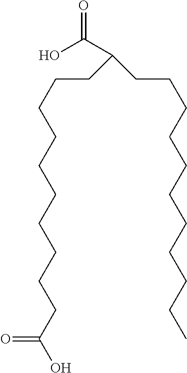 [SMILES: CCCCCCCCCCCC(CCCCCCCCCCC(=O)O)C(=O)O]

In some embodiments, the fatty acid is selected from the following Formulae:

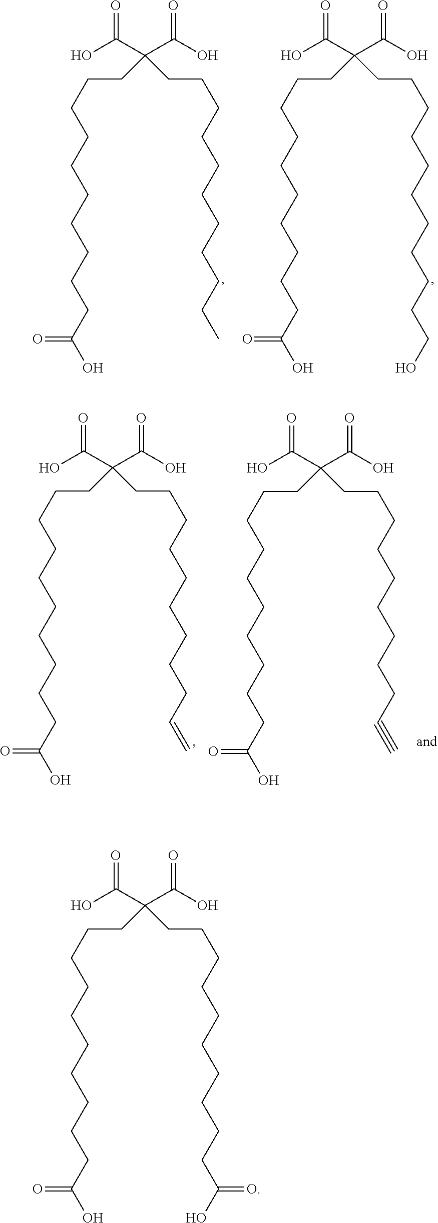

In some embodiments, the fatty acid is of Formula A2 or A3. In a particular embodiment, the conjugate comprises a fatty acid moeity of Formula A2 wherein p is 8 to 20, or a fatty acid moeity of Formula A3 wherein Ak is C₈₋₂₀alkylene.

Methods of increasing half-life of the fusion polypeptides of the disclosure include PASylation® and fusion to albumin binding antibodies. In one embodiment the albumin binding antibody may bind to human serum albumin. In one embodiment the albumin binding antibody is the Fab CA645 (Adams et al., 2016, MAbs, 8(7):1336-1346, the contents of which are incorporated herein for this purpose). In another embodiment, the albumin binding antibody is a CA645 ScFV. In one embodiment, the fusion polypeptide of the disclosure comprises PAS200 or PAS600.

### Exemplary Constructs

The present disclosure provides the following exemplary fusion polypeptide constructs in Table 1. The fusion polypeptides described in Table 1 may be used with or without a signal peptide (e.g., METDTLLLWVLLLWVPGSTG (SEQ ID NO: 52)). The fusion polypeptides described in Table 1 may be used with or without a tag (e.g., an AHF tag such as GLNDIFEAQKIEWHEGGHHHHHHDYKDDDDK (SEQ ID NO: 31) or an FHA such as DYKDDDDKHHHHHHGGGLNDIFEAQKIEWHE (SEQ ID NO: 32)). In some instances, the fusion polypeptides described in Table 1 may be used with or without both a signal peptide and a tag.

TABLE 1
Exemplary fusion polypeptide constructs

| Name | Sequence |
|---|---|
| SIGNAL peptide-AHF-hLPL(28- | METDTLLLWVLLLWVPGSTGGLNDIFEAQKIEWHEGGHHHH |
| 475)-(GGGGS)₄-hGPIHBP1(21- | HHDYKDDDDKADQRRDFIDIESKFALRTPEDTAEDTCHLIPG |
| 151) | VAESVATCHFNHSSKTFMVIHGWTVTGMYESWVPKLVAALY |
| (SEQ ID NO: 33) | KREPDSNVIVVDWLSRAQEHYPVSAGYTKLVGQDVARFINW |
| | MEEEFNYPLDNVHLLGYSLGAHAAGIAGSLTNKKVNRITGLDP |
| | AGPNFEYAEAPSRLSPDDADFVDVLHTFTRGSPGRSIGIQKPV |
| | GHVDIYPNGGTFQPGCNIGEAIRVIAERGLGDVDQLVKCSHE |
| | RSIHLFIDSLLNEENPSKAYRCSSKEAFEKGLCLSCRKNRCNN |
| | LGYEINKVRAKRSSKMYLKTRSQMPYKVFHYQVKIHFSGTES |
| | ETHTNQAFEISLYGTVAESENIPFTLPEVSTNKTYSFLIYTEVDI |
| | GELLMLKLKWKSDSYFSWSDWWSSPGFAIQKIRVKAGETQK |
| | KVIFCSREKVSHLQKGKAPAVFVKCHDKSLNKKSGGGGGSG |
| | GGGSGGGGSGGGGSQTQQEEEEEDEDHGPDDYDEEDEDE |
| | VEEEETNRLPGGRSRVLLRCYTCKSLPRDERCNLTQNCSHGQ |
| | TCTTLIAHGNTESGLLTTHSTWCTDSCQPITKTVEGTQVTMT |
| | CCQSSLCNVPPWQSSRVQDPTG |
| AHF-hLPL(28-475)-(GGGGS)₄- | GLNDIFEAQKIEWHEGGHHHHHHDYKDDDDKADQRRDFIDI |
| hGPIHBP1(21-151) (without | ESKFALRTPEDTAEDTCHLIPGVAESVATCHFNHSSKTFMVIH |
| signal sequence) | GWTVTGMYESWVPKLVAALYKREPDSNVIVVDWLSRAQEHY |
| (SEQ ID NO: 34) | PVSAGYTKLVGQDVARFINWMEEEFNYPLDNVHLLGYSLGAH |
| | AAGIAGSLTNKKVNRITGLDPAGPNFEYAEAPSRLSPDDADFV |
| | DVLHTFTRGSPGRSIGIQKPVGHVDIYPNGGTFQPGCNIGEAI |
| | RVIAERGLGDVDQLVKCSHERSIHLFIDSLLNEENPSKAYRCS |
| | SKEAFEKGLCLSCRKNRCNNLGYEINKVRAKRSSKMYLKTRS |
| | QMPYKVFHYQVKIHFSGTESETHTNQAFEISLYGTVAESENIP |
| | FTLPEVSTNKTYSFLIYTEVDIGELLMLKLKWKSDSYFSWSDW |
| | WSSPGFAIQKIRVKAGETQKKVIFCSREKVSHLQKGKAPAVF |
| | VKCHDKSLN KKSGGGGGSGGGGSGGGGSGGGGSQTQQEE |
| | EEEDEDHGPDDYDEEDEDEVEEEETNRLPGGRSRVLLRCYTC |
| | KSLPRDERCNLTQNCSHGQTCTTLIAHGNTESGLLTTHSTWC |
| | TDSCQPITKTVEGTQVTMTCCQSSLCNVPPWQSSRVQDPTG |
| hLPL(28-475)-(G4S)4- | ADQRRDFIDIESKFALRTPEDTAEDTCHLIPGVAESVATCHFN |
| hGPIHBP1(21-151) | HSSKTFMVIHGWTVTGMYESWVPKLVAALYKREPDSNVIVV |
| (SEQ ID NO: 55) | DWLSRAQEHYPVSAGYTKLVGQDVARFINWMEEEFNYPLDN |
| | VHLLGYSLGAHAAGIAGSLTNKKVNRITGLDPAGPNFEYAEAP |
| | SRLSPDDADFVDVLHTFTRGSPGRSIGIQKPVGHVDIYPNGG |
| | TFQPGCNIGEAIRVIAERGLGDVDQLVKCSHERSIHLFIDSLL |
| | NEENPSKAYRCSSKEAFEKGLCLSCRKNRCNNLGYEINKVRA |
| | KRSSKMYLKTRSQMPYKVFHYQVKIHFSGTESETHTNQAFEI |
| | SLYGTVAESENIPFTLPEVSTNKTYSFLIYTEVDIGELLMLKLK |
| | WKSDSYFSWSDWWSSPGFAIQKIRVKAGETQKKVIFCSREK |
| | VSHLQKGKAPAVFVKCHDKSLNKKSGGGGGSGGGGSGGGG |
| | SGGGGSQTQQEEEEEDEDHGPDDYDEEDEDEVEEEETNRLP |
| | GGRSRVLLRCYTCKSLPRDERCNLTQNCSHGQTCTTLIAHGN |
| | TESGLLTTHSTWCTDSCQPITKTVEGTQVTMTCCQSSLCNVP |
| | PWQSSRVQDPTG |
| SIGNAL peptide-AHF-hLPL(28- | METDTLLLWVLLLWVPGSTGGLNDIFEAQKIEWHEGGHHHH |
| 475)-(G4S)4-hGPIHBP1(21- | HHDYKDDDDKADQRRDFIDIESKFALRTPEDTAEDTCHLIPG |
| 151)-(G4S)3-PAS200 | VAESVATCHFNHSSKTFMVIHGWTVTGMYESWVPKLVAALY |
| (SEQ ID NO: 35) | KREPDSNVIVVDWLSRAQEHYPVSAGYTKLVGQDVARFINW |
| | MEEEFNYPLDNVHLLGYSLGAHAAGIAGSLTNKKVNRITGLDP |
| | AGPNFEYAEAPSRLSPDDADFVDVLHTFTRGSPGRSIGIQKPV |
| | GHVDIYPNGGTFQPGCNIGEAIRVIAERGLGDVDQLVKCSHE |
| | RSIHLFIDSLLNEENPSKAYRCSSKEAFEKGLCLSCRKNRCNN |
| | LGYEINKVRAKASSKMYLKTRSQMPYKVFHYQVKIHFSGTES |
| | ETHTNQAFEISLYGTVAESENIPFTLPEVSTNKTYSFLIYTEVDI |
| | GELLMLKLKWKSDSYFSWSDWWSSPGFAIQKIRVKAGETQK |
| | KVIFCSREKVSHLQKGKAPAVFVKCHDKSLNKKSGGGGGSG |
| | GGGSGGGGSGGGGSQTQQEEEEEDEDHGPDDYDEEDEDE |
| | VEEEETNRLPGGRSRVLLRCYTCKSLPRDERCNLTQNCSHGQ |
| | TCTTLIAHGNTESGLLTTHSTWCTDSCQPITKTVEGTQVTMT |
| | CCQSSLCNVPPWQSSRVQDPTGGGGGSGGGGSGGGGSSS |
| | AAASSSASPAAPAPASPAAPAPSAPAASPAAPAPASPAAPAPS |
| | APAASPAAPAPASPAAPAPSAPAASPAAPAPASPAAPAPSAPA |
| | ASPAAPAPASPAAPAPSAPAASPAAPAPASPAAPAPSAPAASP |
| | AAPAPASPAAPAPSAPAASPAAPAPASPAAPAPSAPAASPAAP |
| | APASPAAPAPSAPAASPAAPAPASPAAPAPSAPAAHHHHHH |
| SIGNAL peptide-AHF-hLPL(28- | METDTLLLWVLLLWVPGSTGGLNDIFEAQKIEWHEGGHHHH |
| 475)-(G4S)4-hGPIHBP1(21- | HHDYKDDDDKADQRRDFIDIESKFALRTPEDTAEDTCHLIPG |
| 151)-(G4S)3-PAS600 | VAESVATCHFNHSSKTFMVIHGWTVTGMYESWVPKLVAALY |
| (SEQ ID NO: 36) | KREPDSNVIVVDWLSRAQEHYPVSAGYTKLVGQDVARFINW |
| | MEEEFNYPLDNVHLLGYSLGAHAAGIAGSLTNKKVNRITGLDP |
| | AGPNFEYAEAPSRLSPDDADFVDVLHTFTRGSPGRSIGIQKPV |
| | GHVDIYPNGGTFQPGCNIGEAIRVIAERGLGDVDQLVKCSHE |
| | RSIHLFIDSLLNEENPSKAYRCSSKEAFEKGLCLSCRKNRCNN |
| | LGYEINKVRAKASSKMYLKTRSQMPYKVFHYQVKIHFSGTES |
| | ETHTNQAFEISLYGTVAESENIPFTLPEVSTNKTYSFLIYTEVDI |
| | GELLMLKLKWKSDSYFSWSDWWSSPGFAIQKIRVKAGETQK |
| | KVIFCSREKVSHLQKGKAPAVFVKCHDKSLNKKSGGGGGSG |
| | GGGSGGGGSGGGGSQTQQEEEEEDEDHGPDDYDEEDEDE |
| | VEEEETNRLPGGRSRVLLRCYTCKSLPRDERCNLTQNCSHGQ |
| | TCTTLIAHGNTESGLLTTHSTWCTDSCQPITKTVEGTQVTMT |
| | CCQSSLCNVPPWQSSRVQDPTGGGGGSGGGGSGGGGSSS |
| | AAASSSASPAAPAPASPAAPAPSAPAASPAAPAPASPAAPAPS |
| | APAASPAAPAPASPAAPAPSAPAASPAAPAPASPAAPAPSAPA |
| | ASPAAPAPASPAAPAPSAPAASPAAPAPASPAAPAPSAPAASP |
| | AAPAPASPAAPAPSAPAASPAAPAPASPAAPAPSAPAASPAAP |
| | APASPAAPAPSAPAASPAAPAPASPAAPAPSAPAASPAAPAPA |
| | SPAAPAPSAPAASPAAPAPASPAAPAPSAPAASPAAPAPASPA |
| | APAPSAPAASPAAPAPASPAAPAPSAPAASPAAPAPASPAAPA |
| | PSAPAASPAAPAPASPAAPAPSAPAASPAAPAPASPAAPAPSA |
| | PAASPAAPAPASPAAPAPSAPAASPAAPAPASPAAPAPSAPAA |
| | SPAAPAPASPAAPAPSAPAASPAAPAPASPAAPAPSAPAASPA |
| | APAPASPAAPAPSAPAASPAAPAPASPAAPAPSAPAASPAAPA |
| | PASPAAPAPSAPAASPAAPAPASPAAPAPSAPAASPAAPAPAS |
| | PAAPAPSAPAASPAAPAPASPAAPAPSAPAASPAAPAPASPAA |
| | PAPSAPAASPAAPAPASPAAPAPSAPAASPAAPAPASPAAPAP |
| | SAPAAHHHHHH* |
| SIGNAL peptide-AHF-hLPL(28- | METDTLLLWVLLLWVPGSTGGLNDIFEAQKIEWHEGGHHHH |
| 475)-(G4S)4-hGPIHBP1(21- | HHDYKDDDDKADQRRDFIDIESKFALRTPEDTAEDTCHLIPG |
| 151)-(G4S)3-CA645scFv-LV- | VAESVATCHFNHSSKTFMVIHGWTVTGMYESWVPKLVAALY |
| HV | KREPDSNVIVVDWLSRAQEHYPVSAGYTKLVGQDVARFINW |
| (SEQ ID NO: 37) | MEEEFNYPLDNVHLLGYSLGAHAAGIAGSLTNKKVNRITGLDP |
| | AGPNFEYAEAPSRLSPDDADFVDVLHTFTRGSPGRSIGIQKPV |
| | GHVDIYPNGGTFQPGCNIGEAIRVIAERGLGDVDQLVKCSHE |
| | RSIHLFIDSLLNEENPSKAYRCSSKEAFEKGLCLSCRKNRCNN |
| | LGYEINKVRAKASSKMYLKTRSQMPYKVFHYQVKIHFSGTES |
| | ETHTNQAFEISLYGTVAESENIPFTLPEVSTNKTYSFLIYTEVDI |
| | GELLMLKLKWKSDSYFSWSDWWSSPGFAIQKIRVKAGETQK |
| | KVIFCSREKVSHLQKGKAPAVFVKCHDKSLNKKSGGGGGSG |
| | GGGSGGGGSGGGGSQTQQEEEEEDEDHGPDDYDEEDEDE |
| | VEEEETNRLPGGRSRVLLRCYTCKSLPRDERCNLTQNCSHGQ |
| | TCTTLIAHGNTESGLLTTHSTWCTDSCQPITKTVEGTQVTMT |
| | CCQSSLCNVPPWQSSRVQDPTGGGGGSGGGGSGGGGSDI |
| | QMTQSPSSVSASVGDRVTITCQSSPSVWSNFLSWYQQKPG |
| | KAPKLLIYEASKLTSGVPSRFSGSGSGTDFTLTISSLQPEDFAT |
| | YYCGGGYSSISDTTFGGGTKVEIKGGGGSGGGGSGGGGSG |
| | GGGSEVQLLESGGGLVQPGGSLRLSCAVSGIDLSNYAINWV |
| | RQAPGKGLEWIGIIWASGTTFYATWAKGRFTISRDNSKNTVY |
| | LQMNSLRAEDTAVYYCARTVPGYSTAPYFDLWGQGTLVTVSS |
| SIGNAL peptide-CA645Fab_HC | METDTLLLWVLLLWVPGSTGEVQLLESGGGLVQPGGSLRLSC |
| (cotransf. CA645Fab_LC)- | AVSGIDLSNYAINWVRQAPGKGLEWIGIIWASGTTFYATWAK |
| (G45)3-hLPL(28-475)-(G4S)4- | GRFTISRDNSKNTVYLQMNSLRAEDTAVYYCARTVPGYSTAP |
| hGPIHBP1(21-151)-FHA (SEQ | YFDLWGQGTLVTVSSASTKGPSVFPLAPSSKSTSGGTAALGC |
| ID NO: 38) | LVKDYFPEPVTVSWNSGALTSGVHTFPAVLQSSGLYSLSSVV |
| | TVPSSSLGTQTYICNVNHKPSNTKVDKKVEPKSCGGGGSAD |
| | QRRDFIDIESKFALRTPEDTAEDTCHLIPGVAESVATCHFNHS |
| | SKTFMVIHGWTVTGMYESWVPKLVAALYKREPDSNVIVVDW |
| | LSRAQEHYPVSAGYTKLVGQDVARFINWMEEEFNYPLDNVHL |
| | LGYSLGAHAAGIAGSLTNKKVNRITGLDPAGPNFEYAEAPSRL |
| | SPDDADFVDVLHTFTRGSPGRSIGIQKPVGHVDIYPNGGTFQ |
| | PGCNIGEAIRVIAERGLGDVDQLVKCSHERSIHLFIDSLLNEE |
| | NPSKAYRCSSKEAFEKGLCLSCRKNRCNNLGYEINKVRAKRS |
| | SKMYLKTRSQMPYKVFHYQVKIHFSGTESETHTNQAFEISLYG |
| | TVAESENIPFTLPEVSTNKTYSFLIYTEVDIGELLMLKLKWKSD |
| | SYFSWSDWWSSPGFAIQKIRVKAGETQKKVIFCSREKVSHL |
| | QKGKAPAVFVKCHDKSLNKKSGGGGGSGGGGSGGGGSGG |
| | GGSQTQQEEEEEDEDHGPDDYDEEDEDEVEEEETNRLPGGR |
| | SRVLLRCYTCKSLPRDERCNLTQNCSHGQTCTTLIAHGNTES |
| | GLLTTHSTWCTDSCQPITKTVEGTQVTMTCCQSSLCNVPPW |
| | QSSRVQDPTGDYKDDDDKHHHHHHGGGLNDIFEAQKIEWH |
| | E |
| SIGNAL peptide- | METDTLLLWVLLLWVPGSTGEVQLLESGGGLVQPGGSLRLSC |
| NOV2704Fab_HC (cotransf. | AASGFTFSDYAMSWVRQAPGKGLEWVSAISYSGSYTYYADS |
| NOV2704Fab_LC)-(G4S)3- | VKGRFTISRDNSKNTLYLQMNSLRAEDTAVYYCARGRYGMDY |
| hLPL(28-475)-(G4S)4- | WGQGTLVTVSSASTKGPSVFPLAPSSKSTSGGTAALGCLVKD |
| hGPIHBP1(21-151)-FHA (SEQ | YFPEPVTVSWNSGALTSGVHTFPAVLQSSGLYSLSSVVTVPS |
| ID NO: 39) | SSLGTQTYICNVNHKPSNTKVDKKVEPKSCGGGGSGGGGSG |
| | GGGSADQRRDFIDIESKFALRTPEDTAEDTCHLIPGVAESVAT |
| | CHFNHSSKTFMVIHGWTVTGMYESWVPKLVAALYKREPDSN |
| | VIVVDWLSRAQEHYPVSAGYTKLVGQDVARFINWMEEEFNYP |
| | LDNVHLLGYSLGAHAAGIAGSLTNKKVNRITGLDPAGPNFEY |
| | AEAPSRLSPDDADFVDVLHTFTRGSPGRSIGIQKPVGHVDIYP |
| | NGGTFQPGCNIGEAIRVIAERGLGDVDQLVKCSHERSIHLFID |
| | SLLNEENPSKAYRCSSKEAFEKGLCLSCRKNRCNNLGYEINKV |
| | RAKRSSKMYLKTRSQMPYKVFHYQVKIHFSGTESETHTNQAF |
| | EISLYGTVAESENIPFTLPEVSTNKTYSFLIYTEVDIGELLMLKL |
| | KWKSDSYFSWSDWWSSPGFAIQKIRVKAGETQKKVIFCSRE |
| | KVSHLQKGKAPAVFVKCHDKSLNKKSGGGGGSGGGGSGGG |
| | GSGGGGSQTQQEEEEEDEDHGPDDYDEEDEDEVEEEETNRL |
| | PGGRSRVLLRCYTCKSLPRDERCNLTQNCSHGQTCTTLIAHG |
| | NTESGLLTTHSTWCTDSCQPITKTVEGTQVTMTCCQSSLCNV |
| | PPWQSSRVQDPTGDYKDDDDKHHHHHHGGGLNDIFEAQKI |
| | EWHE |
| hLPL(28-475)-(GGGGS)₄- | ADQRRDFIDIESKFALRTPEDTAEDTCHLIPGVAESVATCHFN |
| hGPIHBP1(21-151) (SEQ ID | HSSKTFMVIHGWTVTGMYESWVPKLVAALYKREPDSNVIVV |
| NO: 40) | DWLSRAQEHYPVSAGYTKLVGQDVARFINWMEEEFNYPLDN |
| | VHLLGYSLGAHAAGIAGSLTNKKVNRITGLDPAGPNFEYAEAP |
| | SRLSPDDADFVDVLHTFTRGSPGRSIGIQKPVGHVDIYPNGG |
| | TFQPGCNIGEAIRVIAERGLGDVDQLVKCSHERSIHLFIDSLL |
| | NEENPSKAYRCSSKEAFEKGLCLSCRKNRCNNLGYEINKVRA |
| | KRSSKMYLKTRSQMPYKVFHYQVKIHFSGTESETHTNQAFEI |
| | SLYGTVAESENIPFTLPEVSTNKTYSFLIYTEVDIGELLMLKLK |
| | WKSDSYFSWSDWWSSPGFAIQKIRVKAGETQKKVIFCSREK |
| | VSHLQKGKAPAVFVKCHDKSLNKKSGGGGGSGGGGSGGGG |
| | SGGGGSQTQQEEEEEDEDHGPDDYDEEDEDEVEEEETNRLP |
| | GGRSRVLLRCYTCKSLPRDERCNLTQNCSHGQTCTTLIAHGN |
| | TESGLLTTHSTWCTDSCQPITKTVEGTQVTMTCCQSSLCNVP |
| | PWQSSRVQDPTG |
| SIGNAL peptide-AHF-hLPL(28- | METDTLLLWVLLLWVPGSTGGLNDIFEAQKIEWHEGGHHHH |
| 475)R294A-(GGGGS)₄- | HHDYKDDDDKADQRRDFIDIESKFALRTPEDTAEDTCHLIPG |
| hGPIHBP1(21-151) (SEQ ID | VAESVATCHFNHSSKTFMVIHGWTVTGMYESWVPKLVAALY |
| NO: 46) | KREPDSNVIVVDWLSRAQEHYPVSAGYTKLVGQDVARFINW |
| | MEEEFNYPLDNVHLLGYSLGAHAAGIAGSLTNKKVNRITGLDP |
| | AGPNFEYAEAPSRLSPDDADFVDVLHTFTRGSPGRSIGIQKPV |
| | GHVDIYPNGGTFQPGCNIGEAIRVIAERGLGDVDQLVKCSHE |
| | RSIHLFIDSLLNEENPSKAYRCSSKEAFEKGLCLSCRKNRCNN |
| | LGYEINKVAAKRSSKMYLKTRSQMPYKVFHYQVKIHFSGTES |
| | ETHTNQAFEISLYGTVAESENIPFTLPEVSTNKTYSFLIYTEVDI |
| | GELLMLKLKWKSDSYFSWSDWWSSPGFAIQKIRVKAGETQK |
| | KVIFCSREKVSHLQKGKAPAVFVKCHDKSLNKKSGGGGGSG |
| | GGGSGGGGSGGGGSQTQQEEEEEDEDHGPDDYDEEDEDE |
| | VEEEETNRLPGGRSRVLLRCYTCKSLPRDERCNLTQNCSHGQ |
| | TCTTLIAHGNTESGLLTTHSTWCTDSCQPITKTVEGTQVTMT |
| | CCQSSLCNVPPWQSSRVQDPTG |
| AHF-hLPL(28-475)R294A- | GLNDIFEAQKIEWHEGGHHHHHHDYKDDDDKADQRRDFIDI |
| (GGGGS)4-hGPIHBP1(21-151) | ESKFALRTPEDTAEDTCHLIPGVAESVATCHFNHSSKTFMVIH |
| (SEQ ID NO: 47) | GWTVTGMYESWVPKLVAALYKREPDSNVIVVDWLSRAQEHY |
| | PVSAGYTKLVGQDVARFINWMEEEFNYPLDNVHLLGYSLGAH |
| | AAGIAGSLTNKKVNRITGLDPAGPNFEYAEAPSRLSPDDADFV |
| | DVLHTFTRGSPGRSIGIQKPVGHVDIYPNGGTFQPGCNIGEAI |
| | RVIAERGLGDVDQLVKCSHERSIHLFIDSLLNEENPSKAYRCS |
| | SKEAFEKGLCLSCRKNRCNNLGYEINKVAAKRSSKMYLKTRS |
| | QMPYKVFHYQVKIHFSGTESETHTNQAFEISLYGTVAESENIP |
| | FTLPEVSTNKTYSFLIYTEVDIGELLMLKLKWKSDSYFSWSDW |
| | WSSPGFAIQKIRVKAGETQKKVIFCSREKVSHLQKGKAPAVF |
| | VKCHDKSLN KKSGGGGGSGGGGSGGGGSGGGGSQTQQEE |
| | EEEDEDHGPDDYDEEDEDEVEEEETNRLPGGRSRVLLRCYTC |
| | KSLPRDERCNLTQNCSHGQTCTTLIAHGNTESGLLTTHSTWC |
| | TDSCQPITKTVEGTQVTMTCCQSSLCNVPPWQSSRVQDPTG |
| hLPL(28-475)R294A- | ADQRRDFIDIESKFALRTPEDTAEDTCHLIPGVAESVATCHFN |
| (GGGGS)4-hGPIHBP1(21-151) | HSSKTFMVIHGWTVTGMYESWVPKLVAALYKREPDSNVIVV |
| (SEQ ID NO: 48) | DWLSRAQEHYPVSAGYTKLVGQDVARFINWMEEEFNYPLDN |
| | VHLLGYSLGAHAAGIAGSLTNKKVNRITGLDPAGPNFEYAEAP |
| | SRLSPDDADFVDVLHTFTRGSPGRSIGIQKPVGHVDIYPNGG |
| | TFQPGCNIGEAIRVIAERGLGDVDQLVKCSHERSIHLFIDSLL |
| | NEENPSKAYRCSSKEAFEKGLCLSCRKNRCNNLGYEINKVAA |
| | KRSSKMYLKTRSQMPYKVFHYQVKIHFSGTESETHTNQAFEI |
| | SLYGTVAESENIPFTLPEVSTNKTYSFLIYTEVDIGELLMLKLK |
| | WKSDSYFSWSDWWSSPGFAIQKIRVKAGETQKKVIFCSREK |
| | VSHLQKGKAPAVFVKCHDKSLNKKSGGGGGSGGGGSGGGG |
| | SGGGGSQTQQEEEEEDEDHGPDDYDEEDEDEVEEEETNRLP |
| | GGRSRVLLRCYTCKSLPRDERCNLTQNCSHGQTCTTLIAHGN |
| | TESGLLTTHSTWCTDSCQPITKTVEGTQVTMTCCQSSLCNVP |
| | PWQSSRVQDPTG |
| SIGNAL peptide-LPL(28-475)- | METDTLLLWVLLLWVPGSTGADQRRDFIDIESKFALRTPEDTA |
| (G4S)4-hGPIHBP1(21-151)- | EDTCHLIPGVAESVATCHFNHSSKTFMVIHGWTVTGMYESW |
| FLAG-HIS₆-Avi (SEQ ID NO: | VPKLVAALYKREPDSNVIVVDWLSRAQEHYPVSAGYTKLVGQ |
| 51) | DVARFINWMEEEFNYPLDNVHLLGYSLGAHAAGIAGSLTNKK |
| | VNRITGLDPAGPNFEYAEAPSRLSPDDADFVDVLHTFTRGSP |
| | GRSIGIQKPVGHVDIYPNGGTFQPGCNIGEAIRVIAERGLGDV |
| | DQLVKCSHERSIHLFIDSLLNEENPSKAYRCSSKEAFEKGLCL |
| | SCRKNRCNNLGYEINKVRAKRSSKMYLKTRSQMPYKVFHYQ |
| | VKIHFSGTESETHTNQAFEISLYGTVAESENIPFTLPEVSTNKT |
| | YSFLIYTEVDIGELLMLKLKWKSDSYFSWSDWWSSPGFAIQK |
| | IRVKAGETQKKVIFCSREKVSHLQKGKAPAVFVKCHDKSLNK |
| | KSGGGGGSGGGGSGGGGSGGGGSQTQQEEEEEDEDHGPD |
| | DYDEEDEDEVEEEETNRLPGGRSRVLLRCYTCKSLPRDERCN |
| | LTQNCSHGQTCTTLIAHGNTESGLLTTHSTWCTDSCQPITKT |
| | VEGTQVTMTCCQSSLCNVPPWQSSRVQDPTGDYKDDDDKH |
| | HHHHHGGGLNDIFEAQKIEWHE |
| LPL(28-475)-(G4S)4- | ADQRRDFIDIESKFALRTPEDTAEDTCHLIPGVAESVATCHFN |
| hGPIHBP1(21-151)-FLAG-HIS₆- | HSSKTFMVIHGWTVTGMYESWVPKLVAALYKREPDSNVIVV |
| Avi (SEQ ID NO: 53) | DWLSRAQEHYPVSAGYTKLVGQDVARFINWMEEEFNYPLDN |
| | VHLLGYSLGAHAAGIAGSLTNKKVNRITGLDPAGPNFEYAEAP |
| | SRLSPDDADFVDVLHTFTRGSPGRSIGIQKPVGHVDIYPNGG |
| | TFQPGCNIGEAIRVIAERGLGDVDQLVKCSHERSIHLFIDSLL |
| | NEENPSKAYRCSSKEAFEKGLCLSCRKNRCNNLGYEINKVRA |
| | KRSSKMYLKTRSQMPYKVFHYQVKIHFSGTESETHTNQAFEI |
| | SLYGTVAESENIPFTLPEVSTNKTYSFLIYTEVDIGELLMLKLK |
| | WKSDSYFSWSDWWSSPGFAIQKIRVKAGETQKKVIFCSREK |
| | VSHLQKGKAPAVFVKCHDKSLNKKSGGGGGSGGGGSGGGG |
| | SGGGGSQTQQEEEEEDEDHGPDDYDEEDEDEVEEEETNRLP |
| | GGRSRVLLRCYTCKSLPRDERCNLTQNCSHGQTCTTLIAHGN |
| | TESGLLTTHSTWCTDSCQPITKTVEGTQVTMTCCQSSLCNVP |
| | PWQSSRVQDPTGDYKDDDDKHHHHHHGGGLNDIFEAQKIE |
| | WHE |
| LPL(28-475)-(G4S)4- | ADQRRDFIDIESKFALRTPEDTAEDTCHLIPGVAESVATCHFN |
| hGPIHBP1(21-151) (SEQ ID | HSSKTFMVIHGWTVTGMYESWVPKLVAALYKREPDSNVIVV |
| NO: 54) | DWLSRAQEHYPVSAGYTKLVGQDVARFINWMEEEFNYPLDN |
| | VHLLGYSLGAHAAGIAGSLTNKKVNRITGLDPAGPNFEYAEAP |
| | SRLSPDDADFVDVLHTFTRGSPGRSIGIQKPVGHVDIYPNGG |
| | TFQPGCNIGEAIRVIAERGLGDVDQLVKCSHERSIHLFIDSLL |
| | NEENPSKAYRCSSKEAFEKGLCLSCRKNRCNNLGYEINKVRA |
| | KRSSKMYLKTRSQMPYKVFHYQVKIHFSGTESETHTNQAFEI |
| | SLYGTVAESENIPFTLPEVSTNKTYSFLIYTEVDIGELLMLKLK |
| | WKSDSYFSWSDWWSSPGFAIQKIRVKAGETQKKVIFCSREK |
| | VSHLQKGKAPAVFVKCHDKSLNKKSGGGGGSGGGGSGGGG |
| | SGGGGSQTQQEEEEEDEDHGPDDYDEEDEDEVEEEETNRLP |
| | GGRSRVLLRCYTCKSLPRDERCNLTQNCSHGQTCTTLIAHGN |
| | TESGLLTTHSTWCTDSCQPITKTVEGTQVTMTCCQSSLCNVP |
| | PWQSSRVQDPTG |

### Nucleic Acid Molecules Encoding Fusion Polypeptides of the Disclosure

Another aspect of the disclosure pertains to nucleic acid molecules that encode a fusion polypeptide of the disclosure. Such nucleic acid molecules may be DNA or RNA. Unless specifically limited herein, the term encompasses nucleic acids containing known analogues of natural nucleotides that have similar properties as the reference nucleic acid and are metabolized in a manner similar to naturally occurring nucleotides. Examples of such analogs include, without limitation, phosphorothioates, phosphoramidates, methyl phosphonates, chiral-methyl phosphorates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994, the contents of each of which are incorporated herein for this purpose).

Thus the disclosure also provides a nucleic acid comprising a nucleotide sequence encoding the polypeptide sequence of any one or more of SEQ ID NOs: 33-40, 46-48, 51, 53, 54, or 55. Thus the disclosure also provides a nucleic acid comprising a nucleotide sequence encoding any one of the polypeptide sequences in Table 1, optionally without a signal peptide and/or optionally without a tag or marker.

The disclosure further provides a nucleic acid comprising a nucleotide sequence having at least about 80%, at least about 85%, at least about 90%, at least about 95%, at least about 97%, or at least about 99% sequence identity with a nucleic acid encoding any one of SEQ ID NOS: 33-40, 46-48, 51, 53, 54, or 55. The disclosure further provides a nucleic acid comprising a nucleotide sequence having at least about 80%, at least about 85%, at least about 90%, at least about 95%, at least about 97%, or at least about 99% sequence identity with a nucleic acid encoding any one of the amino acid sequences in Table 1, optionally without a signal peptide and/or optionally without a tag or marker. Sequence identity is typically measured along the full length of the reference sequence.

The disclosure further provides a nucleic acid comprising a nucleotide sequence having at least about 80%, at least about 85%, at least about 90%, at least about 95%, at least about 97%, or at least about 99% sequence identity with SEQ ID NO: 44.

The disclosure also provides a nucleic acid comprising the nucleotide sequence of SEQ ID NO: 44. The disclosure also provides a nucleic acid consisting of the nucleotide sequence of SEQ ID NO: 44.

The polynucleotide sequences can be produced by de novo solid-phase DNA synthesis or by PCR mutagenesis of an existing sequence (e.g., sequences as described in the Examples below). Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68: 109, 1979; the diethylphosphoramidite method of Beaucage et al., Tetra. Lett., 22: 1859, 1981; and the solid support method of U.S. Pat. No. 4,458,066, the contents of each of which are incorporated herein for this purpose. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991, the contents of each of which are incorporated herein for this purpose.

### Vectors

The present disclosure also provides vectors comprising one or more nucleic acid molecules of the disclosure.

For expression in host cells, the nucleic acid encoding a fusion polypeptide can be present in a suitable vector and after introduction into a suitable host, the sequence can be expressed to produce the encoded fusion polypeptide according to standard cloning and expression techniques, which are known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989, the contents of each of which are incorporated herein for this purpose). The disclosure also relates to such vectors comprising a nucleic acid sequence according to the disclosure.

Various expression vectors can be employed to express the polynucleotides encoding the fusion polypeptides of the disclosure. Both viral-based and nonviral expression vectors can be used to produce the fusion polypeptides in a host cell, such as a mammalian host cell. Nonviral vectors and systems include plasmids, episomal vectors, typically with an expression cassette for expressing a protein or RNA, and human artificial chromosomes (see, e.g., Harrington et al., Nat Genet. 15:345, 1997, the contents of which are incorporated herein for this purpose). For example, nonviral vectors useful for expression of the polynucleotides and polypeptides of the fusion polypeptides of the disclosure in mammalian (e.g., human) cells include pThioHis A, B and C, pcDNA3.1/His, pEBVHis A, B and C, (Invitrogen, San Diego, Calif.), MPS V vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68: 143, 1992, the contents of each of which are incorporated herein for this purpose.

The choice of expression vector depends on the intended host cells in which the vector is to be expressed. Expression vectors for mammalian host cells can include expression control sequences, such as an origin of replication, a promoter, and an enhancer (see, e.g., Queen, et al., Immunol. Rev. 89:49-68, 1986, the contents of which are incorporated herein for this purpose), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPS V promoter, the tetracycline-inducible CMV promoter (such as the human immediate-early CMV promoter), the constitutive CMV promoter, and promoter-enhancer combinations known in the art.

Cultures of transformed organisms can be expanded under non-inducing conditions without biasing the population for coding sequences whose expression products are better tolerated by the host cells. In addition to promoters, other regulatory elements may also be required or desired for efficient expression of the antibody of the disclosure or fragments thereof. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20: 125, 1994; and Bittner et al., Meth. Enzymol., 153:516, 1987, the contents of each of which are incorporated herein for this purpose). For example, the SV40 enhancer or CMV enhancer may be used to increase expression in mammalian host cells.

Accordingly, the disclosure provides a cloning or expression vector comprising the nucleic acid sequence of SEQ ID NO: 44. The disclosure also provides a cloning or expression vector comprising a nucleic acid comprising a nucleotide sequence having at least about 80%, at least about 85%, at least about 90%, at least about 95%, at least about 96%, at least about 97%, at least about 98%, or at least about 99% sequence identity with a nucleic acid encoding any one of SEQ ID NOS: 33-40, 46-48, 51, 53, 54, or 55. The disclosure also provides a cloning or expression vector comprising a nucleic acid comprising a nucleotide sequence having at least about 80%, at least about 85%, at least about 90%, at least about 95%, at least about 96%, at least about 97%, at least about 98%, or at least about 99% sequence identity with a nucleic acid encoding any one of the amino acid sequences in Table 1, optionally without a signal peptide. Furthermore, the disclosure provides a cloning or expression vector comprising a nucleic acid encoding one or more of SEQ ID NOs: 33-40, 46-48, 51, 53 or 54. Furthermore, the disclosure provides a cloning or expression vector comprising a nucleic acid encoding one or more of the amino acid sequences in Table 1, optionally without a signal peptide.

### Host Cells

Recombinant cells including prokaryotic (e.g., E. coli) or eukaryotic cells (e.g., insect cells, yeast cells, or mammalian cells) comprising a nucleic acid of the disclosure, vector of the disclosure, or combinations of either or both thereof are provided. Thus cells, such as yeast, bacterial (e.g., E. coli), and mammalian cells (e.g., immortalized mammalian cells) comprising a nucleic acid of the disclosure, vector of the disclosure, or combinations of either or both thereof are provided. Such cells are generally utilized for the expression of the fusion polypeptides according to the disclosure. The nucleic acid or vector may be transfected into a host cell by standard techniques.

The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is possible to express the fusion polypeptides of the disclosure in either prokaryotic or eukaryotic host cells. Representative host cells include many E. coli strains, mammalian cell lines, such as CHO, CHO-K1, and HEK293; insect cells, such as Sf9 cells; and yeast cells, such as S. cerevisiae and P. pastoris.

Mammalian host cells for expressing the fusion polypeptides of the disclosure may include Chinese Hamster Ovary (CHO cells) (including dhfr− CHO cells, described by Urlaub and Chasin, 1980 Proc. Natl. Acad. Sci. USA 77:4216-4220 and used with a DH FR selectable marker, e.g., as described in R. J. Kaufman and P. A. Sharp, 1982 Mol. Biol. 159:601-621; the contents of each of which are incorporated herein for this purpose), NSO myeloma cells, COS cells and SP2 cells. In one embodiment, the host cells are CHO K1PD cells. In another embodiment the host cells are NSO1 cells. In particular, for use with NSO myeloma cells, another expression system is the GS gene expression system shown in WO 87/04462, WO 89/01036 and EP 338,841, the contents of each of which are incorporated herein for this purpose. When recombinant expression vectors encoding fusion polypeptides are introduced into mammalian host cells, the fusion polypeptides may be produced by culturing the host cells for a period of time sufficient to allow for expression of the fusion polypeptide in the host cells or secretion of the fusion polypeptide into the culture medium in which the host cells are grown. Fusion polypeptides can be recovered from the culture medium using standard protein purification methods.

### Pharmaceutical Compositions

The disclosure also provides pharmaceutical compositions comprising a fusion polypeptide, nucleic acid, vector or host cell as described herein. Such pharmaceutical compositions can comprise a therapeutically effective amount of the fusion polypeptide, nucleic acid, vector or host cell and a pharmaceutically or physiologically acceptable diluent and/or carrier. The carrier is generally selected to be suitable for the intended mode of administration and can include agents for modifying, maintaining, or preserving, for example, the pH, osmolarity, viscosity, clarity, color, isotonicity, odor, sterility, stability, rate of dissolution or release, adsorption, and/or penetration of the composition. Typically, these carriers include aqueous or alcoholic/aqueous solutions, emulsions or suspensions, including saline and/or buffered media.

Suitable agents for inclusion in the pharmaceutical compositions may include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, diluents, excipients and/or pharmaceutical adjuvants.

Parenteral vehicles may include, but are not limited to, sodium chloride solution, Ringer's dextrose, dextrose and sodium chloride and lactated Ringer's. Suitable physiologically-acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some cases one might include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents and inert gases, may also be present. The precise formulation will depend on the route of administration. Additional relevant principle, methods and components for pharmaceutical formulations are well known (see, e.g., Allen, Loyd V. Ed, (2012) Remington's Pharmaceutical Sciences, 22^{nd} Edition, the contents of which are incorporated herein for this purpose).

A pharmaceutical composition of the present disclosure can be administered by one or more routes of administration using one or more of a variety of methods known in the art. As will be appreciated by the skilled artisan, the route and/or mode of administration will vary depending upon the desired results. Routes of administration for fusion polypeptides of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

Alternatively, the fusion polypeptide described herein can be administered by a non-parenteral route, such as a topical, epidermal or mucosal route of administration, for example, intranasally, orally, vaginally, rectally, sublingually, or topically.

In certain embodiments, the pharmaceutical composition is for subcutaneous administration. Suitable formulation components and methods for subcutaneous administration of polypeptide therapeutics (e.g., antibodies, fusion polypeptides and the like) are known in the art, see, for example, US2011/0044977, U.S. Pat. Nos. 8,465,739 and 8,476,239, the contents of each of which are incorporated herein for this purpose.

Typically, the pharmaceutical compositions for subcutaneous administration contain suitable stabilizers (e.g., amino acids, such as methionine, and/or saccharides such as sucrose), buffering agents, and/or tonicifying agents.

### Uses and Methods

The fusion polypeptides described herein have therapeutic utility. For example, these fusion polypeptides can be administered to a subject and may be used in the treatment of disease, prophylaxis and/or for delaying the onset of disease symptoms.

Thus, the disclosure provides a fusion polypeptide, nucleic acid, vector, host cell, or pharmaceutical composition of the disclosure for use in therapy or for use as a medicament for the treatment of a disease or disorder. The disclosure further provides a fusion polypeptide, nucleic acid, vector, host cell, or pharmaceutical composition of the disclosure for use in the treatment of a pathological disorder. The disclosure also provides the use of a fusion polypeptide, nucleic acid, vector, host cell, or pharmaceutical composition of the disclosure in the manufacture of a medicament for the treatment of a pathological disorder. The disclosure further provides a method of treating a patient or subject suffering from a pathological disorder comprising administering a therapeutically effective amount of a fusion polypeptide, nucleic acid, vector, host cell or pharmaceutical composition of the disclosure to said patient or subject.

In one embodiment the patient or subject being treated may have a blood triglyceride level of 150 mg/dL or more (for example, 200 mg/dL, 250 mg/dL, 300 mg/dL, 350 mg/dL, 400 mg/dL, 500 mg/dL, 600 mg/dL, 700 mg/dL, 800 mg/dL, 900 mg/dL, 1000 mg/dL, 1100 mg/dL, 1200 mg/dL, 1300 mg/dL, 1400 mg/dL, 1500 mg/dL or more) prior to treatment.

As used herein, the term “pathological disorder” includes, but is not limited to chylomicronemia (including Familial chylomicronemia syndrome, polygenic late-onset chylomicronemia and early-onset chylomicronemia), hyperlipidemic pancreatitis, hypertriglyceridemia, abdominal pain, recurrent acute pancreatitis, eruptive cutaneous xanthomata, lipemia retinalis, hepatosplenomegaly, diabetes, obesity, cardiovascular disease, chronic kidney disease, non-alcoholic fatty liver disease, hypertriglyceridemic pancreatitis, hepatosteatosis, metabolic syndrome, ischemic heart disease, and microvascular pathology.

Thus, in one embodiment, the disclosure provides a fusion polypeptide, nucleic acid, vector, host cell, or pharmaceutical composition of the disclosure for use in the treatment of chylomicronemia (e.g., familial chylomicronemia syndrome, polygenic late-onset chylomicronemia and/or early-onset chylomicronemia).

### General

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

The term “about” in relation to a numerical value x means, for example, x±5%.

Features of each aspect of the disclosure may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### EXAMPLES

### General Methods

### Expression Plasmids

Mammalian expression vectors for LPL, GPIHBP1, ANGPTL3 and ANGPTL4 were synthesized and the sequences of the open reading frames are listed in Table 2.

TABLE 2
Amino acid sequences of recombinant human LPL, soluble human
GPIHBP1, human ANGPTL3, human ANGPTL4 and human LPL-GPIHBP1 fusion
polypeptides

| Recombinant protein | Amino Acid Sequence |
|---|---|
| Mature Human LPL (SEQ | ADQRRDFIDIESKFALRTPEDTAEDTCHLIPGVAESVATCHFNH |
| ID NO: 2) | SSKTFMVIHGWTVTGMYESWVPKLVAALYKREPDSNVIVVDWL |
| | SRAQEHYPVSAGYTKLVGQDVARFINWMEEEFNYPLDNVHLLG |
| | YSLGAHAAGIAGSLTNKKVNRITGLDPAGPNFEYAEAPSRLSPD |
| | DADFVDVLHTFTRGSPGRSIGIQKPVGHVDIYPNGGTFQPGCNI |
| | GEAIRVIAERGLGDVDQLVKCSHERSIHLFIDSLLNEENPSKAYR |
| | CSSKEAFEKGLCLSCRKNRCNNLGYEINKVRAKRSSKMYLKTRS |
| | QMPYKVFHYQVKIHFSGTESETHTNQAFEISLYGTVAESENIPFT |
| | LPEVSTNKTYSFLIYTEVDIGELLMLKLKWKSDSYFSWSDWWS |
| | SPGFAIQKIRVKAGETQKKVIFCSREKVSHLQKGKAPAVFVKCH |
| | DKSLNKKSG |
| His6-Human LPL(28-475) | HHHHHHADQRRDFIDIESKFALRTPEDTAEDTCHLIPGVAESVA |
| (SEQ ID NO: 41) | TCHFNHSSKTFMVIHGWTVTGMYESWVPKLVAALYKREPDSNV |
| | IVVDWLSRAQEHYPVSAGYTKLVGQDVARFINWMEEEFNYPLD |
| | NVHLLGYSLGAHAAGIAGSLTNKKVNRITGLDPAGPNFEYAEAP |
| | SRLSPDDADFVDVLHTFTRGSPGRSIGIQKPVGHVDIYPNGGTF |
| | QPGCNIGEAIRVIAERGLGDVDQLVKCSHERSIHLFIDSLLNEEN |
| | PSKAYRCSSKEAFEKGLCLSCRKNRCNNLGYEINKVRAKRSSK |
| | MYLKTRSQMPYKVFHYQVKIHFSGTESETHTNQAFEISLYGTVA |
| | ESENIPFTLPEVSTNKTYSFLIYTEVDIGELLMLKLKWKSDSYFS |
| | WSDWWSSPGFAIQKIRVKAGETQKKVIFCSREKVSHLQKGKA |
| | PAVFVKCHDKSLNKKSG |
| Human LPL(28-475)-FHA | ADQRRDFIDIESKFALRTPEDTAEDTCHLIPGVAESVATCHFNH |
| (SEQ ID NO: 42) | SSKTFMVIHGWTVTGMYESWVPKLVAALYKREPDSNVIVVDWL |
| | SRAQEHYPVSAGYTKLVGQDVARFINWMEEEFNYPLDNVHLLG |
| | YSLGAHAAGIAGSLTNKKVNRITGLDPAGPNFEYAEAPSRLSPD |
| | DADFVDVLHTFTRGSPGRSIGIQKPVGHVDIYPNGGTFQPGCNI |
| | GEAIRVIAERGLGDVDQLVKCSHERSIHLFIDSLLNEENPSKAYR |
| | CSSKEAFEKGLCLSCRKNRCNNLGYEINKVRAKRSSKMYLKTRS |
| | QMPYKVFHYQVKIHFSGTESETHTNQAFEISLYGTVAESENIPFT |
| | LPEVSTNKTYSFLIYTEVDIGELLMLKLKWKSDSYFSWSDWWS |
| | SPGFAIQKIRVKAGETQKKVIFCSREKVSHLQKGKAPAVFVKCH |
| | DKSLNKKSGDYKDDDDKHHHHHHGGGLNDIFEAQKIEWHE |
| Human soluble GPIHBP1 | QTQQEEEEEDEDHGPDDYDEEDEDEVEEEETNRLPGGRSRVLL |
| (aa 21-151) (SEQ ID NO: | RCYTCKSLPRDERCNLTQNCSHGQTCTTLIAHGNTESGLLTTHS |
| 7) | TWCTDSCQPITKTVEGTQVTMTCCQSSLCNVPPWQSSRVQDP |
| | TG |
| Human soluble | QTQQEEEEEDEDHGPDDYDEEDEDEVEEEETNRLPGGRSRVLL |
| GPIHBP1(21-151)- FLAG- | RCYTCKSLPRDERCNLTQNCSHGQTCTTLIAHGNTESGLLTTHS |
| HIS₆-Avi (SEQ ID NO: 49) | TWCTDSCQPITKTVEGTQVTMTCCQSSLCNVPPWQSSRVQDP |
| | TGDYKDDDDKHHHHHHGGGLNDIFEAQKIEWHE |
| Human ANGPTL4(26-406)- | GPVQSKSPRFASWDEMNVLAHGLLQLGQGLREHAERTRSQLS |
| FLAG-6HIS-Avi (SEQ ID | ALERRLSACGSACQGTEGSTDLPLAPESRVDPEVLHSLQTQLKA |
| NO: 43) | QNSRIQQLFHKVAQQQRHLEKQHLRIQHLQSQFGLLDHKHLDH |
| | EVAKPARRKRLPEMAQPVDPAHNVSRLHRLPRDCQELFQVGER |
| | QSGLFEIQPQGSPPFLVNCKMTSDGGWTVIQRRHDGSVDFNRP |
| | WEAYKAGFGDPHGEFWLGLEKVHSITGDRNSRLAVQLRDWDG |
| | NAELLQFSVHLGGEDTAYSLQLTAPVAGQLGATTVPPSGLSVPF |
| | STWDQDHDLRRDKNCAKSLSGGWWFGTCSHSNLNGQYFRSI |
| | PQQRQKLKKGIFWKTWRGRYYPLQATTMLIQPMAAEAASDYKD |
| | DDDKHHHHHHGGGLNDIFEAQKIEWHE |
| Human ANGPTL3(17-460)- | SRIDQDNSSFDSLSPEPKSRFAMLDDVKILANGLLQLGHGLKDF |
| FLAG-HIS₆-Avi (SEQ ID | VHKTKGQINDIFQKLNIFDQSFYDLSLQTSEIKEEEKELRRTTYK |
| NO: 50) | LQVKNEEVKNMSLELNSKLESLLEEKILLQQKVKYLEEQLTNLIQ |
| | NQPETPEHPEVTSLKTFVEKQDNSIKDLLQTVEDQYKQLNQQH |
| | SQIKEIENQLRRTSIQEPTEISLSSKPRAPRTTPFLQLNEIRNVKH |
| | DGIPAECTTIYNRGEHTSGMYAIRPSNSQVFHVYCDVISGSPWT |
| | LIQHRIDGSQNFNETWENYKYGFGRLDGEFWLGLEKIYSIVKQS |
| | NYVLRIELEDWKDNKHYIEYSFYLGNHETNYTLHLVAITGNVPN |
| | AIPENKDLVFSTWDHKAKGHFNCPEGYSGGWWWHDECGENN |
| | LNGKYNKPRAKSKPERRRGLSWKSQNGRLYSIKSTKMLIHPTD |
| | SESFEDYKDDDDKHHHHHHGGGLNDIFEADKIEWHE |
| LPL(28-475)-(G4S)4- | METDTLLLWVLLLWVPGSTGADQRRDFIDIESKFALRTPEDTAE |
| hGPIHBP1(21-151)-FLAG- | DTCHLIPGVAESVATCHFNHSSKTFMVIHGWTVTGMYESWVPK |
| HIS₆-Avi (SEQ ID NO: 51) | LVAALYKREPDSNVIVVDWLSRAQEHYPVSAGYTKLVGQDVAR |
| | FINWMEEEFNYPLDNVHLLGYSLGAHAAGIAGSLTNKKVNRITG |
| | LDPAGPNFEYAEAPSRLSPDDADFVDVLHTFTRGSPGRSIGIQK |
| | PVGHVDIYPNGGTFQPGCNIGEAIRVIAERGLGDVDQLVKCSHE |
| | RSIHLFIDSLLNEENPSKAYRCSSKEAFEKGLCLSCRKNRCNNL |
| | GYEINKVRAKRSSKMYLKTRSQMPYKVFHYQVKIHFSGTESETH |
| | TNQAFEISLYGTVAESENIPFTLPEVSTNKTYSFLIYTEVDIGELL |
| | MLKLKWKSDSYFSWSDWWSSPGFAIQKIRVKAGETQKKVIFC |
| | SREKVSHLQKGKAPAVFVKCHDKSLNKKSGGGGGSGGGGSG |
| | GGGSGGGGSQTQQEEEEEDEDHGPDDYDEEDEDEVEEEETNR |
| | LPGGRSRVLLRCYTCKSLPRDERCNLTQNCSHGQTCTTLIAHGN |
| | TESGLLTTHSTWCTDSCQPITKTVEGTQVTMTCCQSSLCNVPP |
| | WQSSRVQDPTGDYKDDDDKHHHHHHGGGLNDIFEAQKIEWH |
| | E |

### Enzymes and Reagents

Amplex Red, Resorufin butyrate, Enzchek and DGGR substrates were purchased from Life Technologies. Human VLDL and chylomicrons were from EMD Millipore and Athens research, respectively. BSA was obtained from Sigma. The HR Series NEFA-HR(2) Color Reagent A and HR Series NEFA-HR(2) Color Reagent B were purchased from Wako Diagnostics. Detergents were obtained from Sigma.

### Expression and Purification of Recombinant Proteins

Human LPL: Recombinant human LPL was prepared using the following procedure. HEK293T cells cultured in FreeStyle 293 expression medium were transfected with a mammalian expression plasmid encoding full-length human LPL polypeptide (matching NCBI sequence NM 000237.2) using a standard polyethyleneimine transfection method. At 24 hours after transfection, heparin was added to the culture medium to a final concentration of 3 U/mL, to enhance release of secreted LPL from the cell surface. At 60 hours post-transfection, the culture medium was collected, filtered using a 0.2 μm filter, and glycerol was added to a final concentration of 10% (v/v). The resulting solution was loaded onto a 5 mL Heparin Sepharose HiTrap column which had been pre-equilibrated with Buffer A (50 mM Tris-HCl, 200 mM NaCl, containing 10% (v/v) glycerol, pH 7.2). The column was washed with Buffer A, and LPL was then eluted with step gradients of 500 mM NaCl, 1M NaCl, and 2M NaCl in Buffer A. Elution fractions were assayed for LPL enzyme activity, and protein purity was assessed by SDS-PAGE. The most catalytically active and highest purity LPL eluted with 1 M NaCl. Aliquots of purified human LPL were flash-frozen and stored at −80° C. until use.

Soluble human GPIHBP1: Plasmid containing soluble domain of GPIHBP1 (solGPIHBP1) protein open reading frame with C-terminal end FHA tag was transiently transfected into HEK293T cells using standard polyethylenimine (PEI) transfection methods. Cells were propagated in suspension culture in Freestyle 293 expression media and transfection was carried out at 1×10⁶ cells/mL final cell concentration in 1 liter media. Cells were then harvested by centrifugation, followed by filtration with a 0.22 μm sterile filter. The clarified supernatant was concentrated and buffer exchanged into 50 mM Tris.HCl pH 8, 150 mM NaCl, 10% Glycerol, 20 mM Imidazole using tangential flow filtration (TFF). The concentrated sample was passed over a 5 mL Ni-NTA affinity column equilibrated with buffer containing 50 mM Tris.HCl pH 8, 150 mM NaCl, 10% Glycerol and 20 mM Imidazole. After loading the sample, the column was washed with the same buffer until baseline absorbance at 280 nm was reached. The bound ANGPTL4 protein was then eluted by running a gradient of Imidazole (20 mM to 500 mM). Relevant fractions were pooled, concentrated using Amicon concentrator (Molecular weight cut off 10,000 Da), buffer exchanged using PD-10 columns into storage buffer (PBS), aliquoted and flash frozen in liquid nitrogen.

Human LPL-soluble GPIHBP1 complex: Plasmids encoding human LPL (untagged or with either His or FHA purification tags at the N- or C-terminal end; hLPL), soluble human GPIHBP1 (solGPIHBP1; untagged or with FHA purification tags at C-terminal end), and human LMF1 (hLMF1, Accession no: Q96S06) were transiently transfected into suspension-adapted HEK293T cells using standard polyethylenimine (PEI) transfection method in molar ratio of 3:1:1. The cells were maintained at 37° C. and 5% CO₂ in a shaking incubator for 72 hours. The cells were then harvested by centrifugation and the supernatant was filtered through a 0.22 μm sterile filter. The clarified supernatant was concentrated and buffer exchanged into 20 mM Tris-HCl (pH 7.5), containing 500 mM NaCl, 10% (v/v) glycerol, and 20 mM imidazole using tangential flow filtration (TFF). The concentrated sample was then applied to a Ni-NTA affinity column equilibrated with 20 mM Tris-HCl (pH 7.5), containing 500 mM NaCl, 10% (v/v) glycerol and 20 mM imidazole, and the column was washed with the same buffer until baseline absorbance at 280 nm was reached. The bound hLPL-solGPIHBP1 complex was then eluted by running a gradient of imidazole (20 mM to 500 mM imidazole in 20 mM Tris-HCl, pH 7.5, containing 500 mM NaCl and 10% (v/v) glycerol) and hLPL-solGPIHBP1 complex containing fractions (identified by SDS-PAGE) were pooled, concentrated (Amicon concentrator, Molecular weight cut off 30 kDa) and loaded onto a Superdex 200, 16/60 sizing column equilibrated with running buffer (10 mM Tris, pH 7.5, containing 300 mM NaCl). Peak fractions were analyzed by SDS PAGE, and fractions containing LPL-solGPIHBP1 complex were pooled, concentrated, aliquoted, flash-frozen in liquid nitrogen, and stored at −80° C.

Human LPL-soluble GPIHBP1 fusion polypeptides: Plasmids encoding human LPL-(GGGGS)₄ linker-human GPIHBP1 (FHA purification tags at the N- or C-terminal end), and human LMF1 (hLMF1) were transiently transfected into suspension-adapted HEK293T cells using a standard polyethylenimine (PEI) transfection method in molar ratio of 3:1. The cells were maintained at 37° C. and 5% CO₂ in a shaking incubator for 72 hours. The cells were then harvested by centrifugation and the supernatant was filtered through a 0.22 μm sterile filter. The clarified supernatant was concentrated and buffer exchanged into 50 mM HEPES (pH 7.3), containing 300 mM NaCl, 10% (v/v) glycerol and 30 mM imidazole (Buffer A) using tangential flow filtration (TFF). The concentrated sample was then applied to a HiTrap Ni affinity column (GE) equilibrated with Buffer A and the column was washed with the same buffer until baseline absorbance at 280 nm was reached. The bound fusion polypeptide was then eluted by running a gradient of imidazole (30 mM to 300 mM imidazole in Buffer A and fractions containing fusion polypeptide (identified by SDS-PAGE) were pooled, concentrated (Amicon concentrator, Molecular weight cut off 30 kDa) and loaded onto a Superdex 200, 16/60 sizing column (GE) equilibrated with 10 mM Tris, pH 7.5, containing 300 mM NaCl and 10% glycerol. Peak fractions were analyzed by SDS PAGE, and fractions containing LPL-GPIHBP1 fusion polypeptide were pooled, concentrated, aliquoted, flash-frozen in liquid nitrogen, and stored at −80° C.

### Site-Specific Biotinylation of Proteins

Purified proteins with AviTag were biotinylated as follows: purified protein in 50 mM Bicine pH 8.3 buffer at a final concentration of approximately 1 mg/mL was incubated in the presence of 10 mM ATP, 10 mM magnesium acetate, 0.1 mM biotin, and BirA biotin ligase (Avidity) at 30° C. for 1 hr and then placed at 4° C. overnight. The protein was then concentrated using Amicon concentrator (Molecular weight cut off 10,000), buffered exchanged using PD-10 columns into storage buffer (50 mM Tris.HCl pH 7.4, 150 mM NaCl, 15% Glycerol), aliquoted and flash frozen in liquid nitrogen.

### Biochemical Assays

LPL Enzymatic Activity with Surrogate Substrates EnzChek and Resorufin Butyrate.

### EnzChek Assay

The assays were performed in a 384-well Costar® black plate. To determine LPL activity, varying amounts of LPL protein (either LPL alone, LPL co-purified with GPIHBP1, or LPL-GPIHBP1 fusion) were incubated with 1 μM EnzChek® substrate in assay buffer containing: 20 mM Tris.Cl pH 8.0. 150 mM NaCl, 1.5% BSA, 0.05% Zwittergent®. To determine the ability of ANGPTL4 or ANGPTL3 to inhibit LPL activity, a fixed amount of LPL was pre-incubated with ANGPTL4 or 3 for 5 min followed by the addition of Enzchek substrate. Activity was monitored using Envision multiwell plate reader using excitation and emission wavelengths of 482 and 520 nm, respectively (Perkin Elmer). The rate of hydrolysis was calculated over the initial linear phase of the reaction. Data analysis was performed using Microsoft Excel and GraphPad Prism software.

### Resorufin Butyrate Assay

The assays were performed similar to the Enzchek with the following exceptions that the substrate concentration was 9 μM and the detergent used was 0.025% Zwittergent. The excitation and emission wavelengths were as follows: Ex.: 500 nm; Em.: 593 nm.

LPL Enzymatic Activity with VLDL and CM as a Substrate

The following protocol was used to assess activity of purified LPL. LPL protein (either LPL alone, LPL co-purified with GPIHBP1, or LPL-GPIHBP1 fusion; 20 μL per well, serially diluted 2-fold using assay buffer) was mixed with human VLDL or CM (20 uL per well, diluted using assay buffer) in a 384-well Costar black well plate. To this mix, Amplex Red mix containing the coupled enzyme system (HR series NEFA-HR(2), Wako Diagnostics USA) (20 μL per well, diluted in PBS) was added, and fluorescence of resorufin was monitored continuously for 30 minutes using an Envision multiwell plate reader using excitation and emission wavelengths of 531 and 590 nm, respectively (Perkin Elmer). When stated, a fixed concentration of LPL was pre-incubated with ANGPTL3 or 4 (serially diluted 2-fold using assay buffer) in a volume of 20 μL for 10 min before addition of VLDL. Final assay concentrations were: varying LPL, varying ANGPTL3 or 4, 2.3 μg/mL human VLDL or 10 μg/ml human CM, 0.75 mM ATP, 90 μM coenzyme A, 0.5 U/mL ACO, 1.25 U/mL ACS, 1.2 U/mL HRP, and 10 μM Amplex Red. Data analysis was performed using Microsoft Excel and GraphPad Prism software.

### ELISA Assay for Detection of LPL/GPIHBP1 Complex Disruption by ANGPTL4.

LPL-GPIHBP1 complex or LPL-GPIHBP1 fusion (10 nM, site-specifically biotinylated at the C-terminal end of GPIHBP1) were incubated with increasing concentrations of ANGPTL4 (a 12 point 2-fold serial dilution with highest concentration of 100 nM). The reaction mix was then applied onto a streptavidin coated MSD. The presence of LPL and ANGPTL4 was detected using an LPL or ANGPTL4 specific Abs, which were subsequently quantified using sulpho-tagged secondary Ab. To generate a signal, lx MSD read buffer T was added and the plate was developed using a Sector Imager 6000 (Meso Scale Discovery).

### SPR Assay for Detection of LPL/GPIHBP1 Complex Disruption by ANGPTL4.

LPL-GPIHBP1 complex or fusion with a C-terminal end biotin label was immobilized on a streptavidin surface at a concentration of 1 nM. ANGPTL4 at a concentration of 10 nM was flown over the immobilized complex and the mass was monitored as a function of time.

### TR-FRET Based Assay for Detection ANGPTL4 Binding to the LPL/GPIHBP1 Complex

The assay for TR-FRET based detection of ANGPTL4 binding to the LPL/GPIHBP1 complex was carried out in 384-well plates (ProxiPlate™ 384-well white, Perkin Elmer, USA) in a final volume of 20 μl. The composition of the assay buffer was 20 mM HEPES pH=7.4, 100 mM NaCl, 10% HI-FBS, 5 mM CaCl₂). The assay procedure was as follows: First 4 μL/well of 5x 6HIS-hLPL-HA-Flag/biotinylated hGPIHBP1-Avi complex (50 nM stock in assay buffer, 10 nM complex final concentration) was added to 8 μL/well buffer. Then 4 μL/well 5x non-biotinylated ANGPTL4(26-406)-Flag-6HIS-Avi (final concentrations of 1 to 1000 nM) was added and incubation was carried out at room temperature for 1 hour. After incubation 4 μL 5× anti-HA-Tb/anti-myc-D2 (Cisbio, diluted in assay buffer to 1.25 nM anti-HA-Tb/50 nM streptavidin-D2 stock concentration) (0.3125 nM anti-HA-Tb/10 nM streptavidin-D2 final concentration) was added and the mixture was incubated at room temperature for 3 hours. Finally the plate was read on an Envision plate reader Ex=320 Em=665 Em=615.

### TR-FRET Based Assay for Detection of LPL/ANGPTL4 Complex Disruption

The assay for TR-FRET based detection of LPL/GPIHBP1 complex disruption was carried out in 384-well plates (ProxiPlate™ 384-well white, Perkin Elmer, USA) in a final volume of 20 μl. The composition of the assay buffer was 20 mM HEPES pH=7.4, 100 mM NaCl, 10% HI-FBS, 5 mM CaCl₂). The assay procedure was as follows: First 4 μL/well of 5×6HIS-hLPL-HA-Flag/hGPIHBP1-Avi complex (50 nM stock in assay buffer, 10 nM complex final concentration) was added to 8 μL/well buffer. Then 4 μL/well 5×ANGPTL4(26-406)-6HIS-myc (final concentrations of 0.2 to 100 nM) was added and incubation was carried out at room temperature for 1 hour. After incubation 4 μL 5× anti-HA-Tb/anti-myc-D2 (Cisbio, diluted in assay buffer to 2.5 nM anti-HA-Tb/200 nM anti-myc-D2 stock concentration) (0.5 nM anti-HA-Tb/40 nM anti-myc-D2 final concentration) was added and the mixture was incubated at room temperature for 3 hours. Finally the plate was read on an Envision plate reader Ex=320 Em=665 Em=615.

### AlphaLISA Based Assay for Detection of LPL/ANGPTL4 Complex Disruption

The assay for AlphaLISA® based detection of LPL/ANGPTL4 complex disruption was carried out in 384-well plates (ProxiPlate™ 384-well white, Perkin Elmer, USA) in a final volume of 20 μl. The composition of the assay buffer was 20 mM HEPES pH=7.4, 100 mM NaCl, 10% HI-FBS, 5 mM CaCl₂). The assay procedure was as follows: First 4 μL/well of 5×HA-hLPL/hGPIHBP1 complex (5 nM stock in assay buffer) (1 nM complex final concentration) was added. Subsequently, 4 μL/well of 5×ANGPTL4(26-406)-6HIS-myc (50 nM stock in assay buffer, 10 nM final concentration) was added. After an incubation of 1 hour, 4 μL of 5× anti-HA AlphaLISA® acceptor beads (Perkin Elmer, 100 μg/mL in 1× immunoassay buffer, 20 μg/mL final concentration) was added and the mixture was incubated again for 1 hour at room temperature. Then 4 μL 5× anti-myc AlphaLISA® donor beads (Perkin Elmer custom bead coating, 100 μg/mL in 1× immunoassay buffer, 20 μg/mL final concentration) was added and plates were sealed with a TOP Seal-A plus clear plate seal (Perkin Elmer). The plates were incubated at room temperature overnight and were subsequently read on an Envision plate reader using default AlphaLISA® instrument settings. Depending on pipetting technique, brief centrifugation pulses at 1500 RPM were added after additions to insure that reagents reached the bottom of the wells.

### Epitope Mapping by Hydrogen-Deuterium Exchange/Mass Spectrometry

Hydrogen-deuterium exchange (HDx) in combination with mass spectrometry (MS) (Woods & Hamuro, 2001, J Cell Biochem, Suppl 37:89-98, the contents of which are incorporated herein for this purpose) was used to map the ANGPTL4 binding epitope on LPL protein. Automated HDx/MS experiments were performed using methods similar to those described in the literature (Chalmers, 2006, the contents of which are incorporated herein for this purpose). The experiments were performed on a Waters HDx-MS platform, which includes a LEAP autosampler, nanoACQUITY UPLC System, and Synapt G2 mass spectrometer. The LPL-GPIHBP1 fusion polypeptide (15.8 μM) in the absence as well as presence of ANGPTL4 (79.2 μM) was labeled in a deuterium Tris-HCl buffer at pH 7 for 15 minutes at 4° C. The labeling reaction was then quenched with chilled quench buffer on ice for three minutes. Next, the quenched protein solution was injected onto the LC-MS system for automated pepsin digestion and peptide analysis. The ANGPTL4 binding epitope on LPL-GPIHBP1 fusion polypeptide was mapped by comparing the LC-MS data of the fusion polypeptide alone and with ANGPTL4. All measurements were carried out using a minimum of three analytical triplicates.

### Animal Studies

Male, 12-week old C57BL/6 (Taconic, Rensselaer, N.Y.), 12-15-week old DBA/2J or 15-24-week old TALLYHO/JngJ mice (Jackson Lab, Bar Harbor, Me.) were used for the studies. Animals were housed in normal light cycle (6:00 am-6:00 pm), fed on normal chow or high fat high sucrose diet (Research diets cat #D12331i), and had access to water ad libitum during the studies. All procedures were in compliance with the Animal Welfare Act Regulations 9 CFR Parts 1, 2 and 3, and other guidelines. The studies were performed under an animal protocol approved by the Institutional Animal Care and Use Committee of Novartis Institutes for BioMedical Research. Blood samples were taken by tail vein bleeding, collected in Microvette tubes (Sarstedt AG & Co., Numbrecht, Germany) and kept on ice before centrifugation. Animals were randomly assigned into either vehicle or treatment groups (n=6-8/group) with serum triglyceride (TG) levels assessed using WAKO Diagnostics kit (Mountain View, Calif.) and matched among groups.

On the day of the study, animals were dosed intravenously (IV) or subcutaneously (SC) with human serum albumin (HSA, as negative control) or LPL-GPIHBP1 in PBS at doses from 0.3 to 30 mg/5 ml/kg. Tail blood samples were taken before dosing and at multiple time points after dosing. Serum TG levels were determined as described above.

Lipid tolerance test was performed in DBA/2J mice. Intralipid (A phospholipid-stabilized soybean oil as 20% fat emulsion, Sigma-Aldrich, St. Louis, Mo.) was injected IV. Serum samples were obtained from tail vein bleeding and measured for TG before and at 0.5, 1, and 2 hours after the Intralipid injection.

Statistical analysis was performed using GraphPad Prism 7.0 (GraphPad Software, San Diego, Calif.). Time course analysis was performed by a two-way analysis of variance (ANOVA) followed by a post-hoc test using Bonferroni's method for each time point. Data are presented as means±standard error of the mean (SEM). Statistical significance was accepted at the level of p<0.05.

### Example 1: GPIHBP1 Stabilizes LPL, Prevents its Aggregation, and Increases Lipase Activity

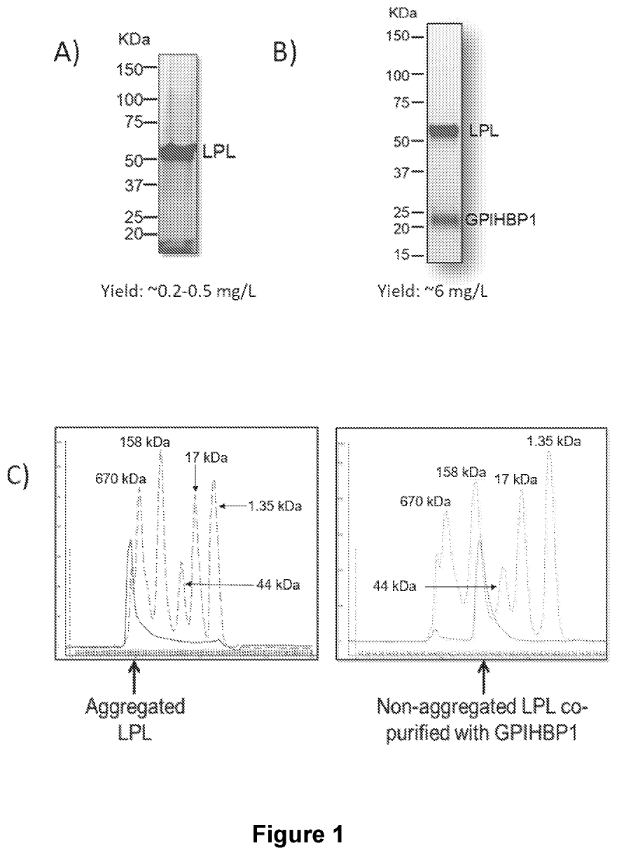

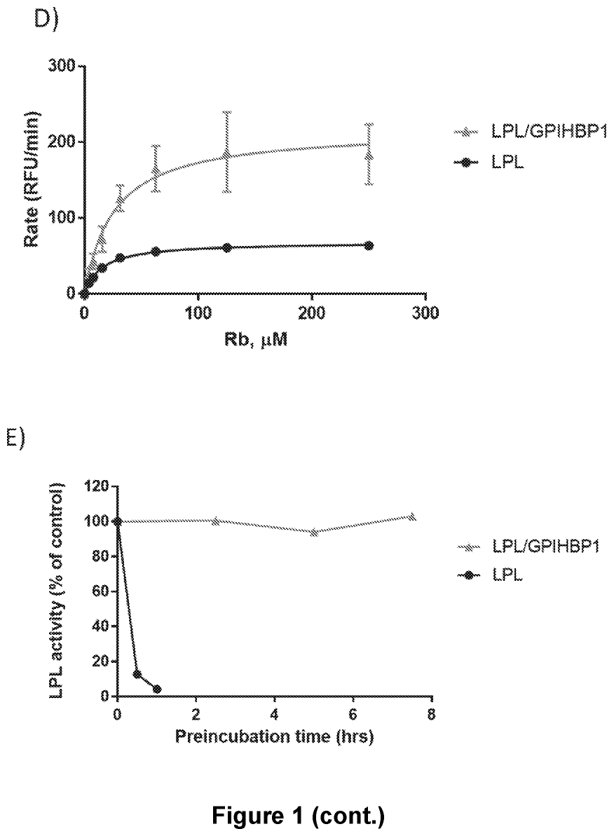

Initially an attempt was made to purify LPL protein by itself. In order to aid this purification, a variety of LPL constructs were synthesized that were either untagged or had N- or C-terminal tags. These LPL constructs were expressed in mammalian cells and purified using heparin chromatography or Ni-affinity chromatography. The purified protein was found to be active, but highly aggregated (FIG. 1, panels A, C, and D). Co-transfection of LPL with LMF1 did not improve LPL yield and the purified protein was still highly aggregated (data not shown). Co-expression of the purified soluble form of GPIHBP1 with LPL protected LPL against spontaneous inactivation (data not shown). GPIHBP1 was then co-expressed with LPL in the presence of LMF1 in the following manner: N-terminally 6-His tagged LPL, untagged GPIHBP1, and LMF1 were co-transfected in a ratio of 3:1:1 into HEK293T cells. The expressed protein complex was captured using Ni-affinity chromatography. This triple transfection significantly improved purity and yield of LPL (FIG. 1, panel B). Importantly, the presence of GPIHBP1 and LMF1 generated an LPL/GPIHBP1 complex that was homogenous and eluted as a ˜75 KDa complex during size exclusion chromatography (FIG. 1, panel C). This agreed well with the predicted molecular weight of a 1:1 LPL/GPHBP1 complex. The LPL/GPHBP1 complex also possessed ˜3-4-fold higher activity than LPL alone (FIG. 1, panel D) and was resistant to spontaneous inactivation of LPL (FIG. 1, panel E).

### Example 2: LPL-GPIHBP1 Fusion Polypeptide is Homogenous, Stable, and has High Specific Activity

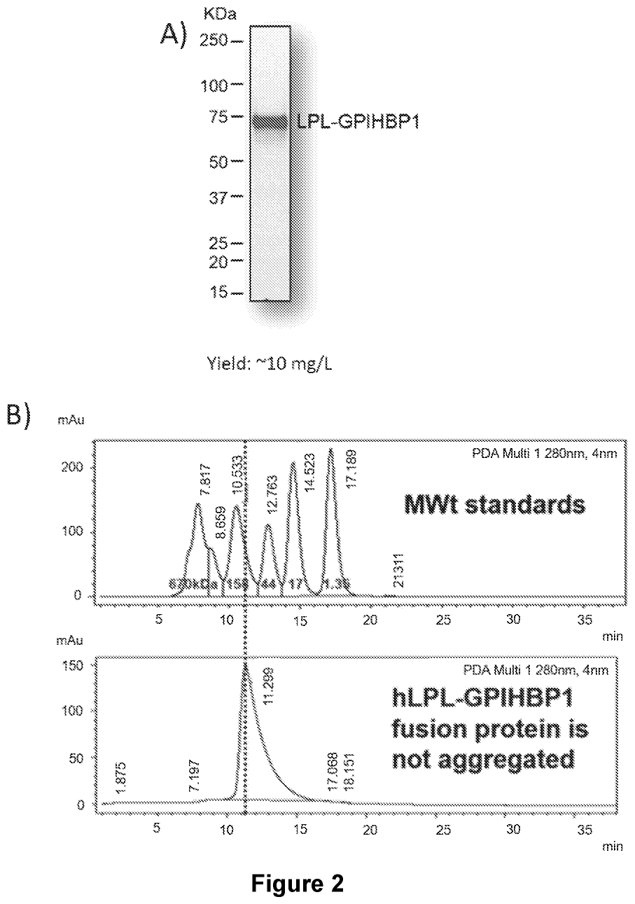

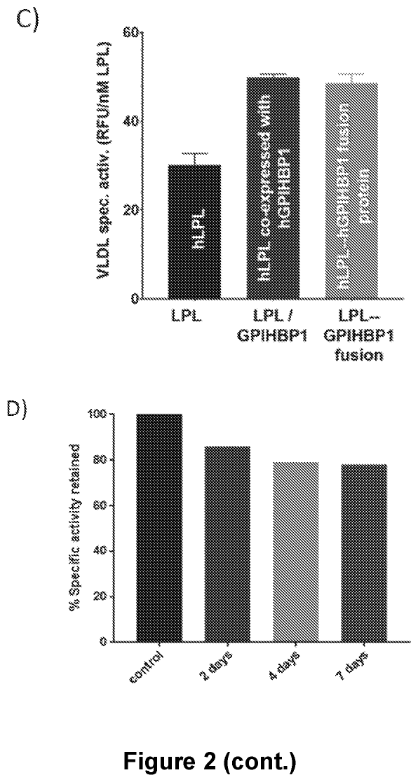

A non-dissociating complex of LPL and GPIHBP1 was then created by making an LPL-GPIHBP1 fusion construct. Mammalian expression vectors were designed that had LPL and soluble GPIHBP1 open reading frames connected via a 20 amino acid serine/glycine linker. To aid in purification, purification tags were added to either the N- or C-terminal ends of the fusion construct. FIG. 2, panel A shows the purified LPL-GPIHBP1 complex with a C-terminal FLAG-6-His-AviTag™ (FHA) tag. More than 95% pure fusion polypeptide was obtained using Ni-affinity and size exclusion chromatography (FIG. 2, panel A). Similarly to the LPL/GPIHBP1 co-expressed complex, the created fusion polypeptide was free of aggregates and resolved as a single homogenous species with a molecular weight of ˜75 KDa by size exclusion chromatography (FIG. 2, panel B). These data indicated that the LPL/GPIHBP1 complex as well as the fusion polypeptide consisted of 1 LPL and 1 GPIHBP1 molecule. Indeed, the crystal structure of LPL/GPIHBP1 complex confirmed that LPL and GPIHBP1 formed a 1:1 monomeric complex (data not shown). The fusion polypeptide had activity comparable with the co-purified LPL/GPIHBP1 complex (FIG. 2, panel C), thereby confirming that the fusion does not adversely affect the catalytic activity of LPL. Moreover, the fusion polypeptide was highly stable and maintained activity over a period of 7 days at 4 degrees (FIG. 2, panel D).

### Example 3: ANGPTL4 Dissociates LPL-GPIHBP1 Complex

ANGPTL4/LPL/GPIHBP1 interactions were then examined to determine if binding of ANGPTL4 to LPL lead to dissociation of GPIHBP1.

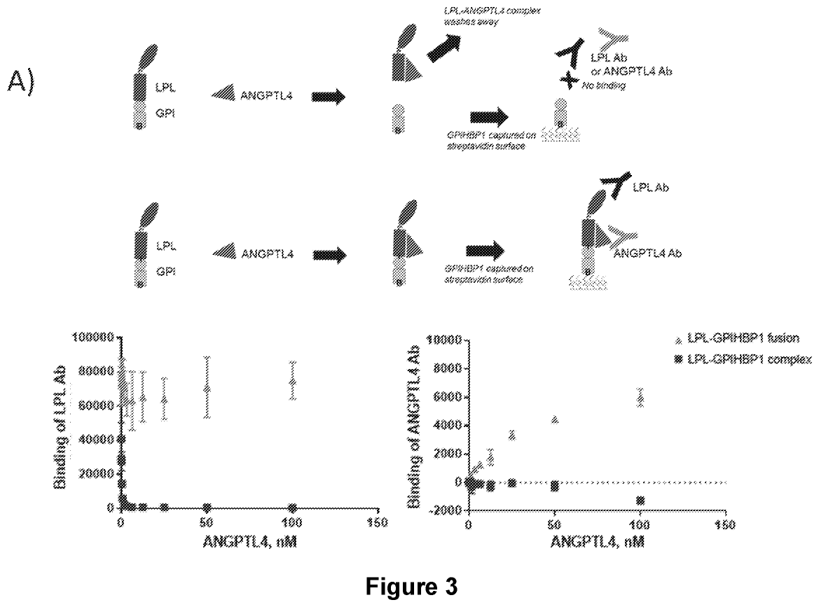

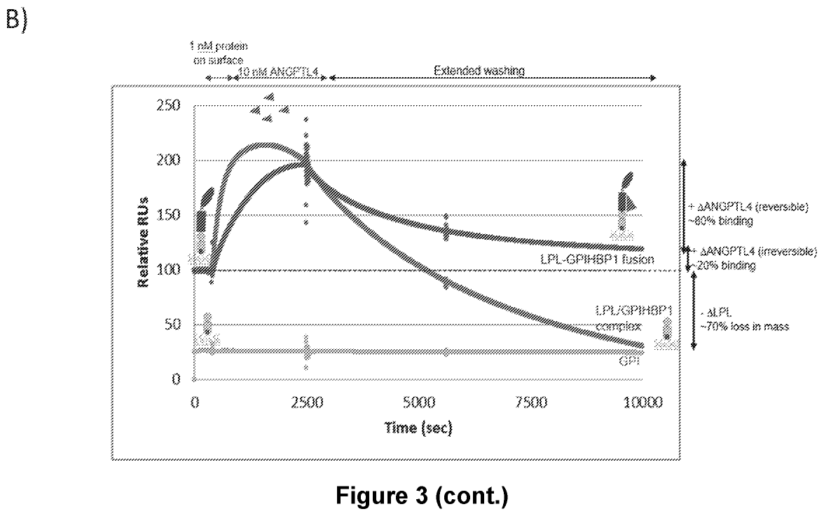

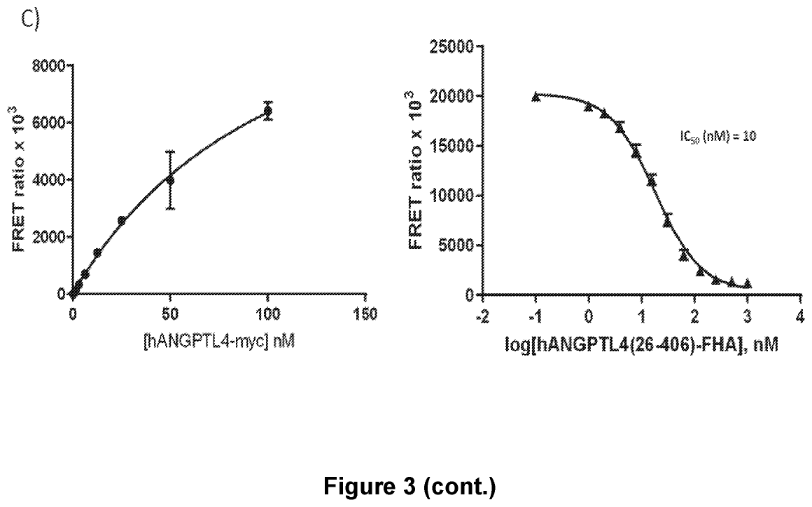

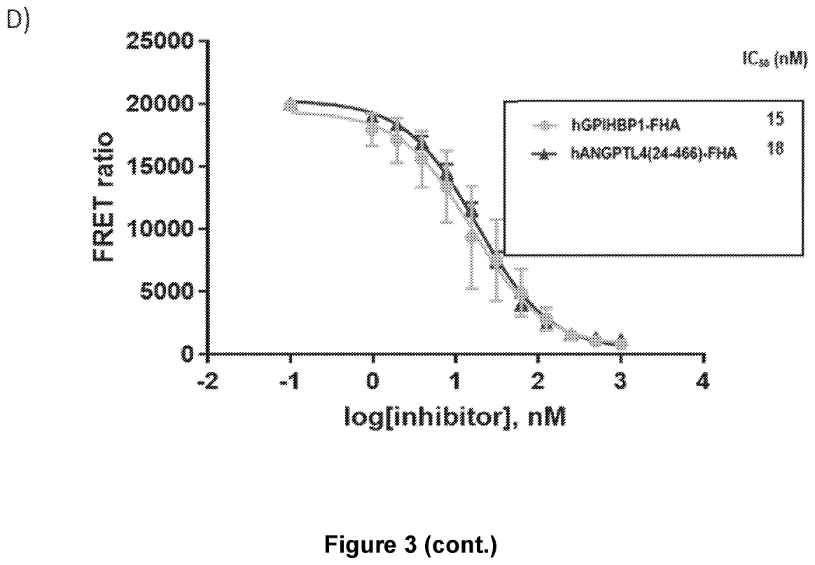

First, the co-expressed LPL/GPIHBP1 complex or the LPL-GPIHBP1 fusion polypeptide was immobilized on a streptavidin surface through C-terminally biotinylated GPIHBP1. The proteins were then incubated with ANGPTL4 and changes in bound ANGPTL4 and LPL were monitored with respective high affinity antibodies. A schematic representation of the assay is depicted on top of FIG. 3, panel A. When the complex was incubated with ANGPTL4, displacement of LPL from the co-expressed LPL/GPIHBP1 complex as function of ANGPTL4 concentration was observed. On the other hand, ANGPTL4 was unable to displace LPL from the covalently linked LPL-GPIHBP1 fusion polypeptide (FIG. 3, panel A, left side). Correspondingly, when bound ANGPTL4 was probed, ANGPTL4 was not captured by the co-expressed LPL/GPIHBP1 complex. However, ANGPTL4 accumulated on LPL covalently linked to GPIHBP1 as a function of ANGPTL4 concentration (FIG. 3, panel A, right side).

Next, the interaction of ANGPTL with LPL and GPIHBP1 was analyzed using SPR (FIG. 3, panel B). Displacement of LPL by ANGPTL4 from co-expressed LPL/GPIHBP1 complex, but not from the fusion polypeptide, was confirmed.

Finally, in a TR-FRET assay, ANGPTL4 was observed to bind to the LPL-GPIHBP1 fusion polypeptide (FIG. 3, panel C, left side) and dissociate the LPL/GPIHBP1 complex (FIG. 3, panel C, right side. ANGPTL3 was also able to dissociate the complex, albeit not as efficiently as ANGPTL4 (data not shown).

These data indicated that binding of ANGPTL4 and GPIHBP1 to LPL is mutually exclusive. Indeed, when the LPL/GPIHBP1 complex was challenged with free GPIHBP1 or ANGPTL4, both proteins were able to dissociate the complex with similar IC₅₀ values (FIG. 3, panel D).

These observations were consistent with functional competition of ANGPTL4 and GPIHBP1 for LPL. Hence, we speculated that LPL-GPIHBP1 fusion polypeptide may be more resistant to ANGPTL4 inactivation than free LPL and co-expressed LPL/GPIHBP1 complex.

### Example 4: LPL-GPIHBP1 Fusion is Resistant to Inactivation by ANGPTL4 and ANGPTL3

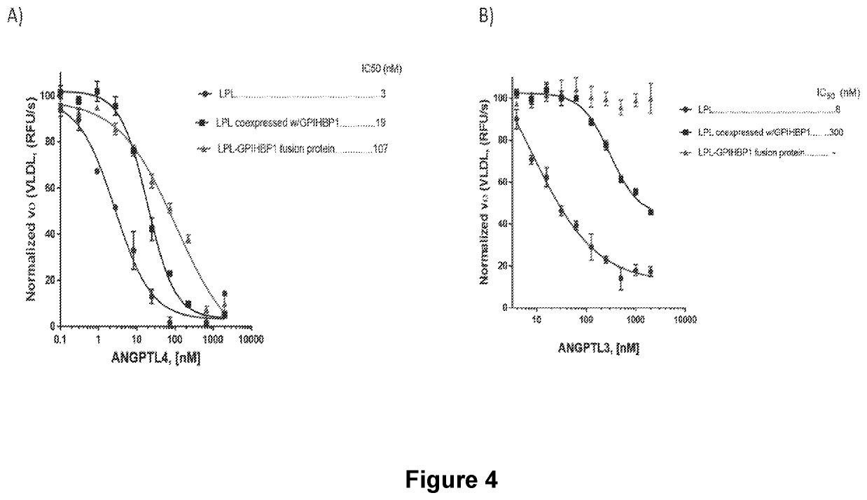

The effect of ANGPTL3 and ANGPTL4 on the enzymatic activity of LPL was investigated. LPL/GPIHBP1 co-expressed complex was observed to be more than 6-fold more resistant to ANGPTL4 inactivation than LPL alone (IC₅₀ 19 nM and 3 nM respectively) (FIG. 4, panel A).

Similarly, the LPL/GPIHBP1 co-expressed complex was also more resistant (>37 fold) to ANGPTL3 inactivation than LPL alone (IC₅₀ 300 nM and 8 nM respectively) (FIG. 4, panel B). This indicated that GPIHBP1 not only protected LPL from spontaneous inactivation, but also stabilized it against inactivation by ANGPTLs. This stabilizing effect was even more pronounced in the LPL-GPIHBP1 fusion polypeptide. The IC₅₀ for ANGPTL4 mediated inactivation of the fusion polypeptide was more than 35-fold higher than that of LPL alone and more than 5-fold higher than the co-expressed LPL/GPIHBP1 complex (107 nM, 3 nM, and 19 nM respectively) (FIG. 4, panel A). The stabilizing effect of GPIHBP1 against ANGPTL3 inactivation was also more pronounced for the fusion polypeptide. The IC₅₀ for LPL alone and the LPL/GPIHBP1 co-expressed complex were 7 nM and 300 nM respectively, while no loss of activity by the LPL-GPIHBP1 fusion polypeptide was observed (FIG. 4, panel B).

### Example 5: HDx-MS Experiment Map the Binding Epitope of ANGPTL4 on LPL

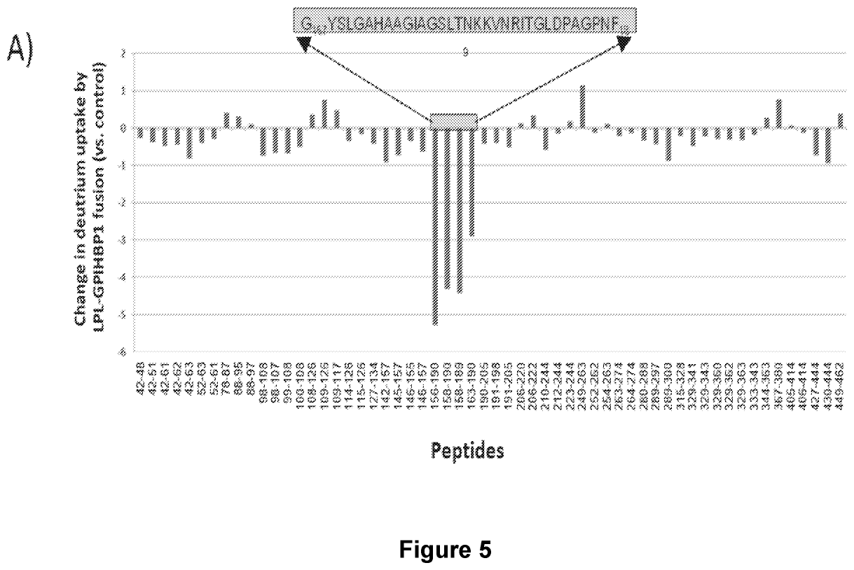

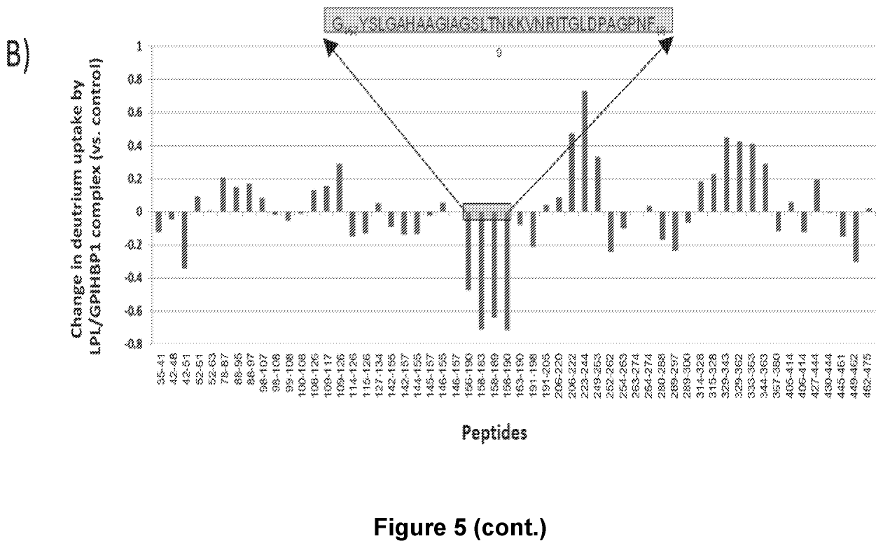

Our studies thus far demonstrated that ANGPTL4 mediated inactivation of the fusion polypeptide was weaker than that of the co-expressed LPL/GPIHBP1 complex, and that ANGPTL4 stayed bound to LPL. The fusion polypeptide was therefore used to map the ANGPTL4 binding site on LPL. For this purpose, hydrogen-deuterium exchange (HDx) was utilized in combination with mass spectrometry (MS). In this method, the covalently bonded hydrogen atoms were replaced with deuterium. The exchange reaction was performed with isolated proteins and with the protein complex. The two reactions were then compared. If a region was buried in the protein complex, the amides in this region were expected to exchange more slowly in comparison to the proteins in isolation. Using this method, d a stretch of 32 amino acids (amino acids 157 to 189 of SEQ ID NO: 1) was identified in the LPL-GPIHBP1 fusion polypeptide that was shielded by the presence of ANGPTL4 (FIG. 5, panel A). The same stretch of amino acids was also protected in the co-expressed LPL/GPIHBP1 complex (FIG. 5, panel B). This suggested that the interaction of LPL and ANGPTL4 was not fundamentally altered by the fusion of LPL to GPIHBP1. The knowledge of amino acids involved in LPL/ANGPTL4 interactions provided a roadmap for further stabilization of the LPL-GPIHBP1 fusion polypeptide through LPL site-directed mutagenesis.

### Example 6: LPL-GPIHBP1 Fusion Polypeptide Lowered Triglyceride Level in Several Strains of Mice

The improved pharmacological properties of LPL-GPIHBP1 fusion polypeptide (low aggregation, resistance to spontaneous inactivation, high purification yields) as well as resistance of the fusion polypeptide to inactivation by LPL antagonists ANGPTL3 and ANGPTL4 made us speculate that the fusion polypeptide could be an effective therapeutic for acute TG lowering in vivo. Mice are a convenient animal model for such studies, but plasma TG can vary significantly between mouse strains. The TG lowering effect of LPL-GPIHBP1 was therefore tested in several strains of mice to ensure that the observed effects are not strain-specific.

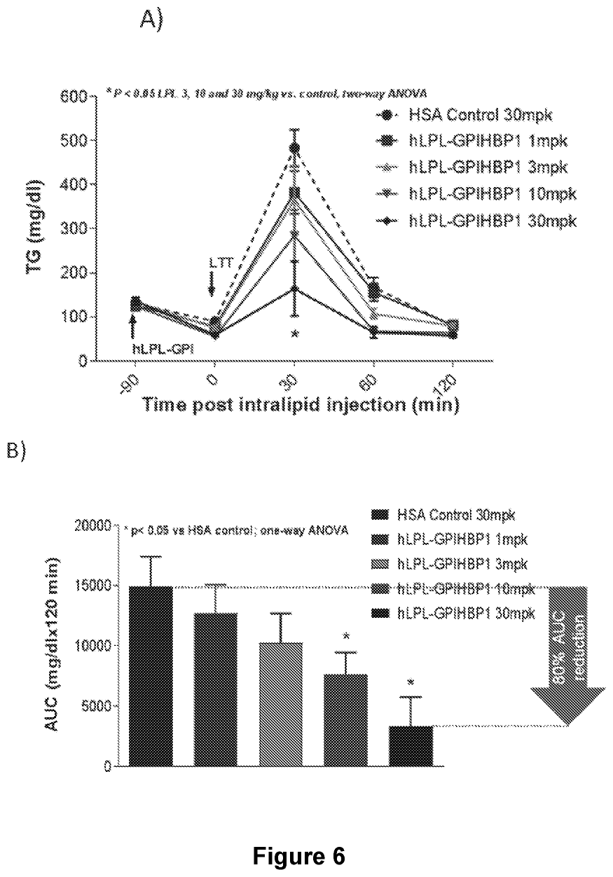

TG lowering was first tested in C57BL/6 mice. Since these mice have intrinsically low basal TG of (˜100 mg/dL), their plasma TG was transiently increased with a bolus of intralipid (Lipid tolerance test, FIG. 6). As FIG. 6 indicates, this led to a ˜5 fold spike in TG levels ˜30 min post injection of intralipid bolus (FIG. 6, HSA control). Importantly, SC administered LPL-GPIHBP1 dose-dependently blunted the TG increase, with the highest dose lowering the AUC of TG excursion by ˜80% (FIG. 6, panels A and B).

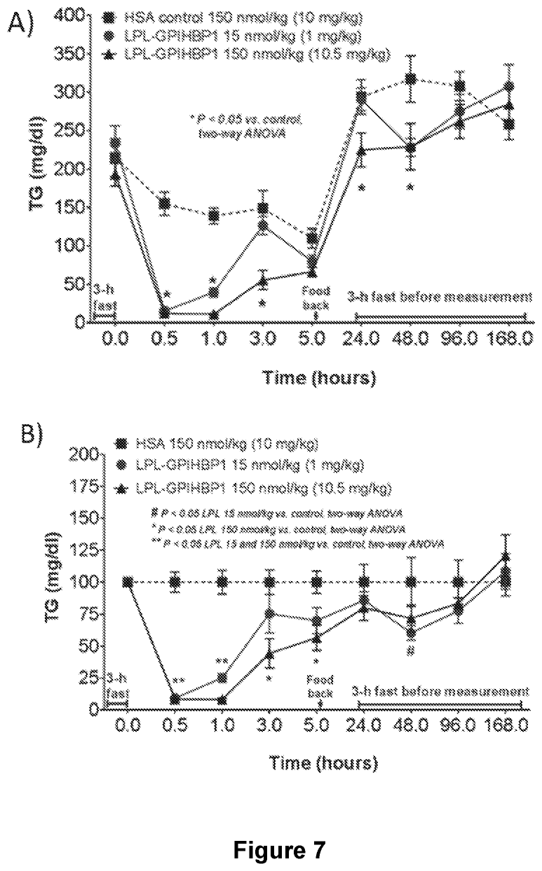

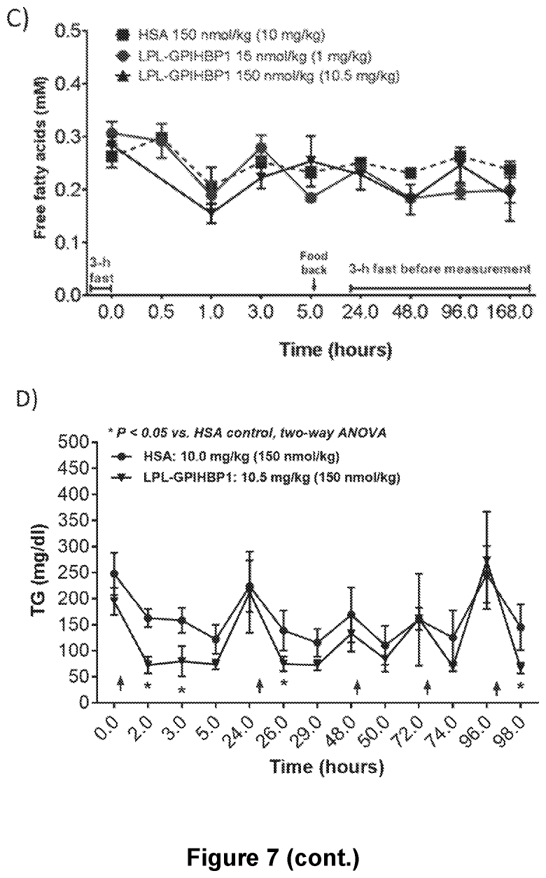

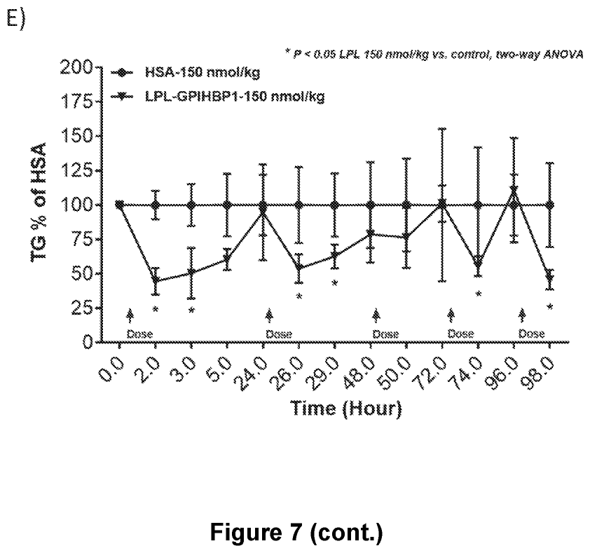

Next, the effect of the fusion polypeptide on TG levels was tested in DBA/2 mice, who have higher basal TG of ˜200 mg/dL. Since TG levels in mice plasma may change substantially over the course of the day (in some cases demonstrating a 50-60% decrease during the non-feeding period), fusion polypeptide mediated reduction in TG levels was normalized to an HSA control administered as part of the same study. Here as well, LPL-GPIHBP1 dose-dependently lowered TG in DBA/2 mice after IV administration with more than 90% TG reduction at the highest dose (FIG. 7, panels A and B). One of the concerns is that fast TG lowering of such magnitude may lead to an increase in pro-inflammatory free fatty acids in plasma. No increase in plasma free fatty acids was observed (FIG. 7, panel C), indicating that hydrolyzed TG were utilized by the tissues. LPL-GPIHBP1 was also administered to DBA/2 mice daily for 5 days. The fusion polypeptide consistently decreased plasma TG (FIG. 7, panels D and E) without overt TG accumulation in the liver, heart, skeletal muscle, or fat tissue.

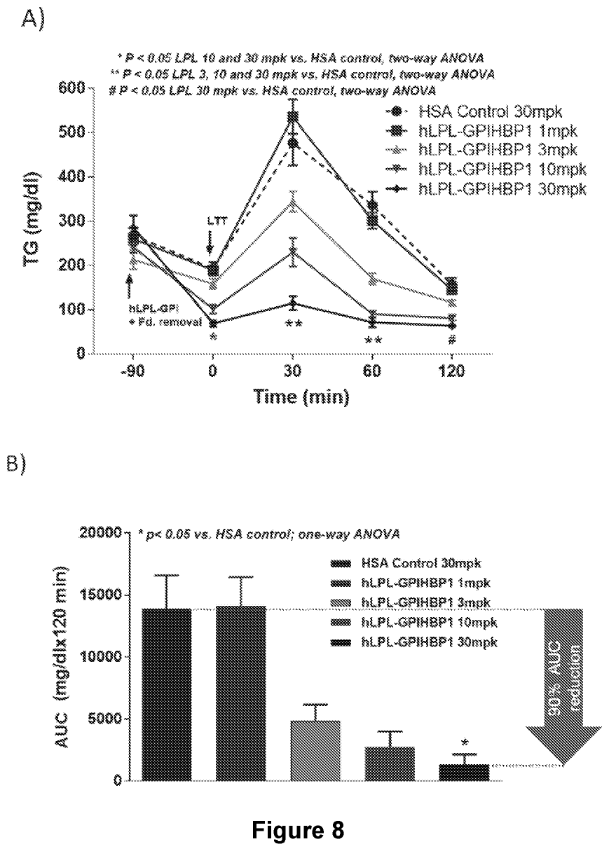

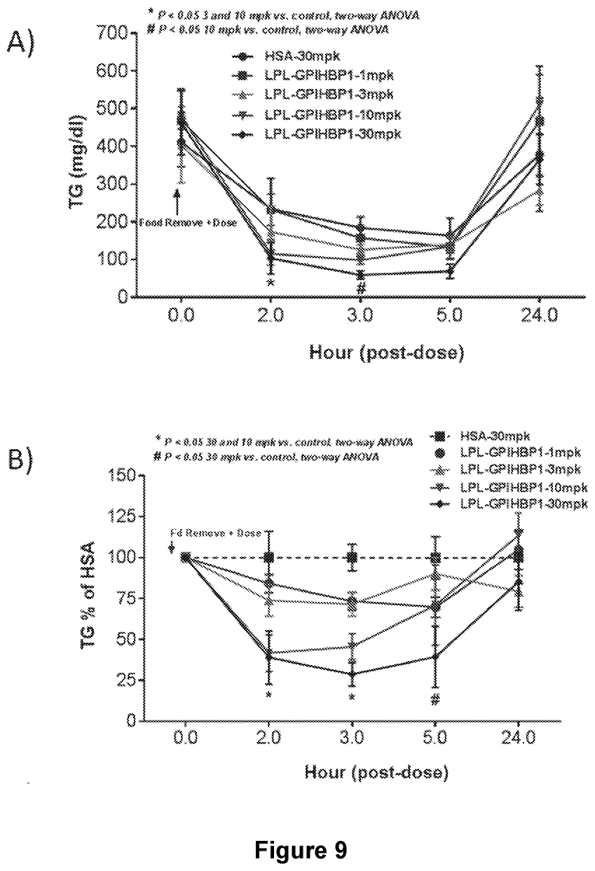

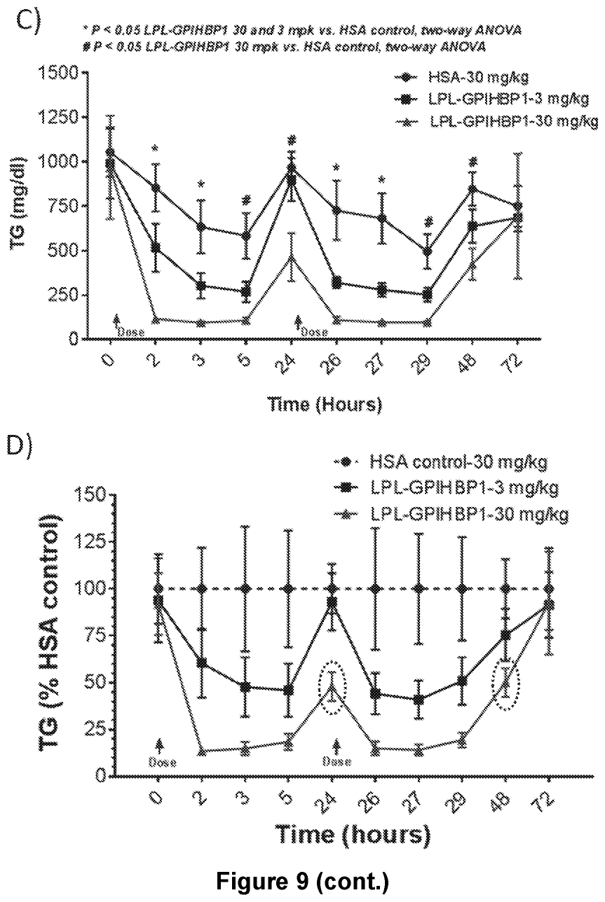

To achieve higher plasma TG, LPL-GPIHBP1 was administered SC to DBA/2 mice during the lipid tolerance test. In agreement with the results of the C57BL/6 study, the LPL-GPIHBP1 fusion polypeptide dose-dependently blunted the TG increase with the highest dose lowering the AUC of TG excursion by ˜90% (FIG. 8, panels A and B). Finally, the effect of LPL-GPIHBP1 on TG lowering was tested in TALLYHO mice, a strain with the baseline TG of ˜400 mg/dL. In this hyperlipidemic strain, sub-cutaneous administration of LPL-GPIHBP1 dose-dependently lowered TG, with the highest dose lowering TG by ˜70% (FIG. 9, panels A and B). To achieve even higher basal plasma TG, TALLYHO mice were maintained on a high fat/high sucrose (HFHS) diet. This regimen increased their TG to ˜1000 mg/dL (approaching levels similar to those seen in human patients with FCS). Repeat SC administered LPL-GPIHBP1 again dose-dependently decreased plasma TG with the highest dose leading to ˜90% TG lowering (FIG. 9, panels C and D). These data convincingly demonstrated that the LPL-GPIHBP1 fusion polypeptide acutely lowered TG in vivo.

### Example 7: Addition of Albumin-Binding Moiety to LPL-GPIHBP1 Fusion Prolonged Duration of Triglyceride Lowering

LPL-GPIHBP1 was determined to have a plasma half-life of approximately 20 min after IV administration and of approximately 1.7 hours when administered SC (data not shown), thereby explaining the transient lowering of TG for 2-5 hours after protein administration in a variety of mouse models. To prolong the TG-lowering effect, a variant of the LPL-GPIHBP1 fusion polypeptide was generated by attaching the Fab domain of CA645 (Adams et al., 2016, supra) to the N-terminus of LPL-GPIHBP1. In another construct, a CA645 scFv was attached to the C-terminus of LPL-GPIHBP1. Both constructs bound albumin (data not shown) and confirmed the modifications did not compromise LPL enzymatic activity of either construct by using the enzyme's natural substrate VLDL.

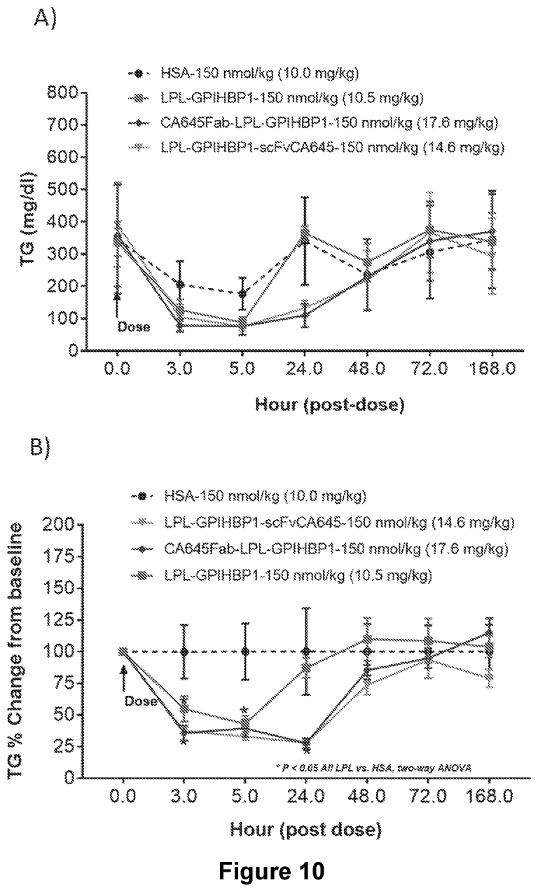

The ability of these constructs to lower TG in DBA/2 mice was then tested. Attachment of albumin-binding moieties increased the duration of maximum TG-lowering from approximately 3 to 24 hours (FIG. 10).

SEQUENCE LISTING
(Human LPL; UniProtKB/Swiss-Prot: P06858.1)
SEQ ID NO: 1
MESKALLVLTLAVWLQSLTASRGGVAAADQRRDFIDIESKFALRTPEDTAEDTCHLIPGVAESVATCHFNHSSKTFM
VIHGWTVTGMYESWVPKLVAALYKREPDSNVIVVDWLSRAQEHYPVSAGYTKLVGQDVARFINWMEEEFNYPLDNVH
LLGYSLGAHAAGIAGSLTNKKVNRITGLDPAGPNFEYAEAPSRLSPDDADFVDVLHTFTRGSPGRSIGIQKPVGHVD
IYPNGGTFQPGCNIGEAIRVIAERGLGDVDQLVKCSHERSIHLFIDSLLNEENPSKAYRCSSKEAFEKGLCLSCRKN
RCNNLGYEINKVRAKRSSKMYLKTRSQMPYKVFHYQVKIHFSGTESETHTNQAFEISLYGTVAESENIPFTLPEVST
NKTYSFLIYTEVDIGELLMLKLKWKSDSYFSWSDWWSSPGFAIQKIRVKAGETQKKVIFCSREKVSHLQKGKAPAVF
VKCHDKSLNKKSG
(Mature human LPL - amino acids 28-475)
SEQ ID NO: 2
ADQRRDFIDIESKFALRTPEDTAEDTCHLIPGVAESVATCHFNHSSKTFMVIHGWTVTGMYESWVPKLVAALYKREP
DSNVIVVDWLSRAQEHYPVSAGYTKLVGQDVARFINWMEEEFNYPLDNVHLLGYSLGAHAAGIAGSLTNKKVNRITG
LDPAGPNFEYAEAPSRLSPDDADFVDVLHTFTRGSPGRSIGIQKPVGHVDIYPNGGTFQPGCNIGEAIRVIAERGLG
DVDQLVKCSHERSIHLFIDSLLNEENPSKAYRCSSKEAFEKGLCLSCRKNRCNNLGYEINKVRAKRSSKMYLKTRSQ
MPYKVFHYQVKIHFSGTESETHTNQAFEISLYGTVAESENIPFTLPEVSTNKTYSFLIYTEVDIGELLMLKLKWKSD
SYFSWSDWWSSPGFAIQKIRVKAGETQKKVIFCSREKVSHLQKGKAPAVFVKCHDKSLNKKSG
(Mature human LPL with “S447X” mutation)
SEQ ID NO: 3
ADQRRDFIDIESKFALRTPEDTAEDTCHLIPGVAESVATCHFNHSSKTFMVIHGWTVTGMYESWVPKLVAALYKREP
DSNVIVVDWLSRAQEHYPVSAGYTKLVGQDVARFINWMEEEFNYPLDNVHLLGYSLGAHAAGIAGSLTNKKVNRITG
LDPAGPNFEYAEAPSRLSPDDADFVDVLHTFTRGSPGRSIGIQKPVGHVDIYPNGGTFQPGCNIGEAIRVIAERGLG
DVDQLVKCSHERSIHLFIDSLLNEENPSKAYRCSSKEAFEKGLCLSCRKNRCNNLGYEINKVRAKRSSKMYLKTRSQ
MPYKVFHYQVKIHFSGTESETHTNQAFEISLYGTVAESENIPFTLPEVSTNKTYSFLIYTEVDIGELLMLKLKWKSD
SYFSWSDWWSSPGFAIQKIRVKAGETQKKVIFCSREKVSHLQKGKAPAVFVKCHDKSLNKK
(amino acids 37-334 of SEQ ID NO: 1; minimal LPL catalytic domain)
SEQ ID NO: 4
IESKFALRTPEDTAEDTCHLIPGVAESVATCHFNHSSKTFMVIHGWTVTGMYESWVPKLVAALYKREPDSNVIVVDW
LSRAQEHYPVSAGYTKLVGQDVARFINWMEEEFNYPLDNVHLLGYSLGAHAAGIAGSLTNKKVNRITGLDPAGPNFE
YAEAPSRLSPDDADFVDVLHTFTRGSPGRSIGIQKPVGHVDIYPNGGTFQPGCNIGEAIRVIAERGLGDVDQLVKCS
HERSIHLFIDSLLNEENPSKAYRCSSKEAFEKGLCLSCRKNRCNNLGYEINKVRAKRSSKMYLKTRS
(Human GPIHBP1; UniProtKB: Q8IV16)
SEQ ID NO: 5
MKALGAVLLALLLCGRPGRGQTQQEEEEEDEDHGPDDYDEEDEDEVEEEETNRLPGGRSRVLLRCYTCKSLPRDERC
NLTQNCSHGQTCTTLIAHGNTESGLLTTHSTWCTDSCQPITKTVEGTQVTMTCCQSSLCNVPPWQSSRVQDPTGKGA
GGPRGSSETVGAALLLNLLAGLGAMGARRP
(Mature human GPIHBP1 - amino acids 21-184)
SEQ ID NO: 6
QTQQEEEEEDEDHGPDDYDEEDEDEVEEEETNRLPGGRSRVLLRCYTCKSLPRDERCNLTQNCSHGQTCTTLIAHGN
TESGLLTTHSTWCTDSCQPITKTVEGTQVTMTCCQSSLCNVPPWQSSRVQDPTGKGAGGPRGSSETVGAALLLNLLA
GLGAMGARRP
(human GPIHBP1 - no signal sequence, no propeptide - amino acids 21-151)
SEQ ID NO: 7
QTQQEEEEEDEDHGPDDYDEEDEDEVEEEETNRLPGGRSRVLLRCYTCKSLPRDERCNLTQNCSHGQTCTTLIAHGN
TESGLLTTHSTWCTDSCQPITKTVEGTQVTMTCCQSSLCNVPPWQSSRVQDPTG
(human GPIHBP1 - no propeptide - amino acids 1-151)
SEQ ID NO: 8
MKALGAVLLALLLCGRPGRGQTQQEEEEEDEDHGPDDYDEEDEDEVEEEETNRLPGGRSRVLLRCYTCKSLPRDERC
NLTQNCSHGQTCTTLIAHGNTESGLLTTHSTWCTDSCQPITKTVEGTQVTMTCCQSSLCNVPPWQSSRVQDPTG
(minimal functional domain human GPIHBP1 - amino acids 63-148)
SEQ ID NO: 9
LRCYTCKSLPRDERCNLTQNCSHGQTCTTLIAHGNTESGLLTTHSTWCTDSCQPITKTVEGTQVTMTCCQSSLCNVP
PWQSSRVQD
(truncated human GPIHBP1 - amino acids 21-160)
SEQ ID NO: 10
QTQQEEEEEDEDHGPDDYDEEDEDEVEEEETNRLPGGRSRVLLRCYTCKSLPRDERCNLTQNCSHGQTCTTLIAHGN
TESGLLTTHSTWCTDSCQPITKTVEGTQVTMTCCQSSLCNVPPWQSSRVQDPTGKGAGGPRGS
(linker)
SEQ ID NO: 11
GSG
(linker)
SEQ ID NO: 12
GSGGG
(linker)
SEQ ID NO: 13
GSGG
(linker)
SEQ ID NO: 14
SGGG
(linker)
SEQ ID NO: 15
GGGGS
(linker)
SEQ ID NO: 16
GGGGSGGGGSGGGGSGGGGS
(linker)
SEQ ID NO: 17
GGGGSGGGGSGGGGS
(linker)
SEQ ID NO: 18
GPPGS
(linker)
SEQ ID NO: 19
GGGS
(linker)
SEQ ID NO: 20
GYS
(linker)
SEQ ID NO: 21
GS
(linker)
SEQ ID NO: 22
SGGGG
(linker)
SEQ ID NO: 23
SGG
(linker)
SEQ ID NO: 24
SG
(linker)
SEQ ID NO: 25
GGGGA
(linker)
SEQ ID NO: 26
GGGA
(linker)
SEQ ID NO: 27
EAAAK
(6Histag)
SEQ ID NO: 28
HHHHHH
(FLAG tag)
SEQ ID NO: 29
DYKDDDDK
(Avitag)
SEQ ID NO: 30
GLNDIFEAQKIEWHE
(AHF tag)
SEQ ID NO: 31
GLNDIFEAQKIEWHEGGHHHHHHDYKDDDDK
(FHAtag)
SEQ ID NO: 32
DYKDDDDKHHHHHHGGGLNDIFEAQKIEWHE
(AHF-hLPL(28-475)-(GGGGS)₄-hGPIHBP1(21-151))
SEQ ID NO: 33
METDTLLLWVLLLWVPGSTGGLNDIFEAQKIEWHEGGHHHHHHDYKDDDDKADQRRDFIDIESKFALR
TPEDTAEDTCHLIPGVAESVATCHFNHSSKTFMVIHGWTVTGMYESWVPKLVAALYKREPDSNVIVVD
WLSRAQEHYPVSAGYTKLVGQDVARFINWMEEEFNYPLDNVHLLGYSLGAHAAGIAGSLTNKKVNRIT
GLDPAGPNFEYAEAPSRLSPDDADFVDVLHTFTRGSPGRSIGIQKPVGHVDIYPNGGTFQPGCNIGEAIR
VIAERGLGDVDQLVKCSHERSIHLFIDSLLNEENPSKAYRCSSKEAFEKGLCLSCRKNRCNNLGYEINKV
RAKRSSKMYLKTRSQMPYKVFHYQVKIHFSGTESETHTNQAFEISLYGTVAESENIPFTLPEVSTNKTYSF
LIYTEVDIGELLMLKLKWKSDSYFSWSDWWSSPGFAIQKIRVKAGETQKKVIFCSREKVSHLQKGKAPA
VFVKCHDKSLNKKSGGGGGSGGGGSGGGGSGGGGSQTQQEEEEEDEDHGPDDYDEEDEDEVEEEE
TNRLPGGRSRVLLRCYTCKSLPRDERCNLTQNCSHGQTCTTLIAHGNTESGLLTTHSTWCTDSCQPITK
TVEGTQVTMTCCQSSLCNVPPWQSSRVQDPTG
(AHF-hLPL(28-475)-(GGGGS)4-hGPIHBP1(21-151)(no signal sequence))
SEQ ID NO: 34
GLNDIFEAQKIEWHEGGHHHHHHDYKDDDDKADQRRDFIDIESKFALRTPEDTAEDTCHLIPGVAESV
ATCHFNHSSKTFMVIHGWTVTGMYESWVPKLVAALYKREPDSNVIVVDWLSRAQEHYPVSAGYTKLVG
QDVARFINWMEEEFNYPLDNVHLLGYSLGAHAAGIAGSLTNKKVNRITGLDPAGPNFEYAEAPSRLSPD
DADFVDVLHTFTRGSPGRSIGIQKPVGHVDIYPNGGTFQPGCNIGEAIRVIAERGLGDVDQLVKCSHER
SIHLFIDSLLNEENPSKAYRCSSKEAFEKGLCLSCRKNRCNNLGYEINKVRAKRSSKMYLKTRSQMPYKV
FHYQVKIHFSGTESETHTNQAFEISLYGTVAESENIPFTLPEVSTNKTYSFLIYTEVDIGELLMLKLKWKSD
SYFSWSDWWSSPGFAIQKIRVKAGETQKKVIFCSREKVSHLQKGKAPAVFVKCHDKSLNKKSGGGGG
SGGGGSGGGGSGGGGSQTQQEEEEEDEDHGPDDYDEEDEDEVEEEETNRLPGGRSRVLLRCYTCKSL
PRDERCNLTQNCSHGQTCTTLIAHGNTESGLLTTHSTWCTDSCQPITKTVEGTQVTMTCCQSSLCNVPP
WQSSRVQDPTG
(AHF-hLPL(28-475)-(G4S)4-hGPIHBP1(21-151)-(G4S)3-PAS200)
SEQ ID NO: 35
METDTLLLWVLLLWVPGSTGGLNDIFEAQKIEWHEGGHHHHHHDYKDDDDKADQRRDFIDIESKFALR
TPEDTAEDTCHLIPGVAESVATCHFNHSSKTFMVIHGWTVTGMYESWVPKLVAALYKREPDSNVIVVD
WLSRAQEHYPVSAGYTKLVGQDVARFINWMEEEFNYPLDNVHLLGYSLGAHAAGIAGSLTNKKVNRIT
GLDPAGPNFEYAEAPSRLSPDDADFVDVLHTFTRGSPGRSIGIQKPVGHVDIYPNGGTFQPGCNIGEAIR
VIAERGLGDVDQLVKCSHERSIHLFIDSLLNEENPSKAYRCSSKEAFEKGLCLSCRKNRCNNLGYEINKV
RAKASSKMYLKTRSQMPYKVFHYQVKIHFSGTESETHTNQAFEISLYGTVAESENIPFTLPEVSTNKTYSF
LIYTEVDIGELLMLKLKWKSDSYFSWSDWWSSPGFAIQKIRVKAGETQKKVIFCSREKVSHLQKGKAPA
VFVKCHDKSLNKKSGGGGGSGGGGSGGGGSGGGGSQTQQEEEEEDEDHGPDDYDEEDEDEVEEEE
TNRLPGGRSRVLLRCYTCKSLPRDERCNLTQNCSHGQTCTTLIAHGNTESGLLTTHSTWCTDSCQPITK
TVEGTQVTMTCCQSSLCNVPPWQSSRVQDPTGGGGGSGGGGSGGGGSSSAAASSSASPAAPAPASP
AAPAPSAPAASPAAPAPASPAAPAPSAPAASPAAPAPASPAAPAPSAPAASPAAPAPASPAAPAPSAPAAS
PAAPAPASPAAPAPSAPAASPAAPAPASPAAPAPSAPAASPAAPAPASPAAPAPSAPAASPAAPAPASPAA
PAPSAPAASPAAPAPASPAAPAPSAPAASPAAPAPASPAAPAPSAPAAHHHHHH
(AHF-hLPL(28-475)-(G4S)4-hGPIHBP1(21-151)-(G4S)3-PAS600)
SEQ ID NO: 36
METDTLLLWVLLLWVPGSTGGLNDIFEAQKIEWHEGGHHHHHHDYKDDDDKADQRRDFIDIESKFALR
TPEDTAEDTCHLIPGVAESVATCHFNHSSKTFMVIHGWTVTGMYESWVPKLVAALYKREPDSNVIVVD
WLSRAQEHYPVSAGYTKLVGQDVARFINWMEEEFNYPLDNVHLLGYSLGAHAAGIAGSLTNKKVNRIT
GLDPAGPNFEYAEAPSRLSPDDADFVDVLHTFTRGSPGRSIGIQKPVGHVDIYPNGGTFQPGCNIGEAIR
VIAERGLGDVDQLVKCSHERSIHLFIDSLLNEENPSKAYRCSSKEAFEKGLCLSCRKNRCNNLGYEINKV
RAKASSKMYLKTRSQMPYKVFHYQVKIHFSGTESETHTNQAFEISLYGTVAESENIPFTLPEVSTNKTYSF
LIYTEVDIGELLMLKLKWKSDSYFSWSDWWSSPGFAIQKIRVKAGETQKKVIFCSREKVSHLQKGKAPA
VFVKCHDKSLNKKSGGGGGSGGGGSGGGGSGGGGSQTQQEEEEEDEDHGPDDYDEEDEDEVEEEE
TNRLPGGRSRVLLRCYTCKSLPRDERCNLTQNCSHGQTCTTLIAHGNTESGLLTTHSTWCTDSCQPITK
TVEGTQVTMTCCQSSLCNVPPWQSSRVQDPTGGGGGSGGGGSGGGGSSSAAASSSASPAAPAPASP
AAPAPSAPAASPAAPAPASPAAPAPSAPAASPAAPAPASPAAPAPSAPAASPAAPAPASPAAPAPSAPAAS
PAAPAPASPAAPAPSAPAASPAAPAPASPAAPAPSAPAASPAAPAPASPAAPAPSAPAASPAAPAPASPAA
PAPSAPAASPAAPAPASPAAPAPSAPAASPAAPAPASPAAPAPSAPAASPAAPAPASPAAPAPSAPAASPA
APAPASPAAPAPSAPAASPAAPAPASPAAPAPSAPAASPAAPAPASPAAPAPSAPAASPAAPAPASPAAPA
PSAPAASPAAPAPASPAAPAPSAPAASPAAPAPASPAAPAPSAPAASPAAPAPASPAAPAPSAPAASPAAP
APASPAAPAPSAPAASPAAPAPASPAAPAPSAPAASPAAPAPASPAAPAPSAPAASPAAPAPASPAAPAPS
APAASPAAPAPASPAAPAPSAPAASPAAPAPASPAAPAPSAPAASPAAPAPASPAAPAPSAPAASPAAPAP
ASPAAPAPSAPAASPAAPAPASPAAPAPSAPAASPAAPAPASPAAPAPSAPAASPAAPAPASPAAPAPSAP
AASPAAPAPASPAAPAPSAPAAHHHHHH
(AHF-hLPL(28-475)-(G4S)4-hGPIHBP1(21-151)-(G4S)3-CA645scFv-LV-HV)
SEQ ID NO: 37
METDTLLLWVLLLWVPGSTGGLNDIFEAQKIEWHEGGHHHHHHDYKDDDDKADQRRDFIDIESKFALR
TPEDTAEDTCHLIPGVAESVATCHFNHSSKTFMVIHGWTVTGMYESWVPKLVAALYKREPDSNVIVVD
WLSRAQEHYPVSAGYTKLVGQDVARFINWMEEEFNYPLDNVHLLGYSLGAHAAGIAGSLTNKKVNRIT
GLDPAGPNFEYAEAPSRLSPDDADFVDVLHTFTRGSPGRSIGIQKPVGHVDIYPNGGTFQPGCNIGEAIR
VIAERGLGDVDQLVKCSHERSIHLFIDSLLNEENPSKAYRCSSKEAFEKGLCLSCRKNRCNNLGYEINKV
RAKASSKMYLKTRSQMPYKVFHYQVKIHFSGTESETHTNQAFEISLYGTVAESENIPFTLPEVSTNKTYSF
LIYTEVDIGELLMLKLKWKSDSYFSWSDWWSSPGFAIQKIRVKAGETQKKVIFCSREKVSHLQKGKAPA
VFVKCHDKSLNKKSGGGGGSGGGGSGGGGSGGGGSQTQQEEEEEDEDHGPDDYDEEDEDEVEEEE
TNRLPGGRSRVLLRCYTCKSLPRDERCNLTQNCSHGQTCTTLIAHGNTESGLLTTHSTWCTDSCQPITK
TVEGTQVTMTCCQSSLCNVPPWQSSRVQDPTGGGGGSGGGGSGGGGSDIQMTQSPSSVSASVGDR
VTITCQSSPSVWSNFLSWYQQKPGKAPKLLIYEASKLTSGVPSRFSGSGSGTDFTLTISSLQPEDFATYY
CGGGYSSISDTTFGGGTKVEIKGGGGSGGGGSGGGGSGGGGSEVQLLESGGGLVQPGGSLRLSCAV
SGIDLSNYAINWVRQAPGKGLEWIGIIWASGTTFYATWAKGRFTISRDNSKNTVYLQMNSLRAEDTAV
YYCARTVPGYSTAPYFDLWGQGTLVTVSS
(CA645Fab_HC(cotransf.CA645FabLC)-(G4S)3-hLPL(28-475)-(G4S)4-
hGPIHBP1(21-151)-FHA)
SEQ ID NO: 38
METDTLLLWVLLLWVPGSTGEVQLLESGGGLVQPGGSLRLSCAVSGIDLSNYAINWVRQAPGKGLEWI
GIIWASGTTFYATWAKGRFTISRDNSKNTVYLQMNSLRAEDTAVYYCARTVPGYSTAPYFDLWGQGTLV
TVSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCGGGGSADQRRDFIDIESKFALRTPEDTAEDT
CHLIPGVAESVATCHFNHSSKTFMVIHGWTVTGMYESWVPKLVAALYKREPDSNVIVVDWLSRAQEHY
PVSAGYTKLVGQDVARFINWMEEEFNYPLDNVHLLGYSLGAHAAGIAGSLTNKKVNRITGLDPAGPNFE
YAEAPSRLSPDDADFVDVLHTFTRGSPGRSIGIQKPVGHVDIYPNGGTFQPGCNIGEAIRVIAERGLGDV
DQLVKCSHERSIHLFIDSLLNEENPSKAYRCSSKEAFEKGLCLSCRKNRCNNLGYEINKVRAKRSSKMYL
KTRSQMPYKVFHYQVKIHFSGTESETHTNQAFEISLYGTVAESENIPFTLPEVSTNKTYSFLIYTEVDIGEL
LMLKLKWKSDSYFSWSDWWSSPGFAIQKIRVKAGETQKKVIFCSREKVSHLQKGKAPAVFVKCHDKSL
NKKSGGGGGSGGGGSGGGGSGGGGSQTQQEEEEEDEDHGPDDYDEEDEDEVEEEETNRLPGGRSR
VLLRCYTCKSLPRDERCNLTQNCSHGQTCTTLIAHGNTESGLLTTHSTWCTDSCQPITKTVEGTQVTMT
CCQSSLCNVPPWQSSRVQDPTGDYKDDDDKHHHHHHGGGLNDIFEAQKIEWHE
(NOV2704Fab_HC(cotransf. NOV2704Fab_LC)-(G4S)3-hLPL(28-475)-
(G4S)4-hGPIHBP1(21-151)-FHA)
SEQ ID NO: 39
METDTLLLWVLLLWVPGSTGEVQLLESGGGLVQPGGSLRLSCAASGFTFSDYAMSWVRQAPGKGLEW
VSAISYSGSYTYYADSVKGRFTISRDNSKNTLYLQMNSLRAEDTAVYYCARGRYGMDYWGQGTLVTVS
SASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCGGGGSGGGGSGGGGSADQRRDFIDIESKFALRT
PEDTAEDTCHLIPGVAESVATCHFNHSSKTFMVIHGWTVTGMYESWVPKLVAALYKREPDSNVIVVDW
LSRAQEHYPVSAGYTKLVGQDVARFINWMEEEFNYPLDNVHLLGYSLGAHAAGIAGSLTNKKVNRITGL
DPAGPNFEYAEAPSRLSPDDADFVDVLHTFTRGSPGRSIGIQKPVGHVDIYPNGGTFQPGCNIGEAIRVI
AERGLGDVDQLVKCSHERSIHLFIDSLLNEENPSKAYRCSSKEAFEKGLCLSCRKNRCNNLGYEINKVRA
KRSSKMYLKTRSQMPYKVFHYQVKIHFSGTESETHTNQAFEISLYGTVAESENIPFTLPEVSTNKTYSFLI
YTEVDIGELLMLKLKWKSDSYFSWSDWWSSPGFAIQKIRVKAGETQKKVIFCSREKVSHLQKGKAPAV
FVKCHDKSLNKKSGGGGGSGGGGSGGGGSGGGGSQTQQEEEEEDEDHGPDDYDEEDEDEVEEEET
NRLPGGRSRVLLRCYTCKSLPRDERCNLTQNCSHGQTCTTLIAHGNTESGLLTTHSTWCTDSCQPITKT
VEGTQVTMTCCQSSLCNVPPWQSSRVQDPTGDYKDDDDKHHHHHHGGGLNDIFEAQKIEWHE
(hLPL(28-475)-(GGGGS)₄-hGPIHBP1(21-151))
SEQ ID NO: 40
ADQRRDFIDIESKFALRTPEDTAEDTCHLIPGVAESVATCHFNHSSKTFMVIHGWTVTGMYESWVPKLV
AALYKREPDSNVIVVDWLSRAQEHYPVSAGYTKLVGQDVARFINWMEEEFNYPLDNVHLLGYSLGAHA
AGIAGSLTNKKVNRITGLDPAGPNFEYAEAPSRLSPDDADFVDVLHTFTRGSPGRSIGIQKPVGHVDIYP
NGGTFQPGCNIGEAIRVIAERGLGDVDQLVKCSHERSIHLFIDSLLNEENPSKAYRCSSKEAFEKGLCLS
CRKNRCNNLGYEINKVRAKRSSKMYLKTRSQMPYKVFHYQVKIHFSGTESETHTNQAFEISLYGTVAES
ENIPFTLPEVSTNKTYSFLIYTEVDIGELLMLKLKWKSDSYFSWSDWWSSPGFAIQKIRVKAGETQKKVI
FCSREKVSHLQKGKAPAVFVKCHDKSLNKKSGGGGGSGGGGSGGGGSGGGGSQTQQEEEEEDEDH
GPDDYDEEDEDEVEEEETNRLPGGRSRVLLRCYTCKSLPRDERCNLTQNCSHGQTCTTLIAHGNTESGL
LTTHSTWCTDSCQPITKTVEGTQVTMTCCQSSLCNVPPWQSSRVQDPTG
(His6-hLPL(28-475))
SEQ ID NO: 41
HHHHHHADQRRDFIDIESKFALRTPEDTAEDTCHLIPGVAESVATCHFNHSSKTFMVIHGWTVTGMYES
WVPKLVAALYKREPDSNVIVVDWLSRAQEHYPVSAGYTKLVGQDVARFINWMEEEFNYPLDNVHLLGY
SLGAHAAGIAGSLTNKKVNRITGLDPAGPNFEYAEAPSRLSPDDADFVDVLHTFTRGSPGRSIGIQKPVG
HVDIYPNGGTFQPGCNIGEAIRVIAERGLGDVDQLVKCSHERSIHLFIDSLLNEENPSKAYRCSSKEAFE
KGLCLSCRKNRCNNLGYEINKVRAKRSSKMYLKTRSQMPYKVFHYQVKIHFSGTESETHTNQAFEISLY
GTVAESENIPFTLPEVSTNKTYSFLIYTEVDIGELLMLKLKWKSDSYFSWSDWWSSPGFAIQKIRVKAGE
TQKKVIFCSREKVSHLQKGKAPAVFVKCHDKSLNKKSG
(Human LPL(28-475)-FHA)
SEQ ID NO: 42
ADQRRDFIDIESKFALRTPEDTAEDTCHLIPGVAESVATCHFNHSSKTFMVIHGWTVTGMYESWVPKLV
AALYKREPDSNVIVVDWLSRAQEHYPVSAGYTKLVGQDVARFINWMEEEFNYPLDNVHLLGYSLGAHA
AGIAGSLTNKKVNRITGLDPAGPNFEYAEAPSRLSPDDADFVDVLHTFTRGSPGRSIGIQKPVGHVDIYP
NGGTFQPGCNIGEAIRVIAERGLGDVDQLVKCSHERSIHLFIDSLLNEENPSKAYRCSSKEAFEKGLCLS
CRKNRCNNLGYEINKVRAKRSSKMYLKTRSQMPYKVFHYQVKIHFSGTESETHTNQAFEISLYGTVAES
ENIPFTLPEVSTNKTYSFLIYTEVDIGELLMLKLKWKSDSYFSWSDWWSSPGFAIQKIRVKAGETQKKVI
FCSREKVSHLQKGKAPAVFVKCHDKSLNKKSGDYKDDDDKHHHHHHGGGLNDIFEAQKIEWHE
(Human ANGPTL4(26-406)-FLAG-6HIS-Avi)
SEQ ID NO: 43
GPVQSKSPRFASWDEMNVLAHGLLQLGQGLREHAERTRSQLSALERRLSACGSACQGTEGSTDLPLAP
ESRVDPEVLHSLQTQLKAQNSRIQQLFHKVAQQQRHLEKQHLRIQHLQSQFGLLDHKHLDHEVAKPAR
RKRLPEMAQPVDPAHNVSRLHRLPRDCQELFQVGERQSGLFEIQPQGSPPFLVNCKMTSDGGWTVIQR
RHDGSVDFNRPWEAYKAGFGDPHGEFWLGLEKVHSITGDRNSRLAVQLRDWDGNAELLQFSVHLGGE
DTAYSLQLTAPVAGQLGATTVPPSGLSVPFSTWDQDHDLRRDKNCAKSLSGGWWFGTCSHSNLNGQY
FRSIPQQRQKLKKGIFWKTWRGRYYPLQATTMLIQPMAAEAASDYKDDDDKHHHHHHGGGLNDIFEA
QKIEWHE
(Nucleotide sequence: Human LPL(28-475)-(G4S)4-human GPIHBP1(22-
151)-FLAG-6HIS-Avi)
SEQ ID NO: 44
ATGGAAACCGACACCCTGCTGCTGTGGGTGCTGCTGCTGTGGGTGCCAGGATCTACAGGCGCCGAC
CAGCGGAGAGACTTCATCGACATCGAGAGCAAGTTCGCCCTGCGGACCCCTGAGGATACCGCCGAG
GATACCTGCCACCTGATTCCAGGCGTGGCCGAGAGCGTGGCCACCTGTCACTTCAACCACAGCTCC
AAGACCTTCATGGTCATCCACGGCTGGACCGTGACCGGGATGTACGAGAGCTGGGTGCCAAAACTG
GTGGCCGCCCTGTACAAGCGCGAGCCCGACAGCAATGTGATCGTGGTGGACTGGCTGAGCAGAGC
CCAGGAACACTACCCTGTGTCCGCCGGCTACACCAAGCTCGTGGGACAGGACGTGGCCCGGTTCAT
CAACTGGATGGAAGAAGAGTTCAACTACCCCCTGGACAATGTGCATCTGCTGGGCTACAGCCTGGG
AGCCCATGCCGCTGGAATTGCCGGCAGCCTGACCAACAAGAAAGTGAACCGGATCACCGGCCTGGA
CCCTGCCGGCCCTAATTTCGAGTATGCCGAGGCCCCCAGCAGACTGAGCCCCGACGATGCCGATTT
CGTGGACGTGCTGCACACCTTCACCAGAGGAAGCCCCGGCAGATCCATCGGCATCCAGAAACCTGT
GGGCCACGTGGACATCTACCCCAACGGCGGCACATTTCAGCCCGGCTGCAATATCGGCGAGGCCAT
CAGAGTGATCGCCGAGAGGGGCCTGGGAGATGTGGATCAGCTCGTGAAGTGCAGCCACGAGCGGA
GCATCCACCTGTTCATCGACTCCCTGCTGAACGAGGAAAACCCCAGCAAGGCCTACCGGTGCAGCA
GCAAAGAGGCCTTCGAGAAGGGCCTGTGCCTGAGCTGCCGGAAGAACCGGTGCAACAACCTGGGC
TACGAGATCAACAAAGTGCGGGCCAAGCGGAGCAGCAAGATGTACCTGAAAACCCGGTCCCAGATG
CCCTACAAGGTGTTCCATTATCAAGTGAAGATCCACTTCAGCGGCACCGAGAGCGAGACACACACCA
ACCAGGCCTTTGAGATCAGCCTGTACGGCACCGTGGCCGAATCCGAGAACATCCCCTTCACCCTGCC
CGAGGTGTCCACAAACAAGACCTACAGCTTCCTGATCTACACCGAGGTGGACATCGGCGAGCTGCT
GATGCTGAAGCTGAAATGGAAGTCCGACAGCTACTTCTCTTGGAGCGACTGGTGGTCCAGCCCCGG
CTTCGCCATTCAGAAAATTAGAGTGAAGGCCGGCGAAACCCAGAAAAAAGTGATCTTCTGCTCCCGC
GAGAAGGTGTCCCATCTGCAGAAGGGCAAAGCCCCTGCCGTGTTTGTGAAGTGCCACGACAAGAGC
CTGAACAAGAAGTCTGGCGGCGGAGGCGGATCTGGGGGAGGCGGAAGTGGCGGGGGAGGATCAG
GGGGCGGAGGATCTCAGACCCAGCAGGAAGAGGAAGAAGAGGACGAGGACCACGGCCCCGATGA
CTACGACGAAGAGGATGAGGATGAGGTGGAAGAAGAAGAGACAAACCGGCTGCCTGGCGGCAGGT
CCAGAGTGCTGCTGAGATGCTACACATGCAAGTCCCTGCCCCGGGACGAGCGGTGCAACCTGACAC
AGAATTGCTCCCACGGCCAGACCTGCACCACCCTGATCGCCCACGGCAATACCGAGTCTGGCCTGC
TGACCACCCACTCCACCTGGTGCACCGATAGCTGCCAGCCCATCACCAAGACCGTGGAAGGCACCC
AAGTGACCATGACCTGCTGCCAGTCCAGCCTGTGCAACGTGCCACCTTGGCAGAGCAGCAGAGTGC
AGGACCCCACCGGCGACTACAAGGACGACGACGACAAGCACCACCACCATCACCACGGCGGAGGA
CTGAACGACATCTTCGAGGCCCAGAAAATCGAGTGGCACGAATGA
(Mature human LPL R294A)
SEQ ID NO: 45
ADQRRDFIDIESKFALRTPEDTAEDTCHLIPGVAESVATCHFNHSSKTFMVIHGWTVTGMYESWVPKLV
AALYKREPDSNVIVVDWLSRAQEHYPVSAGYTKLVGQDVARFINWMEEEFNYPLDNVHLLGYSLGAHA
AGIAGSLTNKKVNRITGLDPAGPNFEYAEAPSRLSPDDADFVDVLHTFTRGSPGRSIGIQKPVGHVDIYP
NGGTFQPGCNIGEAIRVIAERGLGDVDQLVKCSHERSIHLFIDSLLNEENPSKAYRCSSKEAFEKGLCLS
CRKNRCNNLGYEINKVAAKRSSKMYLKTRSQMPYKVFHYQVKIHFSGTESETHTNQAFEISLYGTVAES
ENIPFTLPEVSTNKTYSFLIYTEVDIGELLMLKLKWKSDSYFSWSDWWSSPGFAIQKIRVKAGETQKKVI
FCSREKVSHLQKGKAPAVFVKCHDKSLNKKSG
(AHF-hLPL(28-475)R294A-(GGGGS)₄-hGPIHBP1(21-151))
SEQ ID NO: 46
METDTLLLWVLLLWVPGSTGGLNDIFEAQKIEWHEGGHHHHHHDYKDDDDKADQRRDFIDIESKFALR
TPEDTAEDTCHLIPGVAESVATCHFNHSSKTFMVIHGWTVTGMYESWVPKLVAALYKREPDSNVIVVD
WLSRAQEHYPVSAGYTKLVGQDVARFINWMEEEFNYPLDNVHLLGYSLGAHAAGIAGSLTNKKVNRIT
GLDPAGPNFEYAEAPSRLSPDDADFVDVLHTFTRGSPGRSIGIQKPVGHVDIYPNGGTFQPGCNIGEAIR
VIAERGLGDVDQLVKCSHERSIHLFIDSLLNEENPSKAYRCSSKEAFEKGLCLSCRKNRCNNLGYEINKV
AAKRSSKMYLKTRSQMPYKVFHYQVKIHFSGTESETHTNQAFEISLYGTVAESENIPFTLPEVSTNKTYSF
LIYTEVDIGELLMLKLKWKSDSYFSWSDWWSSPGFAIQKIRVKAGETQKKVIFCSREKVSHLQKGKAPA
VFVKCHDKSLNKKSGGGGGSGGGGSGGGGSGGGGSQTQQEEEEEDEDHGPDDYDEEDEDEVEEEE
TNRLPGGRSRVLLRCYTCKSLPRDERCNLTQNCSHGQTCTTLIAHGNTESGLLTTHSTWCTDSCQPITK
TVEGTQVTMTCCQSSLCNVPPWQSSRVQDPTG
(AHF-hLPL(28-475)R294A-(GGGGS)4-hGPIHBP1(21-151)(no signal
sequence))
SEQ ID NO: 47
GLNDIFEAQKIEWHEGGHHHHHHDYKDDDDKADQRRDFIDIESKFALRTPEDTAEDTCHLIPGVAESV
ATCHFNHSSKTFMVIHGWTVTGMYESWVPKLVAALYKREPDSNVIVVDWLSRAQEHYPVSAGYTKLVG
QDVARFINWMEEEFNYPLDNVHLLGYSLGAHAAGIAGSLTNKKVNRITGLDPAGPNFEYAEAPSRLSPD
DADFVDVLHTFTRGSPGRSIGIQKPVGHVDIYPNGGTFQPGCNIGEAIRVIAERGLGDVDQLVKCSHER
SIHLFIDSLLNEENPSKAYRCSSKEAFEKGLCLSCRKNRCNNLGYEINKVAAKRSSKMYLKTRSQMPYKV
FHYQVKIHFSGTESETHTNQAFEISLYGTVAESENIPFTLPEVSTNKTYSFLIYTEVDIGELLMLKLKWKSD
SYFSWSDWWSSPGFAIQKIRVKAGETQKKVIFCSREKVSHLQKGKAPAVFVKCHDKSLNKKSGGGGG
SGGGGSGGGGSGGGGSQTQQEEEEEDEDHGPDDYDEEDEDEVEEEETNRLPGGRSRVLLRCYTCKSL
PRDERCNLTQNCSHGQTCTTLIAHGNTESGLLTTHSTWCTDSCQPITKTVEGTQVTMTCCQSSLCNVPP
WQSSRVQDPTG
(hLPL(28-475)R294A-(GGGGS)₄-hGPIHBP1(21-151))
SEQ ID NO: 48
ADQRRDFIDIESKFALRTPEDTAEDTCHLIPGVAESVATCHFNHSSKTFMVIHGWTVTGMYESWVPKLV
AALYKREPDSNVIVVDWLSRAQEHYPVSAGYTKLVGQDVARFINWMEEEFNYPLDNVHLLGYSLGAHA
AGIAGSLTNKKVNRITGLDPAGPNFEYAEAPSRLSPDDADFVDVLHTFTRGSPGRSIGIQKPVGHVDIYP
NGGTFQPGCNIGEAIRVIAERGLGDVDQLVKCSHERSIHLFIDSLLNEENPSKAYRCSSKEAFEKGLCLS
CRKNRCNNLGYEINKVAAKRSSKMYLKTRSQMPYKVFHYQVKIHFSGTESETHTNQAFEISLYGTVAES
ENIPFTLPEVSTNKTYSFLIYTEVDIGELLMLKLKWKSDSYFSWSDWWSSPGFAIQKIRVKAGETQKKVI
FCSREKVSHLQKGKAPAVFVKCHDKSLNKKSGGGGGSGGGGSGGGGSGGGGSQTQQEEEEEDEDH
GPDDYDEEDEDEVEEEETNRLPGGRSRVLLRCYTCKSLPRDERCNLTQNCSHGQTCTTLIAHGNTESGL
LTTHSTWCTDSCQPITKTVEGTQVTMTCCQSSLCNVPPWQSSRVQDPTG
(Human soluble GPIHBP1(21-151)-FLAG-HIS₆-Avi)
SEQ ID NO: 49
QTQQEEEEEDEDHGPDDYDEEDEDEVEEEETNRLPGGRSRVLLRCYTCKSLPRDERCNLTQNCSHGQT
CTTLIAHGNTESGLLTTHSTWCTDSCQPITKTVEGTQVTMTCCQSSLCNVPPWQSSRVQDPTGDYKDD
DDKHHHHHHGGGLNDIFEAQKIEWHE
(Human ANGPTL3(17-460)-FLAG-HIS₆-Avi)
SEQ ID NO: 50
SRIDQDNSSFDSLSPEPKSRFAMLDDVKILANGLLQLGHGLKDFVHKTKGQINDIFQKLNIFDQSFYDLS
LQTSEIKEEEKELRRTTYKLQVKNEEVKNMSLELNSKLESLLEEKILLQQKVKYLEEQLTNLIQNQPETPEH
PEVTSLKTFVEKQDNSIKDLLQTVEDQYKQLNQQHSQIKEIENQLRRTSIQEPTEISLSSKPRAPRTTPFL
QLNEIRNVKHDGIPAECTTIYNRGEHTSGMYAIRPSNSQVFHVYCDVISGSPWTLIQHRIDGSQNFNET
WENYKYGFGRLDGEFWLGLEKIYSIVKQSNYVLRIELEDWKDNKHYIEYSFYLGNHETNYTLHLVAITGN
VPNAIPENKDLVFSTWDHKAKGHFNCPEGYSGGWWWHDECGENNLNGKYNKPRAKSKPERRRGLSW
KSQNGRLYSIKSTKMLIHPTDSESFEDYKDDDDKHHHHHHGGGLNDIFEAQKIEWHE
(LPL(28-475)-(G4S)4-hGPIHBP1(21-151)-FLAG-HIS₆-Avi)
SEQ ID NO: 51
METDTLLLWVLLLWVPGSTGADQRRDFIDIESKFALRTPEDTAEDTCHLIPGVAESVATCHFNHSSKTFM
VIHGWTVTGMYESWVPKLVAALYKREPDSNVIVVDWLSRAQEHYPVSAGYTKLVGQDVARFINWMEEE
FNYPLDNVHLLGYSLGAHAAGIAGSLTNKKVNRITGLDPAGPNFEYAEAPSRLSPDDADFVDVLHTFTRG
SPGRSIGIQKPVGHVDIYPNGGTFQPGCNIGEAIRVIAERGLGDVDQLVKCSHERSIHLFIDSLLNEENP
SKAYRCSSKEAFEKGLCLSCRKNRCNNLGYEINKVRAKRSSKMYLKTRSQMPYKVFHYQVKIHFSGTES
ETHTNQAFEISLYGTVAESENIPFTLPEVSTNKTYSFLIYTEVDIGELLMLKLKWKSDSYFSWSDWWSSP
GFAIQKIRVKAGETQKKVIFCSREKVSHLQKGKAPAVFVKCHDKSLNKKSGGGGGSGGGGSGGGGSG
GGGSQTQQEEEEEDEDHGPDDYDEEDEDEVEEEETNRLPGGRSRVLLRCYTCKSLPRDERCNLTQNCS
HGQTCTTLIAHGNTESGLLTTHSTWCTDSCQPITKTVEGTQVTMTCCQSSLCNVPPWQSSRVQDPTGD
YKDDDDKHHHHHHGGGLNDIFEAQKIEWHE
(signal peptide)
SEQ ID NO: 52
METDTLLLWVLLLWVPGSTG
(LPL(28-475)-(G4S)4-hGPIHBP1(21-151)-FLAG-HIS6-Avi)
SEQ ID NO: 53
ADQRRDFIDIESKFALRTPEDTAEDTCHLIPGVAESVATCHFNHSSKTFMVIHGWTVTGMYESWVPKLV
AALYKREPDSNVIVVDWLSRAQEHYPVSAGYTKLVGQDVARFINWMEEEFNYPLDNVHLLGYSLGAHA
AGIAGSLTNKKVNRITGLDPAGPNFEYAEAPSRLSPDDADFVDVLHTFTRGSPGRSIGIQKPVGHVDIYP
NGGTFQPGCNIGEAIRVIAERGLGDVDQLVKCSHERSIHLFIDSLLNEENPSKAYRCSSKEAFEKGLCLS
CRKNRCNNLGYEINKVRAKRSSKMYLKTRSQMPYKVFHYQVKIHFSGTESETHTNQAFEISLYGTVAES
ENIPFTLPEVSTNKTYSFLIYTEVDIGELLMLKLKWKSDSYFSWSDWWSSPGFAIQKIRVKAGETQKKVI
FCSREKVSHLQKGKAPAVFVKCHDKSLNKKSGGGGGSGGGGSGGGGSGGGGSQTQQEEEEEDEDH
GPDDYDEEDEDEVEEEETNRLPGGRSRVLLRCYTCKSLPRDERCNLTQNCSHGQTCTTLIAHGNTESGL
LTTHSTWCTDSCQPITKTVEGTQVTMTCCQSSLCNVPPWQSSRVQDPTGDYKDDDDKHHHHHHGGG
LNDIFEAQKIEWHE
(LPL(28-475)-(G4S)4-hGPIHBP1(21-151))
SEQ ID NO: 54
ADQRRDFIDIESKFALRTPEDTAEDTCHLIPGVAESVATCHFNHSSKTFMVIHGWTVTGMYESWVPKLV
AALYKREPDSNVIVVDWLSRAQEHYPVSAGYTKLVGQDVARFINWMEEEFNYPLDNVHLLGYSLGAHA
AGIAGSLTNKKVNRITGLDPAGPNFEYAEAPSRLSPDDADFVDVLHTFTRGSPGRSIGIQKPVGHVDIYP
NGGTFQPGCNIGEAIRVIAERGLGDVDQLVKCSHERSIHLFIDSLLNEENPSKAYRCSSKEAFEKGLCLS
CRKNRCNNLGYEINKVRAKRSSKMYLKTRSQMPYKVFHYQVKIHFSGTESETHTNQAFEISLYGTVAES
ENIPFTLPEVSTNKTYSFLIYTEVDIGELLMLKLKWKSDSYFSWSDWWSSPGFAIQKIRVKAGETQKKVI
FCSREKVSHLQKGKAPAVFVKCHDKSLNKKSGGGGGSGGGGSGGGGSGGGGSQTQQEEEEEDEDH
GPDDYDEEDEDEVEEEETNRLPGGRSRVLLRCYTCKSLPRDERCNLTQNCSHGQTCTTLIAHGNTESGL
LTTHSTWCTDSCQPITKTVEGTQVTMTCCQSSLCNVPPWQSSRVQDPTG
(hLPL(28-475)-(G4S)4-hGPIHBP1(21-151))
SEQ ID NO: 55
ADQRRDFIDIESKFALRTPEDTAEDTCHLIPGVAESVATCHFNHSSKTFMVIHGWTVTGMYESWVPKLV
AALYKREPDSNVIVVDWLSRAQEHYPVSAGYTKLVGQDVARFINWMEEEFNYPLDNVHLLGYSLGAHA
AGIAGSLTNKKVNRITGLDPAGPNFEYAEAPSRLSPDDADFVDVLHTFTRGSPGRSIGIQKPVGHVDIYP
NGGTFQPGCNIGEAIRVIAERGLGDVDQLVKCSHERSIHLFIDSLLNEENPSKAYRCSSKEAFEKGLCLS
CRKNRCNNLGYEINKVRAKRSSKMYLKTRSQMPYKVFHYQVKIHFSGTESETHTNQAFEISLYGTVAES
ENIPFTLPEVSTNKTYSFLIYTEVDIGELLMLKLKWKSDSYFSWSDWWSSPGFAIQKIRVKAGETQKKVI
FCSREKVSHLQKGKAPAVFVKCHDKSLNKKSGGGGGSGGGGSGGGGSGGGGSQTQQEEEEEDEDH
GPDDYDEEDEDEVEEEETNRLPGGRSRVLLRCYTCKSLPRDERCNLTQNCSHGQTCTTLIAHGNTESGL
LTTHSTWCTDSCQPITKTVEGTQVTMTCCQSSLCNVPPWQSSRVQDPTG

1. A fusion polypeptide comprising:
(i) a lipoprotein lipase (LPL) polypeptide, or a functional variant thereof; and
(ii) a glycosylphosphatidylinositol-anchored High Density Lipoprotein-binding protein 1 (GPIHBP1) polypeptide, or a functional variant thereof.

2. The fusion polypeptide according to claim 1, having one formula (I) or (II)
A-B₍ₙ₎-C-D₍ₘ₎-E (I)
A-D₍ₘ₎-C-B₍ₙ₎-E (II)
wherein
A=an optional N-terminal sequence
B=LPL polypeptide or functional variant thereof
C=an optional linker sequence
D=GPIHBP1 polypeptide or functional variant thereof
E=an optional C-terminal sequence, wherein
n=an integer from 1 to 3, and
m=an integer from 1 to 3.

3. The fusion polypeptide according to claim 1 or claim 2, wherein n=1 and/or m=1.

4. The fusion polypeptide according to any one of the previous claims, wherein the functional variant of the LPL polypeptide comprises an amino acid sequence having at least about 80%, at least about 85%, at least about 90%, at least about 95%, at least about 97%, at least about 98%, or at least about 99% sequence identity to SEQ ID NO: 1 or SEQ ID NO:2.

5. The fusion polypeptide according to any one of the previous claims, wherein the functional variant of the LPL polypeptide comprises the amino acid sequence of
(i) SEQ ID NO: 2 having one or more (e.g., 1, 2, 3, 4, 5, 6, 7, 8, 9, 10 or more) point mutations that add to, delete, or substitute any of the amino acids of SEQ ID NO: 2, or
(ii) SEQ ID NO: 1 having one or more (e.g., 1, 2, 3, 4, 5, 6, 7, 8, 9, 10 or more) point mutations that add to, delete, or substitute any of amino acids 157-189 of SEQ ID NO: 1.

6. The fusion polypeptide according to any one of the previous claims 1 to 3, wherein the functional variant of the LPL polypeptide is a truncated version of
(i) an amino acid sequence having at least about 80%, at least about 85%, at least about 90%, at least about 95%, at least about 97%, at least about 98%, or at least about 99% sequence identity to SEQ ID NO: 1 or SEQ ID NO:2;
(ii) SEQ ID NO: 2 having one or more (e.g., 1, 2, 3, 4, 5, 6, 7, 8, 9, 10 or more) point mutations that add to, delete, or substitute any of the amino acids of SEQ ID NO: 2,
(iii) SEQ ID NO: 1 having one or more (e.g., 1, 2, 3, 4, 5, 6, 7, 8, 9, 10 or more) point mutations that add to, delete, or substitute any of amino acids 157-189 of SEQ ID NO: 1;
(iv) SEQ ID NO: 1; or
(v) SEQ ID NO: 2.

7. The fusion polypeptide according to any one of claims 1 to 3, wherein the LPL polypeptide comprises or consists of any one of SEQ ID NOs: 1, 2, 3, 4, or 45.

8. The fusion polypeptide according to any one of the previous claims, wherein the functional variant of the GPIHBP1 polypeptide comprises an amino acid sequence having at least about 80%, at least about 85%, at least about 90%, at least about 95%, at least about 97%, at least about 98% or at least about 99% sequence identity to the GPIHBP1 polypeptide of SEQ ID NO: 5, SEQ ID NO: 6 or SEQ ID NO: 7.

9. The fusion polypeptide according to any one of the previous claims, wherein the functional variant of the GPIHBP1 polypeptide comprises the amino acid sequence of SEQ ID NO: 7 having one or more (e.g., 1, 2, 3, 4, 5, 6, 7, 8, 9, 10 or more) point mutations that add to, delete, or substitute any of the amino acids of SEQ ID NO: 7.

10. The fusion polypeptide according to any one of the previous claims 1 to 7, wherein the functional variant of the GPIHBP1 polypeptide is a truncated version of (i) an amino acid sequence having at least about 80%, at least about 85%, at least about 90%, at least about 95%, at least about 97%, at least about 98% or at least about 99% sequence identity to the GPIHBP1 polypeptide of SEQ ID NO: 5; or
(ii) SEQ ID NO: 5.

11. The fusion polypeptide according to any one of claims 1 to 7, wherein the GPIHBP1 polypeptide comprises or consists of any one of SEQ ID NOs: 5, 6, 7, 8, 9 or 10.

12. The fusion polypeptide according to any one of the previous claims, wherein the LPL polypeptide and GPIHBP1 polypeptide are joined by a linker C.

13. The fusion polypeptide according to claim 12, wherein the linker comprises or consists of one or more of the amino acid sequences recited in any one of SEQ ID NOs: 11-27.

14. The fusion polypeptide according to claim 13, wherein the linker comprises or consists of one or more of the amino acid sequence recited in SEQ ID NO: 16 or SEQ ID NO: 17.

15. The fusion polypeptide according to any one of the previous claims, wherein the fusion polypeptide comprises an N-terminal sequence A.

16. The fusion polypeptide according to any one of the previous claims, wherein the fusion polypeptide comprises a C-terminal sequence E.

17. The fusion polypeptide according to claim 15 or claim 16, wherein the N-terminal or C-terminal sequence comprises one or more tags selected from the group consisting of a His-tag, a FLAG-tag, Arg-tag, T7-tag, Strep-tag, S-tag, an AviTag™ and an aptamer-tag.

18. The fusion polypeptide according to claim 17, wherein the N-terminal or C-terminal sequence comprises
(i) a His-tag and an AviTag™, or
(ii) a FLAG-tag, a His-tag and an AviTag™.

19. The fusion polypeptide according to claim 15 or claim 16, wherein the N-terminal or C-terminal sequence comprises or consists of the amino acid sequence recited in SEQ ID NO: 31 or SEQ ID NO: 32.

20. The fusion polypeptide according to any one of the previous claims, wherein the N-terminal or C-terminal sequence comprises a moiety to increase the half-life of the fusion polypeptide in vivo,
and wherein the N-terminal or C-terminal sequence comprises a PEG sequence, a PAS sequence or an antibody sequence, optionally selected from a Fab or ScFv molecule.

21. The fusion polypeptide according to claim 20, wherein the antibody is CA645.

22. The fusion polypeptide according to any of claims 1-3, comprising or consisting of the amino acid sequence of any of SEQ ID NOs: 33-40, 46-48, 51, 53, 54, or 55.

23. An isolated nucleic acid sequence encoding a fusion polypeptide according to any one of the previous claims 1 to 22.

24. A vector comprising a nucleic acid according to claim 23.

25. A host cell comprising a nucleic acid according to claim 23 or a vector according to claim 24.

26. A method for making an the fusion polypeptide according to any one of claims 1 to 22, comprising maintaining the host cell of claim 25 under conditions suitable for expression of a nucleic acid, whereby a nucleic acid is expressed and the fusion polypeptide is produced, and optionally isolating and/or purifying the fusion polypeptide.

27. A pharmaceutical composition comprising the fusion polypeptide according to any one of claims 1 to 22, the nucleic acid according to claim 23, the vector according to claim 24 or the host cell according to claim 25, and a pharmaceutically or physiologically acceptable diluent and/or carrier.

28. The fusion polypeptide according to any one of claims 1-22, the nucleic acid according to claim 23, the vector according to claim 24, the host cell according to claim 25 or the pharmaceutical composition according to claim 27 for use in therapy or for use as a medicament for the treatment of a disease or disorder.

29. The fusion polypeptide, nucleic acid, vector, host cell or pharmaceutical composition for use according to claim 28, wherein the disease or disorder is selected from chylomicronemia (including Familial chylomicronemia syndrome, polygenic late-onset chylomicronemia and early-onset chylomicronemia), hyperlipidemic pancreatitis, hypertriglyceridemia, abdominal pain, recurrent acute pancreatitis, eruptive cutaneous xanthomata, lipemia retinalis, hepatosplenomegaly, diabetes, obesity, cardiovascular disease, chronic kidney disease, non-alcoholic fatty liver disease, hypertriglyceridemic pancreatitis, hepatosteatosis, metabolic syndrome, ischemic heart disease and microvascular pathology.

30. A method of treating a patient suffering from a disease or disorder comprising administering a therapeutically effective amount of the fusion polypeptide according to any one of claims 1-22, the nucleic acid according to claim 23, the vector according to claim 24, the host cell according to claim 25 or the pharmaceutical composition according to claim 27 to said patient.

31. The method according to claim 30, wherein the disease or disorder is selected from chylomicronemia (including Familial chylomicronemia syndrome, polygenic late-onset chylomicronemia and early-onset chylomicronemia), hyperlipidemic pancreatitis, hypertriglyceridemia, abdominal pain, recurrent acute pancreatitis, eruptive cutaneous xanthomata, lipemia retinalis, hepatosplenomegaly, diabetes, obesity, cardiovascular disease, chronic kidney disease, non-alcoholic fatty liver disease, hypertriglyceridemic pancreatitis, hepatosteatosis, metabolic syndrome, ischemic heart disease and microvascular pathology.
